SEQUENCE LISTING (23 sequences)

SEQ ID NO: 1 - protein, 15 aa, Homo sapiens
QSLLGDYDHYYGLDA

SEQ ID NO: 2 - protein, 15 aa, Homo sapiens
QSLLGDYDHYYGLDV

SEQ ID NO: 3 - protein, 16 aa, Homo sapiens
DQSLLGDYDHYYGLDV

SEQ ID NO: 4 - protein, 15 aa, Homo sapiens
QSLLGDYDRYYGLDA

SEQ ID NO: 5 - protein, 15 aa, Homo sapiens
QSLLGDYDKYYGLDA

SEQ ID NO: 6 - protein, 16 aa, Homo sapiens
  VARIANT 1: note = when present, is D
  VARIANT 10: note = H, R or K
  VARIANT 16: note = A or V
XQSLLGDYDXYYGLDX

SEQ ID NO: 7 - protein, 261 aa, Homo sapiens
QVQLVESGGGVVQPGRSLRLSCAASGFPFRSFAMHWVRQALGKGLEWVAVISYDGSTKYYADSVKGRFTISRDTSKNTVYLKMNRLRTEDTAVYYCARQSLLGDYDHYYGLDAWGKGTTVTVSSGGGGSGGGGSGGGGSDIVLTQSPGTLSLSPGERATLSCRASQSVSSSYLAWYQQKPGQAPRLLIYGASTRATGMPDRFSGSGSGTDFTLTISRLEPEDFAVYYCQQYGSSPQTPQITFGGGTKVEIKHHHHHHHHHH

SEQ ID NO: 8 - protein, 261 aa, Homo sapiens
QVQLVESGGGVVQPGRSLRLSCAASGFPFRSFAMHWVRQALGKGLEWVAVISYDGSTKYYADSVKGRFTISRDTSKNTVYLKMNRLRTEDTAVYYCARQSLLGDYDHYYGLDVWGKGTTVTVSSGGGGSGGGGSGGGGSDIVLTQSPGTLSLSPGERATLSCRASQSVSSSYLAWYQQKPGQAPRLLIYGASTRATGMPDRFSGSGSGTDFTLTISRLEPEDFAVYYCQQYGSSPQTPQITFGGGTKVEIKHHHHHHHHHH

SEQ ID NO: 9 - protein, 262 aa, Homo sapiens
QVQLVESGGGVVQPGRSLRLSCAASGFPFRSFAMHWVRQALGKGLEWVAVISYDGSTKYYADSVKGRFTISRDTSKNTVYLKMNRLRTEDTAVYYCARDQSLLGDYDHYYGLDVWGKGTTVTVSSGGGGSGGGGSGGGGSDIVLTQSPGTLSLSPGERATLSCRASQSVSSSYLAWYQQKPGQAPRLLIYGASTRATGMPDRFSGSGSGTDFTLTISRLEPEDFAVYYCQQYGSSPQTPQITFGGGTKVEIKHHHHHHHHHH

SEQ ID NO: 10 - protein, 261 aa, Homo sapiens
QVQLVESGGGVVQPGRSLRLSCAASGFPFRSFAMHWVRQALGKGLEWVAVISYDGSTKYYADSVKGRFTISRDTSKNTVYLKMNRLRTEDTAVYYCARQSLLGDYDRYYGLDAWGKGTTVTVSSGGGGSGGGGSGGGGSDIVLTQSPGTLSLSPGERATLSCRASQSVSSSYLAWYQQKPGQAPRLLIYGASTRATGMPDRFSGSGSGTDFTLTISRLEPEDFAVYYCQQYGSSPQTPQITFGGGTKVEIKHHHHHHHHHH

SEQ ID NO: 11 - protein, 261 aa, Homo sapiens
QVQLVESGGGVVQPGRSLRLSCAASGFPFRSFAMHWVRQALGKGLEWVAVISYDGSTKYYADSVKGRFTISRDTSKNTVYLKMNRLRTEDTAVYYCARQSLLGDYDKYYGLDAWGKGTTVTVSSGGGGSGGGGSGGGGSDIVLTQSPGTLSLSPGERATLSCRASQSVSSSYLAWYQQKPGQAPRLLIYGASTRATGMPDRFSGSGSGTDFTLTISRLEPEDFAVYYCQQYGSSPQTPQITFGGGTKVEIKHHHHHHHHHH

SEQ ID NO: 12 - protein, 5 aa, Homo sapiens
SFAMH

SEQ ID NO: 13 - protein, 17 aa, Homo sapiens
VISYDGSTKYYADSVKG

SEQ ID NO: 14 - protein, 12 aa, Homo sapiens
RASQSVSSSYLA

SEQ ID NO: 15 - protein, 7 aa, Homo sapiens
GASTRAT

SEQ ID NO: 16 - protein, 13 aa, Homo sapiens
QQYGSSPQTPQIT

SEQ ID NO: 17 - protein, 104 aa, Homo sapiens
EVQLLESGGGLVQPGGSLRLSCAASGFTFSNYTMAWVRQAPGKGLEWVSSISSSGGHTPYADSVKGRFTISRDNSKNTLYLQMNSLRAEDTAVYYCARGKRNRN

SEQ ID NO: 18 - protein, 104 aa, Homo sapiens
QDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPT

SEQ ID NO: 19 - protein, 104 aa, Homo sapiens
EVQLLESGGGLVQPGGSLRLSCAASGFTFSEYIMSWVRQAPGKGLEWVSYISPSGGTTKYADSVKGRFTISRDNSKNTLYLQMNSLRAEDTAVYYCARGRRTKH

SEQ ID NO: 20 - protein, 104 aa, Homo sapiens
QDIQMTQSPLSSLPVTLGQSASISCRSSQSLVHSDGNTYLNWFQQRPGQSPRRLIYKVSNRDSGVPDRFSGSGSGTDFTLKISRVEAEDVGVYYCMQGTHWPYT

SEQ ID NO: 21 - protein, 259 aa, Homo sapiens
AMAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSLRMNGWRAHQWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAHHHHHHHHHH

SEQ ID NO: 22 - protein, 253 aa, Homo sapiens
EVQLLESGGGLVQPGGSLRLSCAASGFTFSNYTMAWVRQAPGKGLEWVSSISSSGGHTPYADSVKGRFTISRDNSKNTLYLQMNSLRAEDTAVYYCARGKRNRNWGQGTLVTVSSGGGGSGGGGSGGGGSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGQGTKLEIKHHHHHHHHHH

SEQ ID NO: 23 - protein, 252 aa, Homo sapiens
EVQLLESGGGLVQPGGSLRLSCAASGFTFSEYIMSWVRQAPGKGLEWVSYISPSGGTTKYADSVKGRFTISRDNSKNTLYLQMNSLRAEDTAVYYCARGRRTKHWGQGTLVTVSSGGGGSGGGGSGGGGSDIQMTQSPDSLAVSLGERATINCRSSQSLVHSDGNTYLNWYQQKPGQPPKLLIYKVSNRDSGVPDRFSGSGSGTDFTLTISSLQAEDVAVYYCMQGTHWPYTFGQGTKLEIKHHHHHHHHHH

(19) United States
(12) Patent Application Publication
(10) Pub. No.: US 2026/0234243 A1
(43) Pub. Date: Aug. 13, 2026

(54) ANTIBODIES TO GLYCOSAMINOGLYCANS

(71) Applicant: ABIDEA INC., Toronto (CA)

(72) Inventor: Michael Dan, Toronto (CA)

(73) Assignee: ABIDEA INC., Toronto, ON (CA)

(21) Appl. No.: 19/303,760

(22) Filed: Aug. 19, 2025

Related U.S. Application Data
(60) Provisional application No. 63/685,052, filed on Aug. 20, 2024.

(51) Int. Cl. C07K 16/28; A61K 45/06; A61P 35/00

(52) U.S. Cl. CPC C07K 16/28; A61K 45/06; A61P 35/00; C07K 2317/565; C07K 2317/622; C07K 2317/92

(57) ABSTRACT

Polyspecific anti-glycosaminoglycan (GAG) antibodies and methods of use thereof for directly killing cancer cells. The anti-GAG antibodies can be used for purging cancer cells from autologous stem cell transplants (ASCT) or autologous bone marrow transplants (ABMT). The cancer cells may be multiple myeloma, Hodgkin's lymphoma, neuroblastoma, or any other cancer that expresses a membrane-tethered heparan sulfate proteoglycan (HSPG), such as syndecan or glypican.

### CROSS REFERENCE TO RELATED APPLICATIONS

This application claims the benefit of U.S. Provisional Patent Application No. 63/685,052, filed Aug. 20, 2024, and entitled “ANTIBODIES TO GLYCOSAMINOGLYCANS,” the entirety of which is incorporated by reference herein.

### STATEMENT REGARDING A SEQUENCE LISTING

The present application contains a sequence listing entitled G10058305P1US.xml created Aug. 13, 2025 and is 24,151 bytes in size. The sequence listing is submitted electronically along with the filing of the present application and is hereby incorporated by reference in its entirety.

### TECHNOLOGICAL FIELD

The present disclosure relates to antibodies for the treatment of cancer. More particularly, the present disclosure relates to human antibodies and fragments thereof that bind to more than one class of isolated sulfated glycosaminoglycan (GAG) and their methods of use for the treatment of cancer.

### BACKGROUND

Growth factors bind to cells through a complex process involving both canonical receptors and glycosaminoglycans (GAGs) on the cell surface. This dual interaction mechanism allows for precise control and modulation of growth factor signaling, as exemplified by basic fibroblast growth factor (FGF2; Fannon, et al, 2000).

Canonical receptors are specific proteins expressed on the cell surface that recognize and bind to growth factors. These receptors are often transmembrane proteins with extracellular domains that can directly interact with growth factors. The binding of a growth factor to its canonical receptor initiates a series of intracellular signaling events that transmit the growth factor's signal into the cell. This can lead to various cellular responses, such as cell proliferation, differentiation, migration, and survival.

GAGs may be found on essentially all cell surfaces and in the extracellular matrix as they play important functional and structural roles. GAG complexity and functional specificity is the result of several post-translational modifications, including the site and degree of sulfation, as well as acetylation; the type of linkages between saccharide units within the chain; and a variety of sequence variations, since GAGs are not simple repeating disaccharides. As a result, GAGs contain highly specific biochemical information which accounts for their functional and organ specificity. They are among the most information-rich molecules in nature, second only to nucleic acids

GAGs may be further subdivided into sulfated GAGs which include heparin (Hep), heparan sulfate (HS), chondroitin sulfate (CS), dermatan sulfate (DS), and keratan sulfate (KS), and unsulfated GAGs which include hyaluronic acid (HA). Some GAGs (HS, CS, DS, and KS) are associated with a protein core, in which case they form proteoglycans, while others (Hep and HA) exist as isolated molecules. Biochemically and structurally, GAGs may also be viewed as belonging to one of four different classes: Hep/HS, CS/DS, KS, or HA.

The increased incidence of thrombosis among cancer patients has been known for nearly 150 years and is referred to as Trousseau syndrome. Today, Low Molecular Weight Heparin (LMWH) is the drug of choice for antithrombosis prophylaxis and treatment in cancer patients (Ay, et al. 2017) and it may also have a beneficial effect on cancer survival via mechanisms unrelated to the prevention of venous thrombosis (Borsig, 2010; Franchini and Mannucci, 2015; Atallah, et al. 2020). Hep can affect tumor cell proliferation, adhesion, angiogenesis, migration, and invasion via multiple mechanisms including inhibition of heparanase, P-/L-selectin, angiogenesis, and interference with the CXCL12-CXCR4 axis (Ma, et al. 2020).

Most of the biological roles of HSPGs are mediated by their HS chains through binding various protein ligands including cytokines, chemokines, growth factors and receptors, morphogens, proteases, protease inhibitors, and extracellular (ECM) proteins. In basement membranes, HSPGs collaborate with other matrix components to define basement membrane structure and to provide a matrix for cell migration and invasion. HSPG dysregulation may hijack these pathways to confer cellular malignancy leading to tumor development and progression (Yang and Wang, 2023). Sterically interfering with and blocking HS side chains of HSPGs raises the prospect of mitigating HSPG dysregulation. One means of achieving this goal is through HS-specific antibodies and antibody fragments.

There is thus a need to develop antibodies and antibody fragments that can block the effects of aberrant growth factor signaling on cancer cells through HS-specific interactions.

### SUMMARY OF THE INVENTION

The present disclosure concerns antibodies that that bind to heparan sulfate side chains of membrane-tethered core proteins and block the effects of growth factor signaling on cancer cells.

According to a first aspect, the present invention is directed to an antibody that binds to isolated sulfated glycosaminoglycans (GAGs), wherein the antibody comprises a Variable Heavy Chain Complementarity Determining Region 3 (VHCDR3) according to the Kabat numbering system having the amino acid sequence of SEQ ID NO: 6.

According to a preferred embodiment, the antibody is a single-chain variable fragment (scFv).

According to an embodiment, the antibody is a human scFv antibody.

According to an embodiment, said antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, the antibody comprises a VHCDR3 according to the Kabat numbering system having the amino acid sequence of any one of SEQ ID NO: 1-5.

According to an embodiment, the antibody described herein comprises a VHCDR3 according to the Kabat numbering system having the amino acid sequence of SEQ ID NO: 3.

According to an embodiment, the antibody comprises a VHCDR3 according to the Kabat numbering system having the amino acid sequence of SEQ ID NO: 1, 2, 4 or 5.

According to an embodiment, the antibody and heparin having a degree of polymerization of 24 (dp24) is less than 250 nM.

According to an embodiment, the K_{d} of the antibody and chondroitin sulfate D having a degree of polymerization of 20 (dp20) is less than 300 nM.

According to a second aspect, the present invention is directed to an antibody or fragment thereof with a VH or a VL region having at least 70%, at least 80%, or at least 90% homology to the VH or VL amino acid sequence of any one of SEQ ID NO: 7-11 and 21-23, wherein the antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to a preferred embodiment, the GAGs comprise isolated heparin, high sulfation heparan sulfate, low/intermediate sulfation heparan sulfate, and chondroitin-2,6-sulfate with 20 degrees of polymerization (dp20).

According to an embodiment, the antibody binds to isolated heparin, high sulfation heparan sulfate, low/intermediate sulfation heparan sulfate, and chondroitin-2,6-sulfate with 20 degrees of polymerization (dp20).

According to an embodiment, the K_{d} of the antibody and a heparan sulfate octamer with 2.5 sulfates/disaccharide (HS024) under mildly acidic conditions is less than 1 μM.

According to an embodiment, the mildly acidic conditions correspond to a pH of between 6.0 and 6.8.

According to an embodiment, the antibody comprises a methionine in the penultimate position of the VHCDR1 according to the Kabat numbering system.

According to an embodiment, the antibody comprises the amino acid sequence VKG at the C-terminal end of the VHCDR2 according to the Kabat numbering system.

According to an embodiment, the antibody comprises the amino acid sequence YLA at the C-terminal end of the VLCDR1 according to the Kabat numbering system.

According to an embodiment, the antibody comprises a VHCDR1 according to the Kabat numbering system of SEQ ID NO: 12.

According to an embodiment, the antibody comprises a VHCDR2 according to the Kabat numbering system of SEQ ID NO: 13.

According to an embodiment, the antibody comprises a VLCDR1 according to the Kabat numbering system of SEQ ID NO: 14.

According to an embodiment, the antibody comprises a VLCDR2 according to the Kabat numbering system of SEQ ID NO: 15.

According to an embodiment, the antibody comprises a VLCDR3 according to the Kabat numbering system of SEQ ID NO: 16.

According to a third aspect, the present invention is directed an antibody that binds to heparin, chondroitin-2,6-sulfate with 20 degrees of polymerization (dp20), and dermatan sulfate, wherein the antibody comprises the amino acid sequence of SEQ ID NO: 17, SEQ ID NO: 18, and/or SEQ ID NO: 22.

According to a fourth aspect, the present invention is directed to an antibody that binds to heparin, chondroitin-2,6-sulfate with 20 degrees of polymerization (dp20), and dermatan sulfate, wherein the antibody comprises an amino acid sequence of SEQ ID NO: 19, and/or SEQ ID NO: 20, and/or SEQ ID NO: 23.

According to a fifth aspect, the present invention is directed to anti-GAG antibody comprising a methionine in the penultimate position of the VHCDR1 according to the Kabat numbering system and that binds to any combination of 2 or more of the following 4 classes of glycosaminoglycan (GAG): heparin/heparan sulfate, chondroitin sulfate/dermatan sulfate, keratan sulfate, and hyaluronic acid

According to a sixth aspect, the present invention is directed to anti-GAG antibody comprising the amino acid sequence VKG at the C-terminal end of the VHCDR2 according to the Kabat numbering system and that binds to any combination of 2 or more of the following 4 classes of glycosaminoglycan (GAG): heparin/heparan sulfate, chondroitin sulfate/dermatan sulfate, keratan sulfate, and hyaluronic acid.

According to a seventh aspect, the present invention is directed to anti-GAG antibody comprising the amino acid sequence YLA at the C-terminal end of the VLCDR1 according to the Kabat numbering system and that binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, the anti-GAG antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an eighth aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid and that does not also bind to Neu5Gc-containing gangliosides.

According to a ninth aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid that does not also bind to Neu5Gc-containing gangliosides.

According to a tenth aspect, the present invention is directed to the antibody as described herein for use in killing cancer cells.

According to an embodiment, the antibody as described herein is for use in killing cancer cells ex vivo.

According to an eleventh aspect, the present invention is directed to the antibody as described herein for use in directly killing cancer cells.

According to a twelfth aspect, the present invention is directed to a pharmaceutical composition comprising the antibody as described herein and a pharmaceutically acceptable excipient or diluent.

According to a thirteenth aspect, the present invention is directed to a pharmaceutical composition comprising the antibody as described herein and a pharmaceutically acceptable excipient or diluent.

According to a fourteenth aspect, the present invention is directed to a pharmaceutical composition comprising an anti-GAG antibody and a pharmaceutically acceptable excipient or diluent.

According to a fifteenth aspect, the present invention is directed to a pharmaceutical composition comprising an anti-GAG antibody and a pharmaceutically acceptable excipient or diluent.

According to a sixteenth aspect, the present invention is directed to a method of treating cancer in a subject in need thereof. The method comprises administering a therapeutically effective amount of the antibody as described herein, or the pharmaceutical composition as described herein, to the subject.

According to an embodiment, the cancer aberrantly expresses a membrane-tethered heparan sulfate proteoglycan.

According to an embodiment, the membrane-tethered heparan sulfate proteoglycan is syndecan-1, syndecan-2, syndecan-3, syndecan-4, glypican-1, glypican-2, glypican-3, glypican-4, glypican-5, glypican-6, glypican-7, glypican-8, or any combination thereof.

According to an embodiment, the method further comprises administering a chemotherapeutic agent to the subject.

According to an embodiment, the method further comprises administering another anti-cancer agent to the subject.

According to a seventeenth aspect, the present invention is directed to the antibody or the pharmaceutical composition as described herein, for use in purging cancer cells from an autologous stem cell transplant (ASCT) or an autologous bone marrow transplant (ABMT).

According to an eighteenth aspect, the present invention is directed to the antibody or the pharmaceutical composition as described herein, for use in purging multiple myeloma, Hodgkin's lymphoma or neuroblastoma cells from an autologous stem cell transplant (ASCT) or autologous bone marrow transplant (ABMT).

According to a nineteenth aspect, the present invention is directed to the antibody or the pharmaceutical composition as described herein, for use in purging multiple myeloma, Hodgkin's lymphoma or neuroblastoma cells from an autologous stem cell transplant (ASCT) or autologous bone marrow transplant (ABMT).

According to a twentieth aspect, the present invention is directed to the antibody or the pharmaceutical composition as described herein, for use in autologous stem cell transplantation (ASCT) or autologous bone marrow transplantation (ABMT).

According to an embodiment, the antibody or pharmaceutical composition is for purging cancer cells from an autologous stem cell transplant (ASCT) or an autologous bone marrow transplant (ABMT).

According to an embodiment, the cancer cells are from multiple myeloma, Hodgkin's lymphoma or neuroblastoma.

According to a twenty first aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid for use in purging an autologous stem cell transplant (ASCT) or autologous bone marrow transplant (ABMT) of cancer cells that express a membrane-tethered heparan sulfate proteoglycan (HSPG).

According to an embodiment, the cancer cells are from multiple myeloma, Hodgkin's lymphoma, neuroblastoma or any other cancer in a subject that could benefit from an autologous stem cell transplantation (ASCT) or autologous bone marrow transplantation (ABMT).

According to a twenty second aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid for use in purging an autologous stem cell transplant (ASCT) or autologous bone marrow transplant (ABMT) of hematopoietic stem cells (HSC).

According to a twenty third aspect, the present invention is directed to the antibody or the pharmaceutical composition as described herein, for use in purging an autologous stem cell transplant (ASCT) or autologous bone marrow transplant (ABMT) of hematopoietic stem cells (HSC).

According to a twenty fourth aspect, the present invention is directed to a method of killing cancer cells comprising exposing cancer cells to an effective amount of an anti-GAG antibody, wherein said antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, said antibody does not bind to Neu5Gc-containing gangliosides.

According to an embodiment, the anti-GAG antibody is polyspecific.

According to an embodiment, the method is for directly killing cancer cells.

According to a twenty fifth aspect, the present invention is directed to a method of performing an autologous stem cell transplantation (ASCT) or autologous bone marrow transplantation (ABMT), the method comprising: exposing the hematopoietic stem cells (HSC) of a subject to an anti-GAG antibody, wherein said antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, the hematopoietic stem cells (HSC) are contaminated with, or presumed to be contaminated with, cancer cells.

According to an embodiment, the subject has been diagnosed with cancer.

According to an embodiment, the cancer is multiple myeloma, Hodgkin's lymphoma or neuroblastoma.

According to an embodiment, exposing the autologous hematopoietic stem cells (HSCs) to the anti-GAG antibody kills cancer cells, thereby purging the cancer cells from the autologous HSCs of the subject.

According to an embodiment, the anti-GAG antibody is the antibody as defined in any one of claims 1-26.

According to a twenty sixth aspect, the present invention is directed to the antibody or the pharmaceutical composition as described herein, for use in purging cancer cells and/or hematopoietic stem cells (HSC) from an autologous stem cell transplant (ASCT) or an autologous bone marrow transplant (ABMT).

According to an embodiment, the cancer cells are from multiple myeloma, Hodgkin's lymphoma, neuroblastoma or any other cancer in a subject that could benefit from an autologous stem cell transplantation (ASCT) or autologous bone marrow transplantation (ABMT).

### BRIEF DESCRIPTION OF THE DRAWINGS

Other and further aspects and advantages of the present invention will be better understood upon the reading of the illustrative embodiments about to be described or will be indicated in the appended claims, and various advantages not referred to herein will occur to one skilled in the art upon employment of the invention in practice. Having thus generally described the nature of the invention, reference will now be made to the accompanying drawings, showing by way of illustration, embodiments thereof, and in which:

FIG. 1 shows a schematic of the following GAG structures used in the microarrays: hyaluronic acid, heparin, chondroitin sulfate AC, and chondroitin sulfate D.

FIG. 2 shows a schematic of the following GAG structures used in the microarrays: dermatan sulfate, heparan sulfate (low and intermediate sulfation), heparan sulfate (high sulfation), and keratin sulfate.

FIG. 3 shows a schematic of the heparan sulfate (HS) structures used in the microarrays.

FIG. 4 shows the GAG binding profile of ICLX-1 scFv at a concentration of 50 μg/mL (top) and 10 μg/mL (bottom), RFU: relative fluorescent unit.

FIG. 5 shows the GAG binding profile of ICLX-2 scFv at a concentration of 50 μg/mL (top) and 10 μg/mL (bottom), RFU: relative fluorescent unit.

FIG. 6 shows the comparison of GAG binding profiles of ICLX-1 scFv (lighter shade) and ICLX-2 scFv (darker shade) at a concentration of 10 μg/mL.

FIG. 7 shows the HS binding profile of ICLX-1 scFv at a concentration of 50 μg/mL (top) and 10 μg/mL (bottom), RFU: relative fluorescent unit.

FIG. 8 shows the HS binding profile of ICLX-2 scFv at a concentration of 50 μg/mL (top) and 10 μg/mL (bottom), RFU: relative fluorescent unit.

FIG. 9 shows the GAG binding profile of ICLX-3 scFv at a concentration of 50 μg/mL (top) and 10 μg/mL (bottom), RFU: relative fluorescent unit.

FIG. 10 shows the comparison of GAG binding profiles of ICLX-1 scFv (orange, left bars) and ICLX-3 scFv (blue, right bars) at a concentration of 10 μg/mL.

FIG. 11 shows the HS binding profile of ICLX-3 scFv at a concentration of 50 μg/mL (top) and 10 μg/mL (bottom), RFU: relative fluorescent unit.

FIG. 12 shows the HS binding profiles of ICLX-3 scFv at pH 7.4 (orange, left bars), pH 6.8 (grey, middle bars), and pH 6.0 (blue, right bars) at a concentration of 50 μg/mL (top) and 2 μg/mL (bottom), RFU: relative fluorescent unit.

FIG. 13 shows the GAG binding profile of ICLX-4 scFv at a concentration of 50 μg/mL (top) and 10 μg/mL (bottom), RFU: relative fluorescent unit.

FIG. 14 shows the GAG binding profile of ICLX-5 scFv at a concentration of 50 μg/mL (top) and 10 μg/mL (bottom), RFU: relative fluorescent unit.

FIG. 15 shows the HS binding profile of ICLX-4 scFv at a concentration of 50 μg/mL (top) and 10 μg/mL (bottom), RFU: relative fluorescent unit.

FIG. 16 shows the HS binding profile of ICLX-5 scFv at a concentration of 50 μg/mL (top) and 10 μg/mL (bottom), RFU: relative fluorescent unit.

FIG. 17 shows the assumed dissociation constant (Kd) between HS024 (HS dp8, 2.5 sulfates/disaccharide) and scFv versions of antibodies ICLX-1 to ICLX-5.

FIG. 18 shows the GAG binding profile of 1HS scFv at a concentration of 2 μg/mL (top) and 0.4 μg/mL (bottom), RFU: relative fluorescent unit.

FIG. 19 shows the HS binding profile of 1HS scFv at a concentration of 10 μg/mL (top) and 2 μg/mL (bottom), RFU: relative fluorescent unit.

FIG. 20 shows results of a CellTiter-Glo® assay of RPMI 8226 cells after 2 days (top) or 4 days (bottom) in culture with different concentrations of ICLX-1 IgG1 (circles) or negative control IgG (triangles). Luminescence is an indicator of cell viability.

FIG. 21 shows results of a CellTiter-Glo® assay of A375 cells after 2 days (top) or 4 days (bottom) in culture with different concentrations of ICLX-1 IgG1 (circles) or negative control IgG (triangles). Luminescence is an indicator of cell viability.

FIG. 22 shows results of a CellTiter-Glo® assay of BxPC3 cells after 2 days (top) or 4 days (bottom) in culture with different concentrations of ICLX-1 IgG1 (circles) or negative control IgG (triangles). Luminescence is an indicator of cell viability.

FIG. 23 shows results of a CellTiter-Glo® assay of Daudi cells after 2 days (top) or 4 days (bottom) in culture with different concentrations of ICLX-1 IgG1 (circles) or negative control IgG (triangles). Luminescence is an indicator of cell viability.

FIG. 24 shows results of a CellTiter-Glo® assay of (A) A375 cells after 4 days in culture with different concentrations of ICLX-3 scFv (triangles) or 1HS IgG1 (circles); and (B) A375 cells after 4 days of culture with different concentrations of ICLX-1 IgG4 (red triangles), ICLX-1 scFv (green triangles), IgG4 isotype control (blue circles) or a negative control scFv (purple triangles). Luminescence is an indicator of cell viability.

FIG. 25 shows results of a CellTiter-Glo® assay of HepG2 cells in up to 5 days in culture in complete media (circles), complete media supplemented with ICLX-1 scFv (blue triangles), serum-free media (octagons), serum-free media supplemented with hepatocyte growth factor (HGF) (squares), and serum-free media supplemented with HGF and ICLX-1 scFv (purple triangles). Luminescence is an indicator of cell viability.

FIG. 26 shows results of a CellTiter-Glo® assay of RPMI 8226 cells in up to 5 days in culture in complete media (circles), complete media supplemented with ICLX-1 scFv (blue triangles), serum-free media (octagons), serum-free media supplemented with hepatocyte growth factor (HGF) (squares), and serum-free media supplemented with HGF and ICLX-1 scFv (purple triangles). Luminescence is an indicator of cell viability.

FIG. 27 shows brightfield microscope images of RPMI 8226 cells 4 days after treatment with different concentrations of ICLX-1 scFv using the 40× objective of an inverted Olympus CKX53 phase contrast microscope.

FIG. 28 shows the flow gating strategy used to perform the flow cytometry experiments on the RPMI 8226 cells depicted in FIG. 27 to define dead, apoptotic, and live cells using the Annexin/7-AAD Apoptosis kit.

FIG. 29 shows the flow cytometry results from the RPMI 8226 cells of FIGS. 27 and 28 wherein the dead cells indicated by the arrows lost normal forward scatter (FSC)/side scatter (SSC).

FIG. 30 shows the flow cytometry results from the RPMI 8226 cells of FIGS. 29-31 demonstrating a majority of dead cells undergoing necrotic death compared to very few Annexin V single positive cells undergoing apoptotic cell death.

FIG. 31 shows the quantification of the flow cytometry results from the RPMI 8226 cells of FIGS. 29-32 with (A) dead cell percentage and (B) normalized viable cells as a percentage of non-treated cells after treatment with different concentrations of ICLX-1 scFv.

FIG. 32 shows the percentage cell viability of MIA PaCa-2 cells grown in 3D tumor spheroids treated with (A) different concentrations of ICLX-1 scFv, or (B) different concentrations of staurosporine for 6 days using the CellTiter-Glo® Assay.

FIG. 33 shows the percentage cell viability of PANC-1 cells grown in 3D tumor spheroids treated with (A) different concentrations of ICLX-1 scFv, or (B) different concentrations of staurosporine for 6 days using the CellTiter-Glo® Assay.

FIG. 34 shows the percentage cell viability of A549 cells grown in 3D tumor spheroids treated with (A) different concentrations of ICLX-1 scFv, or (B) different concentrations of staurosporine for 6 days using the CellTiter-Glo® Assay.

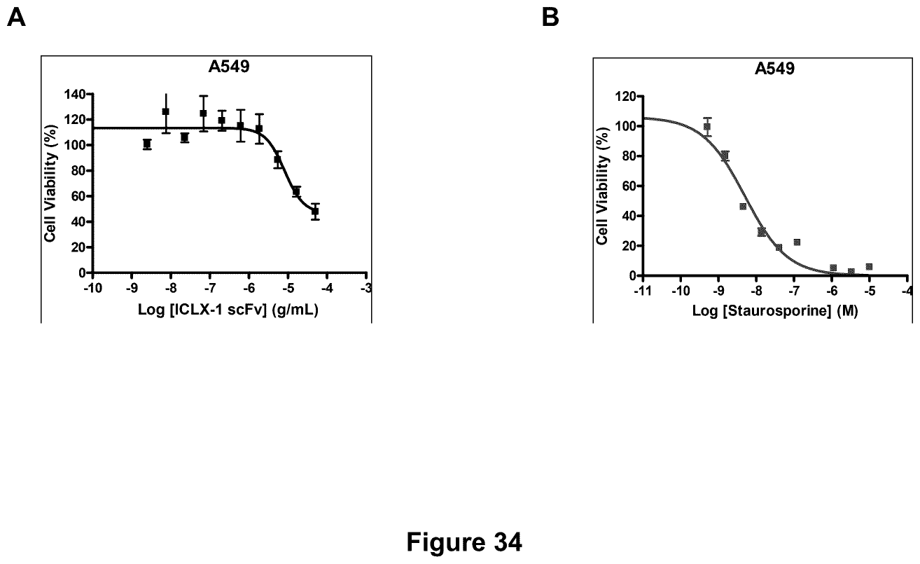

FIG. 35 shows the GAG binding profile of AO4B08 scFv at a concentration of 100 μg/mL (top) and 50 μg/mL (bottom), RFU: relative fluorescent unit.

FIG. 36 shows the GAG binding profile of AO4B08 scFv at a concentration of 25 μg/mL (top) and 12.5 μg/mL (bottom), RFU: relative fluorescent unit.

FIG. 37 shows the GAG binding profile of AO4B08 scFv at a concentration of 6.25 μg/mL (top) and 3.125 μg/mL (bottom), RFU: relative fluorescent unit.

FIG. 38 shows the GAG binding profile of AO4B08 scFv at a concentration of 1.56 μg/mL (top) and 0.78 μg/mL (bottom), RFU: relative fluorescent unit.

FIG. 39 shows the GAG binding profile of AO4B08 scFv at a concentration of 0.39 μg/mL (top) and 0.195 μg/mL (bottom), RFU: relative fluorescent unit.

FIG. 40 shows the GAG binding profile of AO4B08 scFv at a concentration of 0.0975 μg/mL (top) and 0.049 μg/mL (bottom), RFU: relative fluorescent unit.

FIG. 41 shows the GAG binding profile of the negative control 1 without AO4B08 scFv (top) and of the negative control 2 without AO4B08 scFv and without the anti-6×His Ab (bottom), RFU: relative fluorescent unit.

FIG. 42 shows the HS binding profile of AO4B08 scFv at a concentration of 100 μg/mL (top) and 50 μg/mL (bottom), RFU: relative fluorescent unit.

FIG. 43 shows the HS binding profile of AO4B08 scFv at a concentration of 25 μg/mL (top) and 12.5 μg/mL (bottom), RFU: relative fluorescent unit.

FIG. 44 shows the HS binding profile of AO4B08 scFv at a concentration of 6.25 μg/mL (top) and 3.125 μg/mL (bottom), RFU: relative fluorescent unit.

FIG. 45 shows the HS binding profile of AO4B08 scFv at a concentration of 1.56 μg/mL (top) and 0.78 μg/mL (bottom), RFU: relative fluorescent unit.

FIG. 46 shows the HS binding profile of AO4B08 scFv at a concentration of 0.39 μg/mL (top) and 0.195 μg/mL (bottom), RFU: relative fluorescent unit.

FIG. 47 shows the HS binding profile of AO4B08 scFv at a concentration of 0.0975 μg/mL (top) and 0.049 μg/mL (bottom), RFU: relative fluorescent unit.

FIG. 48 shows the HS binding profile of the negative control 1 without AO4B08 scFv (top) and of the negative control 2 without AO4B08 scFv and without the anti-6×His Ab (bottom), RFU: relative fluorescent unit.

FIG. 49 shows the GAG binding profile of 3HS scFv at a concentration of 50 μg/mL (top) and 10 μg/mL (bottom), RFU: relative fluorescent unit.

FIG. 50 shows the GAG binding profile of 3HS scFv at a concentration of 2 μg/mL (top) and 0.4 μg/mL (bottom), RFU: relative fluorescent unit.

FIG. 51 shows the GAG binding profile of 3HS scFv at a concentration of 0.08 μg/mL (top) and 0.016 μg/mL (bottom), RFU: relative fluorescent unit.

FIG. 52 shows the GAG binding profile of 3HS scFv at a concentration of 0.0032 μg/mL (top) and of the negative control 1 without 3HS scFv, RFU: relative fluorescent unit.

FIG. 53 shows the flow gating strategy used to define dead, apoptotic, and live cells in the flow cytometry experiments. RPMI 8226 cells were treated for 4 days with different concentrations of AO4B08 scFv, 1HS scFv, or 3HS scFv.

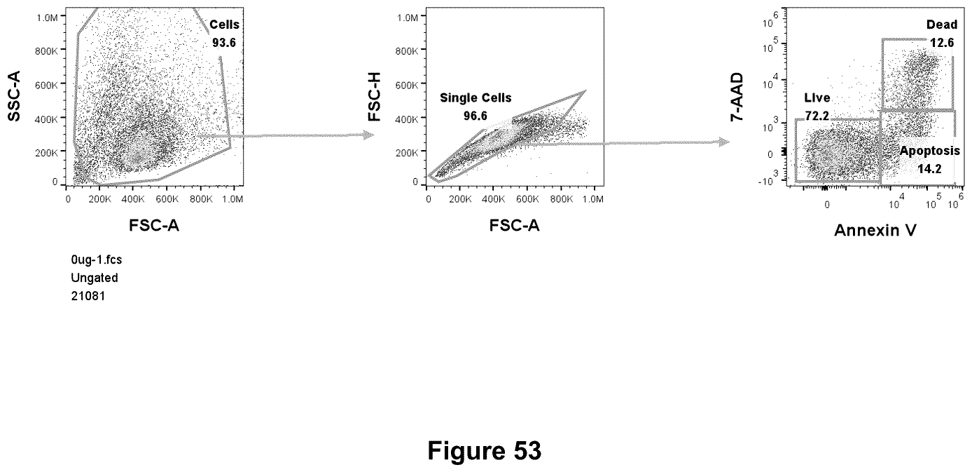

FIG. 54 shows the quantified flow cytometry results for the RPMI 8226 cells in FIG. 53 as (A) percentage cell viability, (B) normalized viable cells as a percentage of non-treated cells, (C) percentage cell death, and (D) percentage apoptotic cells. RPMI 8226 cells were treated for 4 days with different concentrations of AO4B08 scFv, 1HS scFv, or 3HS scFv.

FIG. 55 shows the percentage cell viability of SK-N-FI neuroblastoma cells grown in 3D tumor spheroids treated with (A) different concentrations of ICLX-1 scFv, or (B) different concentrations of staurosporine for 6 days using the CellTiter-Glo® Assay.

### DETAILED DESCRIPTION

Novel antibodies that bind to more than one class of glycosaminoglycan, and in particular, to heparan sulfate side chains of proteoglycans will be described hereinafter. By interfering with heparan sulfate's function as a co-receptor for growth factor binding to canonical receptors, the antibodies of the present disclosure have a direct, antiproliferative and cytotoxic effect on cancer cells, analogous to serum starvation. Although the invention is described in terms of specific illustrative embodiments, it is to be understood that the embodiments described herein are by way of example only and that the scope of the invention is not intended to be limited thereby.

The terminology used herein is in accordance with definitions set out below.

By “about”, it is meant that the value can vary within a certain range depending on the margin of error of the method or device used to evaluate or measure. A margin of error of 10% is generally accepted.

As used herein, the expression “associated with”, when used in the context of a glycosaminoglycan and a protein core, signifies that the glycosaminoglycan is covalently bound to the protein core to form a proteoglycan. For example, “heparan sulfate in association with syndecan-1” means that the heparan sulfate is covalently bound to the core protein, syndecan-1.

“Cancer” refers to a disease of malignantly transformed cells.

“High sulfation glycosaminoglycan” means a glycosaminoglycan with greater than two sulfate groups per disaccharide unit. For the sake of clarity, chondroitin-4-sulfate (also called chondroitin sulfate A) is not a high sulfation glycosaminoglycan.

“Low sulfation heparan sulfate” means an heparan sulfate with less than one sulfate groups per disaccharide unit.

“Intermediate sulfation heparan sulfate” means an heparan sulfate with about two sulfate groups per disaccharide unit.

“Isolated heparan sulfate” refers to either a heparan sulfate molecule that is not attached to a protein core, or one that has been bound to a glycan microarray for detection purposes. Glycan microarrays comprise glycan molecules with amino linkers that have been printed onto N-hydroxysuccinimide (NHS)-activated glass microscope slides via covalent amide linkages.

“Tumor” can mean any mass, but in the present context, one that is neoplastic and malignant in nature.

“Polyspecific” in the context of the present disclosure means reacting with more than one class of GAG. Structurally and biochemically, there are 4 classes of GAG: Hep/HS, CS/DS, KS, and HA.

“Direct killing” in the context of the present disclosure means necrotic cell death in the absence of complement or natural killer (NK) cells.

An “anti-GAG antibody” in the context of the present disclosure is an antibody that specifically binds to an isolated glycosaminoglycan (GAG), as opposed to an antibody that only binds to a GAG that itself is covalently bound to a core protein.

The following abbreviations are used throughout the present disclosure are defined hereinbelow for convenience:
2OST: 2-O-sulfotransferase
30ST: 3-O-sulfotransferase
6OST: 6-O-sulfotransferase
Ab: antibody
ABMT: autologous bone marrow transplantation
ACAN: aggrecan
Ag: antigen
APTT: activated partial thromboplastin time
ASCT: autologous stem cell transplantation
BCAN: brevican
BSA: bovine serum albumin
CS: chondroitin sulfate
CSA: chondroitin-4-sulfate, or chondroitin sulfate A
CSC: chondroitin-6-sulfate, or chondroitin sulfate C
CSD: chondroitin-2,6-sulfate, or chondroitin sulfate D
CSE: chondroitin-4,6-sulfate, or chondroitin sulfate E
CSPG: chondroitin sulfate proteoglycan
CSPG4: Chondroitin Sulfate Proteoglycan 4
CW motif: Cardin Weintraub motif
ddPCR: droplet digital polymerase chain reaction
dp: degree of polymerization
DS: dermatan sulfate
DSPG: dermatan sulfate proteoglycan
EC: endothelial cell
ELISA: enzyme-linked immunosorbent assay
EOC: epithelial ovarian cancer
EpCAM: epithelial cell adhesion molecule
FACS: fluorescence activated cell sorting
Fc: fragment crystallizable
FCM: flow cytometry
FGF: fibroblast growth factor
FGF2 or bFGF: basic fibroblast growth factor
FGFR: fibroblast growth factor receptor
GalNac: N-acetyl galactosamine
GBP: glycosaminoglycan-binding protein
GlcA: glucuronic acid
GLCE: D-glucuronyl C5-epimerase
GlcNAc: N-acetyl glucosamine
GPC: glypican
GPI: glycosyl-phosphadityl-inositol
gpnmb: transmembrane glycoprotein NMB
HA: hyaluronic acid
HCC: hepatocellular carcinoma
Hep: heparin
hFc: human fragment crystallizable
HGF: hepatocyte growth factor
HS: heparan sulfate
HSC: hematopoietic stem cell
Hsp: heat shock protein
HSPG: heparan sulfate proteoglycan
IdoA: L-iduronic acid
Ig: immunoglobulin
IgG: immunoglobulin G
IgM: immunoglobulin M
IHC: immunohistochemistry
IL-8: interleukin-8
i.p.: intraperitoneally
KS: keratan sulfate
LMWH: low molecular weight heparin
mAb: monoclonal antibody
MM: multiple myeloma
MTT: 3-(4,5-dimethylthiazol-2-yl)-2,5-diphenyltetrazolium bromide
MTS: (3-(4,5-dimethylthiazol-2-yl)-5-(3-carboxymethoxyphenyl)-2-(4-sulfophenyl)-2H-tetrazolium)
NCAN: neurocan
NDST: N-deacetylase/N-sulfotransferase
Neu5Gc: N-glycolylneuraminic acid
NeuGcGM3: N-glycolylneuraminic GM3 ganglioside
nTPM: normalized transcripts per million
PBMC: peripheral blood mononuclear cell
PDGF: platelet-derived growth factor
PG: proteoglycan
pHe: extracellular pH
PI: propidium iodide
s.c.: subcutaneously
scFv: single-chain variable fragment
SCID: subacute combined immunodeficient
SCLC: small cell lung cancer
sdAb: single domain antibody
SDC: syndecan
TGFβ1: transforming growth factor beta 1
TME: tumor microenvironment
VCAN: versican
VEGF: vascular endothelial growth factor
VGF-A: non-acronymic granin-like neuropeptide precursor
V_{H}: variable region heavy chain
V_{L}: variable region light chain

The interaction between growth factors and their canonical receptors typically involves specific binding sites on both molecules. These binding sites are often complementary in shape and charge, ensuring a precise and selective interaction. Examples of canonical receptor families include receptor tyrosine kinases (RTKs) and cytokine receptors.

GAGs are long, linear carbohydrate chains that are attached to core proteins, forming proteoglycans (PGs). They are abundant components of the extracellular matrix and are also present on the cell surface, where they're referred to as membrane-tethered PGs. GAGs, such as heparan sulfate (HS), carry a negative charge due to sulfate and carboxylate groups along their chains. This negative charge allows them to interact electrostatically with positively charged regions on growth factors.

The interaction between growth factors (e.g., FGF, VEGF, TGFβ1, HGF) and GAGs, in particular HS, enhances growth factor binding and signaling in several ways:
a) Co-receptor Function: GAGs, especially HS, can act as co-receptors alongside canonical receptors on the cell surface. They facilitate the binding of growth factors to their canonical receptors, enhancing the stability and specificity of the interaction.
b) Concentration and Presentation: GAGs can concentrate growth factors in the extracellular matrix and on the cell surface, creating a localized reservoir of signaling molecules. This can lead to a more potent and sustained cellular response to the growth factor.
c) Modulation of Signaling: The presence of GAGs can modulate the signaling activity of growth factors. They can affect the binding affinity between growth factors and canonical receptors, influence receptor dimerization, and guide the orientation of growth factor-receptor complexes, all of which impact downstream signaling pathways.
d) Cellular Responses: GAGs can influence cellular responses to growth factors by fine-tuning their availability and interaction dynamics. They contribute to processes such as cell adhesion, migration, and tissue development.

It's important to note that the interactions between growth factors, canonical receptors, and GAGs are often interconnected and interdependent. The presence of GAGs can enhance the binding of growth factors to canonical receptors and promote effective signal transduction, ultimately regulating a wide range of cellular processes.

One major difference between the growth factor-canonical receptor interaction and the growth factor-GAG interaction is that the former involves a high precision, ‘lock and key’ molecular mechanism, whereas the latter is predominantly electrostatic in nature (Xu and Esko, 2014). This has prompted some groups to employ sophisticated computational, structural, and biochemical strategies to seek out the few, highly specific, so-called druggable protein-GAG interactions (Sarkar and Desai, 2015). The strategy employed herein takes the opposite approach, emphasizing the utility of interfering with the more common form of protein-GAG interaction by means of antibodies that bind electrostatically to a wide range of sulfated GAGs.

Each of the human antibodies described in the present disclosure binds to more than one class of GAG and often to more than one degree of polymerization (dp) within a given class. They do not recognize a specific motif; instead, they bind to GAGs with a high negative charge density. In this regard, they share the same binding mechanism as most growth factor-GAG interactions and are thus capable of effectively mitigating aberrant growth factor signaling in a wide range of cancer cells.

GAGs, particularly Hep, can also increase the efficiency of chemotherapy drugs by dissolving clots which are associated with tumor tissue, thereby reducing interstitial pressure and increasing drug bioavailability. In one study, patients with small cell lung cancer (SCLC) benefited from a subcutaneous Hep injection before the onset of chemotherapy (Lebeau, et al., 1994); an observation which was subsequently confirmed by clinical trials (Altinbas, et al. 2004; Altinbas, et al. 2014; Lebeau, et al. 2011). And in a non-randomized clinical study of pancreatic cancer patients, treatment with low molecular weight heparin (LMWH) in combination with gemcitabine and cisplatin led to a significant improved survival rate (Icli, et al., 2007). LMWH has also been shown to enhance the uptake of paclitaxel and doxorubicin into lung adenocarcinoma xenografts and to augment the efficacy of chemotherapy drugs on tumor xenografts (Phillips, et al. 2011).

GAGs may be found on essentially all cell surfaces and in the extracellular matrix, and play important functional and structural roles. GAG complexity and functional specificity is the result of several post-translational modifications, including the site and degree of sulfation, as well as acetylation; the type of linkages between saccharide units within the chain; and a variety of sequence variations, since GAGs are not simple repeating disaccharides. As a result, GAGs contain highly specific biochemical information which accounts for their functional and organ specificity. They are among the most information-rich molecules in nature, second only to the nucleic acids; however, unlike the latter, GAG complexity is not based on any template but rather, on a series of enzymatic cascades.

GAGs may be further subdivided into sulfated (Hep, HS, CS, DS, and KS) or non-sulfated (HA) molecules. Some GAGs (HS, CS, DS, and KS) are associated with a protein core, in which case they form proteoglycans (PGs), while others (Hep and HA) exist primarily as isolated molecules.

HA is the simplest GAG, and consists of unsulfated [GlcA(β1→3)GlcNAc(β1→4)] dimers.

CS is built around a backbone consisting of [GlcA(β1→3)GalNAc(β1→4)] dimers, with a variable sulfation pattern of both GlcA and GalNAc.

DS is built around a backbone consisting of [GlcA/IdoA(β1→3)GalNAc(β1→4)] dimers, with a variable sulfation pattern of both IdoA and GalNAc.

KS is built around a backbone consisting of [Gal(β1→4)GalNAc(β1→3)] dimers, with a variable sulfation pattern of both Gal and GlcNAc.

Hep is built around a backbone consisting of [GlcA/IdoA,2S(β1→4)GlcNS/GlcNAc(α1→4)] dimers, in which there is a high degree of sulfation. Although the backbone of Hep somewhat resembles the backbone of HA, the linkages between GlcNAc and GlcA are different.

HS, which is structurally very similar to Hep, is also built around a backbone of [GlcA/IdoA(β1→4)GlcNS/GlcNAc(α1→4)] dimers, but the disaccharide units are organized into distinct sulfated (S) and N-acetylated (NA) domains, as well as transition (NA/NS) zones. The S-domains are responsible for most of the polysaccharide's biological activities, and they vary over different organs, developmental stages, and pathologies.

The synthesis of HS is a complex process involving multiple specialized enzymes. The process of chain elongation is initiated from a tetrasaccharide (GlcA-Gal-Gal-Xyl) that is attached to a core protein via a serine (Ser) residue. Further elongation is promoted by a hetero-oligomeric complex formed by exostosin 1/exostosin 2 (EXT1/EXT2), which are responsible for building the polysaccharide backbone with a repeating unit of [GlcA(β1→4)GlcNAc(α1→4)]. The backbone is then modified by N-deacetylase/N-sulfotransferase (NDST), responsible for N-deacetylation and N-sulfation of selected GlcNAc residues, D-glucuronyl C5-epimerase (GLCE) responsible for epimerization of selected GlcA moieties to IdoA, 2-O-sulfotransferase (2OST) responsible for 2-O-sulfation of selected IdoA residues, 6-O-sulfotransferase (6OST) responsible for 6-O-sulfation, and finally (but rarely) 3-O-sulfotransferase (30ST) responsible for 3-O-sulfation. Further modifications of HS structure may also take place post-synthetically, through the action of the 6-O-endosulfatases Sulf-1 and Sulf-2, and heparanase. The substrate specificities of these biosynthetic enzymes dictate the structures of HS products including sulfation levels, the frequency of IdoA units, and the length of the polysaccharides. The location of the sulfate groups and IdoA units, in turn, play a crucial role in determining the binding and functions of HS. The current consensus is that N- and 2-O-sulfated units are involved in low specificity binding, 6-O-sulfated units in intermediate specificity binding, and 3-O-sulfated and N-unsubstituted units are responsible for high specificity binding (Marques, et al. 2021).

Examples of full-time membrane-tethered core proteins that form heparan sulfate proteoglycans (HSPGs) are the syndecans (SDC1-4) and glypicans (GPC1-6). Another name for SDC1 is CD138. SDC1 and SCD3 can also act as chondroitin sulfate proteoglycans (CSPGs; Sarrazin, et al. 2011). Part-time membrane-tethered core proteins that form HSPGs are betaglycan (TGFBR3) and neuropilin-1 (NRP1). Extracellular matrix-localized HSPGs are perlecan (HSPG2), agrin (AGRN), and collagen type XVIII (COL18A1). Serglycin (SRGN) is a PG found in secretory vesicles. Many types of cancer cells and their associated stroma exhibit increased expression of HSPGs. HSPGs play a crucial role in tumor growth, angiogenesis, metastasis, and evasion of the immune system. They help cancer cells interact with growth factors, cytokines, and extracellular matrix components. For a review of antibodies that bind to HSPG core proteins (e.g., SDC1 or GPC1), see Onyeisi, et al. (2020).

Examples of core proteins that form CSPGs are members of the lectican family: aggrecan (ACAN), versican (VCAN), neurocan (NCAN), and brevican (BCAN). These are found in the extracellular matrix (ECM). BCAN may be found in both the ECM and on the cell membrane. CSPG4 is a core protein of special interest because it is a transmembrane PG originally identified as a highly immunogenic tumor antigen on the surface of melanoma cells (Price, et al. 2011). Some cancer types exhibit increased CSPG expression, notably CSPG4.

Examples of DSPGs are decorin (DCN), biglycan (BGN), thrombomodulin (TMBD), endocan, epiphycan (EPYC), and versican (VCAN) (Trowbridge JM and Gallo, 2002). DSPGs have been associated with certain types of cancer, and their overexpression can play a role in tumor progression. They may influence cell adhesion, migration, and the tumor microenvironment (TME).

Finally, and for the sake of completeness, CD44v3 is a non-kinase transmembrane glycoprotein that forms a PG with HA. Activation of CD44v3 by HA stimulates cell signaling pathways that induce cell proliferation, increases cell survival, modulates cytoskeletal changes, and enhances cellular motility (Chen, et al., 2018).

Most of the biological roles of HSPGs are mediated by their GAG sidechains through binding various protein ligands including cytokines, chemokines, growth factors and receptors, morphogens, proteases, protease inhibitors, and ECM proteins. In basement membranes, HSPGs collaborate with other matrix components to define basement membrane structure and to provide a matrix for cell migration and invasion. HSPG dysregulation may hijack these pathways to confer cellular malignancy leading to tumor development and progression (Yang and Wang, 2023). Comparative studies have demonstrated aberrant expression of several genes encoding HS biosynthesis machinery in cancer. Transcriptomic and immunohistochemical analyses have been performed in breast and colorectal tumor samples and indicated significant variations in the expression and tissue distribution of several enzymes involved mostly in HS epimerization and sulfation (Marques, et al. 2021).

Sterically interfering with and blocking GAG side chains on membrane-tethered HSPGs raises the prospect of both mitigating HSPG dysregulation and attenuating growth factor binding to cancer cells. One means of achieving these dual goals is by means of antibodies and antibody fragments that bind sulfated GAGs, in particular, HS.

Antibodies are Y-shaped protein molecules that comprise hypervariable, variable, and constant regions. Each antibody unit includes a heavy and light chain. Each chain contains a single variable region, and each variable region contains three hypervariable regions. It is the hypervariable regions that ultimately confer specificity upon the antibody, although the entire heavy and light chain structure may be required for determining the final 3-dimensional shape of the antibody.

Antibodies may be used to cause tumor cell death through the blockade of growth factor signaling via a direct mechanism. For example, epidermal growth factor receptor (EGFR) is overexpressed by many different cancers, and signaling via EGFR leads to tumor cell proliferation, migration, and invasion. Cetuximab, which is an anti-EGFR mAb, induces apoptosis in tumor cells by blocking ligand binding and receptor dimerization (Li, et al, 2005; Patel, et al, 2009). Human epidermal growth factor receptor 2 (HER2) is a tyrosine kinase receptor that is overexpressed in many cancers but primarily ovarian and breast carcinomas (Slamon, et al. 1989). It is distinct from EGFR in that it has no known ligand and instead hetero-dimerizes with other growth factor receptors to enhance their activation (Chen, et al, 2003). Antibodies targeting HER2 therefore achieve signaling perturbation by inhibiting hetero-dimerization and internalization. Trastuzumab was the first FDA approved anti-HER2 mAb and remains a vital component of treatments for HER2-amplified breast cancer. Interestingly, HS has been shown to mediate the effect of trastuzumab in breast cancer cells (Suarez, et al. 2013). For a review of monoclonal antibodies in cancer therapy, see Zahavi D and Weiner L (2020).

Using techniques that are well-known to the person skilled in the art, antibodies of any class and fragments thereof can be custom produced based only on knowledge of the amino acid sequence of their variable or hypervariable regions.

Most antibodies interact with their antigenic targets with ‘lock and key’ precisions, however, anti-GAG antibodies seldom fit neatly into this paradigm. Currently, there are over 800 biomolecules in the GAG interactome (827 proteins and 5 GAGs; Vallet, et al. 2020), including 932 protein-GAG interactions; most of these interactions are electrostatic and nonspecific in nature, with hydrogen-bonding, van der Waals interactions, and hydrophobic effects accounting for the nonspecific component.

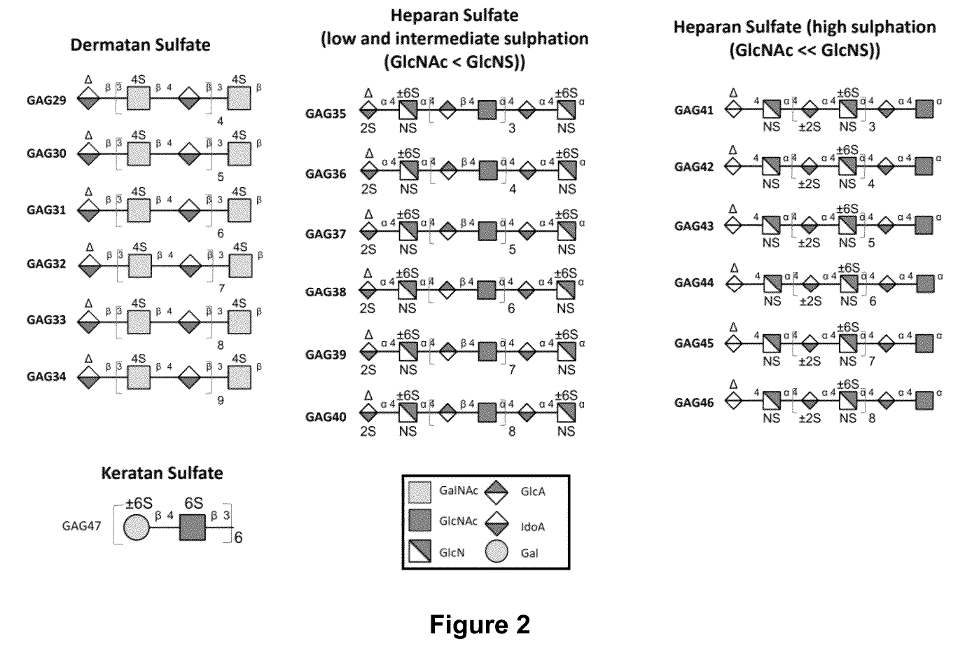

A few specific protein-GAG interactions have been described; however, they constitute a minority (Sarkar and Desai, 2015). The classic example would be the binding of antithrombin III to a unique Hep pentasaccharide comprised of a specific arrangement of sulfate groups and uronic acid epimers (i.e., GlcNAc/NS(6S)-GlcA-GlcNS(3S,6S)-IdoA(2S)-GlcNS(6S); Lindahl, et al, 1980). Binding sites for specific antibody-GAG interactions typically consist of no more than 5 sugars (see FIG. 2 of Kerever and Arikawa-Hirasawa, 2021). This is important because the antibodies of the present disclosure bind to Hep chains containing 30 sugars, HS chains with a minimum of 9 sugars, and in the case of CSD, chains containing no less than 20 sugars. These results are consistent with an electrostatic, as opposed to a ‘lock and key’, binding mechanism.

The antibody-GAG interaction is a sub-category of the protein-GAG interaction. Cardin and Weintraub (1989) proposed 2 consensus sequences for GAG binding proteins (GBPs) that have stood the test of time: ‘XBBXBX’ and ‘XBBBXXBX”, where B and X represent basic and hydropathic residues, respectively. The basic residues are arginine (Arg) and lysine (Lys), and at lower pH values, histidine (His), which becomes increasingly protonated as the pH nears 6.0. Hydropathic (i.e., hydrophobic) amino acids consist of isoleucine (Ile), valine (Val), leucine (Leu), phenylalanine (Phe), cysteine (Cys), methionine (Met), alanine (Ala), glycine (Gly), and threonine (Thr) (Kyte and Doolittle, 1982). Two other amino acids, aspartic acid (Asp) and glutamic acid (Glu), should also be considered when evaluating the binding of an oligopeptide to a GAG, since these are negatively charged and will not interact favorably with a negatively charged GAG.

Cardin-Weintraub (CW) motifs are important determinants of the protein-GAG interaction, but they are not the whole story. Non-ionic interactions (e.g., hydrogen bonds, van der Waals forces, and hydrophobic interactions) as well as secondary and tertiary structures of the basic amino acid residues themselves may also play an important role in the protein-GAG interaction (Margalit, et al., 1993).

In the present invention, the antibody-GAG interaction is viewed through the lens of CW motifs. If any antibody (not just the antibodies of the invention) binds to a GAG, then chances are good that basic amino acid residues and CW-like motifs will be found in the heavy and light chain CDR regions, extending at times even into the adjacent framework regions. Said antibodies may then be intentionally modified by the addition or substitution of specific amino acids with the aim of improving the positive charge density of the CW motif. A good example of this is the human-mouse chimeric antibody chP3R99 (vide infra).

The history of anti-GAG antibodies dates to the early 1990's, with the development of the first murine mAbs to Hep and HS. Along the way, human anti-GAG antibodies were produced, first by chimerization of murine mAbs, then by human-human hybridomas, and finally, using phage display technology. Each of these methods has its own advantages and disadvantages in terms of antibody production and specificity. The NIH Database of Anti-Glycan Reagents (DAGR) (https://dagr.ccr.cancer.gov/) currently lists only 21 antibodies that react with HS, but the list is by no means encyclopedic.

### Murine Monoclonal Antibodies to Heparin and Heparan Sulfate

The antigenicity of GAGs is very weak, thus, there have only been a few reports of the successful production of anti-HS mAbs using conventional murine hybridoma technology.

Straus, et al. (1992) described a murine mAb (ST-1) which was raised against Hep complexed to Salmonella minnesota. Characterization of ST-1 showed that it recognized an epitope in the intact Hep molecule that is present regardless of its source or anticoagulant activity. ST-1 is regarded as the first mAb specific for the intact, unmodified Hep molecule. No cross reactivity of ST-1 was observed with HA, HS, CS, DS, or KS. Selective removal of the N-sulfate groups or N,O-desulfation of Hep strongly reduced the binding of ST-1. Competitive assays of ST-1 binding to Hep immobilized on poly-L-lysine-coated plates using oligosaccharides of different sizes produced by HNO₃ cleave of Hep showed that the minimum fragment required for reactivity of ST-1 is a decasaccharide.

Huhle, et al. (1997) described a murine mAb (H1.18) which was raised following immunization with a Hep-BSA conjugate, prepared by reductive amination. H1.18 was shown to recognize specifically intact Hep and Hep fractions. The lower detection limit for Hep preparations was 100 ng/mL. The smallest disaccharide that was detected by H1.18 was found to be iduronic acid-anhydromannose (i.e., a disaccharide). The GAGs HS, DS, KS, CSA, and CSC did not show any reactivity with mAb H1.18 up to concentrations of 100 mg/mL.

Notwithstanding the many similarities between Hep and HS, antibodies ST-1 and H1.18 bind specifically with the former but not the latter. This is a key feature of these two antibodies. Examples of antibodies with the converse binding preference have also been described.

van den Born, et al. (2005) studied the saccharide epitope structures of four anti-HS mAbs, HepSS1, JM13, JM403, and 10E4, which all recognize distinct HS species as demonstrated by different patterns of immunoreactivity upon staining of embryonic rat and adult human tissues. The epitopes recognized by JM13 and HepSS1 were found almost exclusively in basement membrane HS, whereas JM403 and 10E4 reacted also with cell-associated HS species. The binding of HepSS1, JM403, and 10E4 to HS was dependent on the GlcN N-substitution of the polysaccharide rather than O-sulfation. HepSS1 thus interacted with N-sulfated HS domains, JM403 binding was critically dependent on N-unsubstituted GlcN residues, and 10E4 bound to “mixed” HS domains containing both N-acetylated and N-sulfated disaccharide units. By contrast, JM13 binding seemed to require the presence of 2-O-sulfated glucuronic acid residues.

None of the four anti-HS antibodies studied by van den Born, et al. (2005) bound to native Hep. The authors concluded that “Our results suggest that HepSS1 and JM13 bind to rare saccharide epitopes that occur predominantly in basement membrane HS” and opined that “Detection of HS by monoclonal antibodies is used increasingly in studies addressing the role of the polysaccharide in various biological phenomena and its distribution in cells and tissues. Because of the complex and highly variable structure of HS, such antibodies likely recognize specific epitopes that may be present in some HS species and absent in others instead of recognizing ubiquitous HS structures. This assumption is particularly relevant for antibodies raised by immunization of animals with HS because the most commonly occurring saccharide structures are not likely to elicit an immune response. By contrast, antibodies generated by the phage display technique do not depend on the host immune response and may conceivably bind any HS structure regardless of the frequency of its occurrence in natural HS species. The results by Dennissen, et al. (2002) support this view by demonstrating that many generally occurring saccharide structures found in HS and heparin were recognized by the phage display antibodies.” Indeed, the weak immunogenicity and high biocompatibility of Hep is exemplified by the thousands of patients who receive Hep anticoagulation therapy every year without immunological incident.

### chP3R99: A Rationally Designed Chimeric Antibody with an ‘Ideal’ CW Motif in its VHCDR3

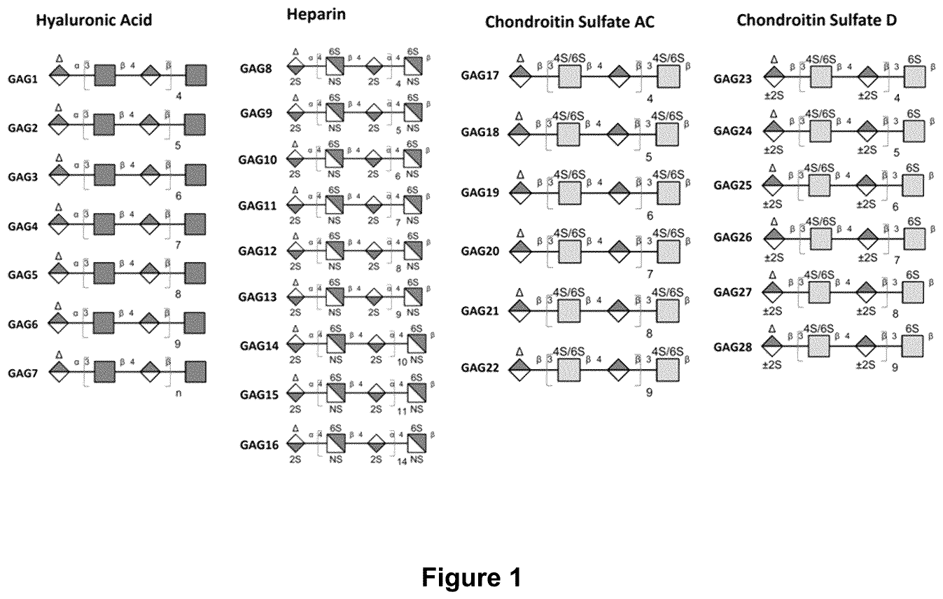

The mouse-human chimeric antibody, chP3R99, is disclosed in WO2010/127642, U.S. Pat. No. 8,470,322 and CA2758944, where it's simply referred to as “chimeric MAb anti-SO3”. It reacts in ELISA to a variety of sulfated GAGs including Hep, HS, CS, and DS; with minimal binding to HA (see FIG. 1 of Soto, et al. 2012).

KABAT indicates amino acid numbering according to Kabat (Kabat et al., Sequences of Proteins of Immunological Interest, 5th Ed. Public Health Service, National Institutes of Health, Bethesda, MD. (1991)). The heavy and light chain CDR sequences (Kabat) of chP3R99, with their corresponding CW motifs, are represented in the Table below. Histidine residues are indicated as B because histidine has a pKa of 6.04 and as the pH falls closer to 6.0, an increasingly larger proportion of histidines will become protonated and hence positively charged, thus favoring the formation of electrostatic interactions with negatively charged sulfate and carboxylate groups of GAGs (see Gandhi and Mancera, 2008). Aspartic acid and glutamic acid residues are marked with 0 because they are negatively charged and interact unfavourably with GAGs.

| chP3R99 | CDR1 | CDR2 | CDR3 |
|---|---|---|---|
| VH | RYSVH | MIWGGGSTDYNSALKS | SGVRRGRAQAWFAY |
| | B--XB | XX-XXX-XO---XXB- | -XXBBXBX-X-XX- |
| VL | KASQDVSTAVA | SASYRYT | QQHYSTPWT |
| | BX--OX-XXXX | -X--B-X | --B--X--X |

An ‘ideal’ CW motif, ‘XBBXBX’ (represented by the amino acid sequence VRRGRA), in the VHCDR3 (Kabat) of antibody chP3R99- can clearly be seen. This is an excellent example of a CW motif occurring in exactly the one location in an antibody molecule where it would be expected to have a GAG-binding function, i.e., in the middle of VHCDR3 (Kabat).

Antibody chP3R99 is a derivative of the murine IgMK mAb, P3, which was generated by immunizing BALB/c mice with N-glycolyl GM3 ganglioside (NeuGcGM3) incorporated into liposomes and selecting against N-glycolylneuraminic acid-containing gangliosides (Vázquez, et al. 1995). P3 reacts with breast carcinomas (Vázquez, et al. 1995) and melanomas (Alfonso, et al. 2002) by IHC; V_{H} and V_{L} sequences for P3 are disclosed in Perez, et al. 2001.

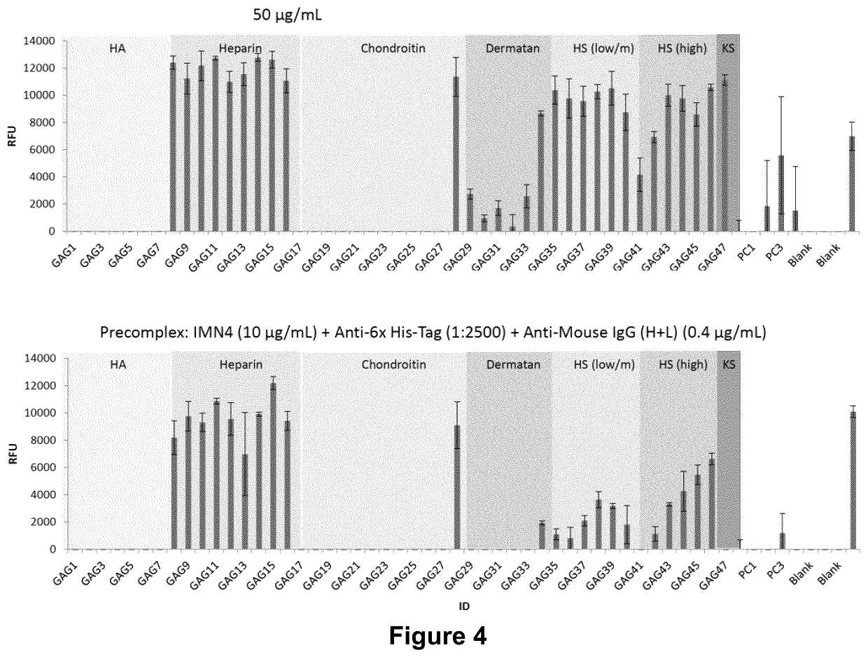

An interesting property of P3 is its ability to elicit a strong anti-idiotypic response when administered in a syngeneic animal model, even in the absence of adjuvants or carrier proteins (see U.S. Pat. No. 6,491,914 and Vázquez, et al. 1998). This prompted Alfonso, et al. (2002) to undertake a clinical trial of 1E10, a murine anti-idiotype mAb (Ab2) specific to P3 (Ab1), in 20 patients with advanced malignant melanoma. The 1E10 anti-idiotype vaccine was found to be safe, well tolerated, and immunologically effective, with some patients being able to generate a specific immune response against 1E10 (see FIG. 2 of Alfonso, et al. 2002) and NeuGcGM3 (see FIG. 4 of Alfonso, et al. 2002).

P3 was subsequently chimerized with a human IgG1 Fc region to form the chimeric antibody, chP3 (López-Requena, et al. 2003); and a single amino acid mutation (positively charged arginine substituted for negatively charged glutamic acid in position 99 of the V_{H} chain) yielded antibody chP3R99 (Fernández-Marrero, et al. 2011). The latter demonstrated: 1) increased reactivity with isolated NeuGcGM3 in ELISA, 2) increased reactivity with isolated sulfated GAGs in ELISA, and 3) complement-independent cytotoxicity for the NeuGcGM3-positive murine lymphocytic leukemia line, L1210, previously unseen for the wild type P3.

Cytotoxicity was determined by FCM of propidium iodide-stained L1210 cells incubated with chP3R99 in 1% FCS at 37° C. for 3 h. According to the recommendations of the American Type Culture Collection (ATCC), however, L1210 cells should be maintained in Dulbecco's Modified Eagle's Medium (DMEM) supplemented with 10% horse serum. Thus, growing L1210 cells in 1% FCS would be a form of serum starvation. Under these somewhat harsh conditions, Fernández-Marrero, et al (2011) observed that chP3R99, but not chP3, exerted a cytotoxic effect on L1210 cells in vitro (see FIG. 2B of Fernández-Marrero, et al 2011). Aside from the NeuGcGM3-positive murine cell line, L1210, the prior art discloses no other examples of cancer cells, either murine or human, being tested or treated with chP3R99.

Example 5 of U.S. Pat. No. 8,470,322 discloses that BALB/c mice immunized with chP3R99 developed anti-Hep antibodies via a vaccine effect, however, Fernández-Marrero, et al (2011) opined that said anti-Hep antibodies do not play any role in the chP3R99 mechanism of cytotoxicity. The authors state: “Our present results with the more reactive P3 E₉₉→R mutant suggest that affinity is crucial for the induction of this cytotoxic mechanism through targeting of GM3(Neu5Gc) [emphasis added].”

The present invention discloses antibodies (i.e., ICLX-1, 1HS, 3HS, and AO4B08) that, like chP3R99, exhibit polyspecific anti-GAG reactivity and are cytotoxic to cancer cells in vitro, but unlike chP3R99, do not exhibit any reactivity with NeuGcGM3.

The heavy and light chain CDR sequences (Kabat) of antibody P3, with their corresponding CW motifs are represented in the Table below. Histidine residues are indicated as B because histidine behaves as a basic amino acid as the pH nears 6.0.

| P3 | CDR1 | CDR2 | CDR3 |
|---|---|---|---|
| VH | RYSVH | MIWGGGSTDYNSALKS | SGVREGRAQAWFAY |
| | B--XB | XX-XXX-XO---XXB- | -XXBOXBX-X-XX- |
| VL | KASQDVSTAVA | SASYRYT | QQHYSTPWT |
| | BX--OX-XXXX | -X--B-X | --B--X--X |

The substitution of positively charged arginine for negatively charged glutamic acid in the VHCDR3 (Kabat) of antibody P3 represented a rational improvement in the quality of the CW motif in a critically important region of an antibody molecule. The effect on the overall antibody-GAG interaction was predictably favorable since VHCDR3 accounts for ~50% of the affinity of an antibody for its antigen.

Although P3 was initially developed as an antibody to NeuGcGM3, Soto, et al. (2012) noted that chP3 (an antibody with identical specificity as P3) also binds to sulfated GAGs in ELISA (see FIG. 1 of Soto, et al. 2012). In hindsight, one might speculate that the patients described in Alfonse, et al. (2002) who received the 1E10 anti-idiotype vaccine (Ab2) and who developed anti-anti-idiotype antibodies (Ab3) that cross-reacted with NeuGcGM3 (see FIG. 4 of Alfondo, et al. 2002) also could have developed antibodies that cross-reacted with sulfated GAGs, since chP3 cross-reacts with both NeuGcGM3 and sulfated GAGs. But the presence of antibodies to sulfated GAGs was never proven serologically in patients who received the 1E10 anti-idiotype vaccine (theoretically, these could have been anti-Hep, anti-HS, anti-CS, and anti-DS antibodies; see FIG. 1 of Soto, et al. 2012).

In addition to its use in patients with advanced melanoma, antibody 1E10 has undergone clinical trials in patients with non-small cell lung cancer (Alfonso, et al. 2007) and metastatic breast cancer (Soriano, et al., 2011). A humanized version of 1E10 is disclosed in U.S. Pat. No. 8,758,753.

Most recently, antibody chP3R99 has been used in pre-clinical radioimaging (Soto, et al. 2014) and therapeutic studies (Brito, et al. 2017) of atherosclerosis. There are no reports of the use of antibodies P3, chP3, or chP3R99 in any tumor xenograft models, or of their use as anti-cancer vaccines.

### Human Antibodies to Heparin and Heparan Sulfate Generated by Phage Display

van Kuppevelt, et al. (1998) used phage display technology to generate 3 human scFvs (HS4C3, HS4D10, and HS3G8) that were screened for reactivity against HS from bovine kidney. All 3 cross-reacted with Hep, but not with other GAGs including CSA, CSC, DS, KS, and HA. K_{d} values for HS were ~0.1 μM as determined by indirect competition ELISA. In addition to staining rat kidney sections by IHC, the 3 scFvs blocked bFGF binding to HS. This was the first report of the use of phage display technology for the generation of antibodies to polysaccharides. There are no reports of the use of antibodies HS4C3, HS4D10, and HS3G8 in any tumor xenograft models.

van de Westerlo, et al. (2002) used phage display technology to select 19 unique human anti-Hep antibodies. Some reacted almost exclusively with Hep, others cross-reacted with HS and CS. All 19 antibodies recognized a unique epitope. The effect of the antibodies on Hep as an anticoagulant was also studied. There were 3 antibodies that were very effective inhibitors of Hep action in the activated partial thromboplastin time (APTT) clotting assay, and their effect was related to the amount of Hep bound. Some antibodies reacted strongly with the pentasaccharide which interacts with antithrombin III.

Dennissen, et al. (2002) studied a panel of 10 unique anti-HS antibodies, which were generated using phage display technology. All 10 antibodies recognized a specific HS epitope in an ELISA against defined synthetic HS oligosaccharides, modified HS/Hep molecules, and HS isolated from a variety of organs. All 10 antibodies cross-reacted with Hep, but not other GAGs including DS, CS, KS, and HA. Due to their high degree of specificity and unique patterns of tissue reactivity, these antibodies most likely exemplify ‘lock and key’ protein-GAG interactions rather than the more common, electrostatic protein-GAG interaction. All 10 antibodies reacted with HS derived from bovine kidney. Six reacted with HS from human aorta. The latter included AO4B08, HS4E4, and EV3C3 (see Table II in Dennissen, et al. 2002). These same antibodies were subsequently tested by Christianson, et al. (2012) against U-87 MG glioma cells (vide infra).

Kurup, et al. (2007) characterized in detail two of the anti-HS antibodies described by Dennissen, et al. (2002). AO4B08 recognizes both HS and Hep, and was found to interact with a ubiquitous, N-, 2-O-, and 6-O-sulfated saccharide motif, including an internal 2-O-sulfate group. HS4E4, by contrast, HS4E4 recognizes low-sulfated HS motifs containing iduronic acid, and N-sulfated as well as N-acetylated glucosamine residues. Unlike AO4B08, HS4E4 did not bind to highly O-sulfated structures such as found in Hep.

The heavy and light chain CDR sequences (Kabat) of antibodies AO4B08 and HS4E4, with their corresponding CW motifs, are represented in the two Tables below. Histidine residues are indicated as B because histidine behaves as a basic amino acid as the pH nears 6.0. Aspartic acid and glutamic acid residues are marked with O because they are negatively charged and interact unfavorably with negatively charged GAGs. These sequences, as well as those of other anti-HS and anti-CS scFvs are disclosed in US2022/0227886.

| AO4B08 | CDR1 | CDR2 | CDR3 |
|---|---|---|---|
| VH | SYAMS | AISGSGGSTYYADSVKG | SLRMNGWRAHQ |
| | --XX- | XX-X-XX-X--XO-XBX | -XBX-X-BXB- |
| VL | QGDSLRSYYAS | GKNNRPS | NSRDSSGNHVV |
| | -XO-XB---X- | XB--B-- | --BO--X-BXX |

| HS4E4 | CDR1 | CDR2 | CDR3 |
|---|---|---|---|
| VH | NAWMS | RIKSKTDGGTTDYAAPVKG | HAPLRNTRTNT |
| | -X-X- | BXB-BXOXXXXO-XX-XBX | BX-XB-XBX-X |
| VL | QGDSLRSYYAS | GKNNRPS | NSRDSSGNHVV |
| | -XO-XB---X- | XB--B-- | --BO--X-BXX |

In a subsequent study that included anti-HS scFv antibodies AO4B08 and HS4E4 together with four other anti-HS scFv antibodies (HS3B7, HS3A8, EV3C3, and EW4G1), Thompson, et al. (2009) demonstrated that O-sulfates are not necessary for their binding; and that increasing sulfation does not necessarily correlate with increased antibody reactivity. Binding assays and IHC revealed that individual antibodies recognize distinct epitopes and that these are not single linear sequences but families of structurally similar motifs in which subtle variations in sulfation and conformation modify the affinity of interaction. Unexpectedly, there were significant differences between the existence of epitopes in tissue sections and observed in vitro dot blotted tissue extracts, demonstrating that in vitro specificity does not necessarily correlate with in vivo specificity.

Among five cell-surface-binding anti-HS scFv antibodies studied by Wittrup, et al. (2009), only antibody AO4B08 was found to efficiently translocate macromolecular cargo to intracellular vesicles through induction of HSPG endocytosis. Further analysis revealed the existence of both syndecan (i.e., a transmembrane HSPG) and glypican (i.e., a GPI-anchored HSPG) in purified vesicles. Importantly, internalized syndecan and glypican were found to co-localize in AO4B08-containing vesicles.

These results prompted Christianson, et al. (2012) to investigate the use of three human anti-HS scFv antibodies (AO4B08, HS4E4, and EV3C3; all originally described by Dennissen, et al. (2002)) as potential inhibitors of pro-angiogenic growth factor binding to HSPGs on the endothelial cells (ECs) of angiogenic tumors, in particular, glioblastoma. Unexpectedly, they found that all three anti-HS antibodies exhibited potent pro-angiogenic effects on primary human ECs. Mechanistically, the anti-HS antibodies triggered p38 MAPK-dependent signaling, resulting in increased proliferation of both ECs and glioblastoma cells. The latter was an unanticipated finding, which would argue strongly against the use of these 3 specific anti-HS scFv antibodies as anti-cancer agents. There are no reports of antibodies AO4B08, HS4E4, or EV3C3 in any tumor xenograft models.

The heavy and light chain CDR sequences (Kabat) of antibody EV3C3, with their corresponding CW motifs, are represented in the Table below. Histidine residues are indicated as B because histidine behaves as a basic amino acid as the pH nears 6.0. Aspartic acid and glutamic acid residues are marked with O because they are negatively charged and interact unfavorably with negatively charged GAGs. These sequences, as well as those of other anti-HS and anti-CS scFvs are disclosed in US2022/0227886.

| EV3C3 | CDR1 | CDR2 | CDR3 |
|---|---|---|---|
| VH | SNYMS | VIYSGGSTYYADSVKG | GYRPRF |
| | ---X- | XX--XX-X--XO-XBX | X-B-BX |
| VL | QGDSLRSYYAS | GKNNRPS | NSRDSSGNHVV |
| | -XO-XB---X- | XB--B-- | --BO--X-BXX |

U.S. Pat. No. 8,551,920 and Schoonbroodt, et al. (2008) disclose the development of an antibody repertoire enriched in antibodies that bind to small, charged carbohydrates and the construction of a human Fab phagemid library, “FAB-CCHO.” The latter combined L chain Ig sequences from human donors and H chain synthetic diversity constructed in key antigen contact sites in CDRs 1, 2 and 3 of the human framework V_{H}3-23. The H chain CDR3 was engineered to enrich the library in antibodies that bind charged carbohydrates by the introduction of basic residues at specific amino acid locations. These residues were selected on the basis of anti-carbohydrate antibody sequence alignment. The success of the design was demonstrated by the isolation of phage antibodies against charged carbohydrate therapeutic target antigens, including HS. Three anti-HS antibodies, referred to as 1HS, 2HS, and 3HS in U.S. Pat. No. 8,551,920, are described.

The heavy and light chain CDR sequences (Kabat) of antibody 1HS, with their corresponding CW motifs, are represented in the Table below. Histidine residues are indicated as B because histidine behaves as a basic amino acid as the pH nears 6.0. Aspartic acid and glutamic acid residues are marked with O because they are negatively charged and interact unfavorably with negatively charged GAGs. These sequences are disclosed in U.S. Pat. No. 8,551,920.

| 1HS | CDR1 | CDR2 | CDR3 |
|---|---|---|---|
| VH | NYTMA | SISSSGGHTPYADSVKG | GKRNRN |
| | --XXX | -X---XXBX--XO-XBX | XBB-B- |
| VL | KSSQSVLYSSNNKNYLA | WASTRES | QQYYSTPPT |
| | B----XX-----B--XX | -X-XBO- | -----X--X |

The VHCDR3 (Kabat) of antibody 1HS is comprised of an almost ideal CW motif (XBB-B- instead of XBBXBX) and could be easily improved by someone skilled in the art through the substitution of two hydropathic amino acid (e.g., glycine or alanine) for the two asparagines.

For antibody 1HS, the framework region between VHCDR2 (Kabat) and VHCDR3 (Kabat) is of particular interest because the final amino acids thereof, when taken together with VHCDR3 (Kabat), form an almost ideal extended CW motif: ARGKRNRN (=XBXBB-B-).

Delcommenne and Klingemann (2012) used a phage display library to generate human scFvs that reacted specifically against multiple myeloma (MM) cells. From 11 scFvs that were isolated, two (D4A4 and D6B10) stained MM cell lines and patient MM cells with higher intensity than normal plasma cells. Both scFvs immunoprecipitated syndecan-1 from MM cells and recognized sulfated motifs on syndecan-1-associated HS chains (syndecan-1 is the major marker of normal and transformed plasma cells; see Wijdenes, et al., 1996). Antibody D6B10 recognized HS 2,6-O-, N-sulfated motifs. In contrast, antibody D4A4 required N-sulfation combined with either 2-O- or 6-O-sulfation. Chondroitinase ABC did not block staining of MM cells by either antibody, but Hep, even at very low concentrations, completely blocked cell staining. A succession of experiments led to the conclusion that scFvs D4A4 and D6B10 recognize and bind to two different, but overlapping, syndecan-1 associated HS motifs that are overexpressed on MM cells while poorly expressed (D4A4) or absent (D6B10) from normal plasma cells.

U.S. Pat. No. 9,206,257 B2 and Gao, et al. (2014) describe human antibodies that bind with high affinity to glypican-3 (GPC3), or HS associated with GPC3. GPC3 is highly expressed in 70-100% of hepatocellular carcinomas (HCCs) but not in normal adult tissues. Initially, the antibodies were produced as scFvs by phage display, then converted to IgGs for tumor xenograft studies. One antibody, HS20, which recognizes HS chains on GPC3, inhibited Wnt3a-dependent cell proliferation in vitro and HCC xenograft tumors in BALB/c nu/nu mice without detectable in vivo toxicity. Antibody HS20 failed to bind to any of the glycan arrays of the Consortium for Functional Genomics, indicating that it does not bind to isolated HS, only HS in association with GPC3. A subsequent study by Gao, et al (2015), indicated that HS20-treated HCC cells exhibited less ability for hepatocyte growth factor (HGF)-mediated migration and motility. The antibodies of the present invention represent an improvement over HS20 because the former are not restricted to HS associated with only one type of HSPG, whereas the latter only binds to HS side chains that are directly bound to a single type of HSPG (i.e., glypican-3).

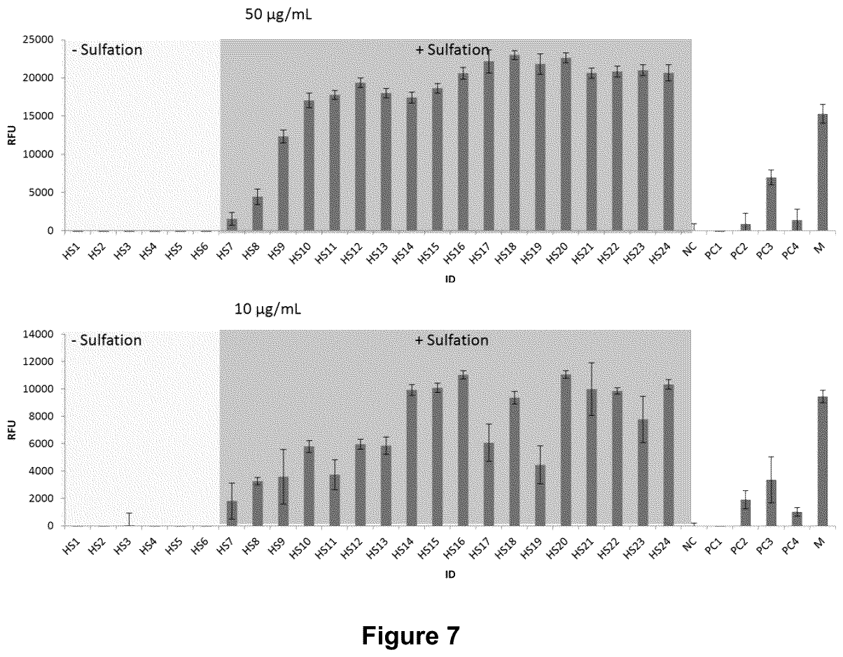

WO 2018/235855 A1, JP 7276855 B2 and US 2020/0283510 A1, and Katoh, et al. (2017) disclose immunogenetic repertoire profiling of tumor-infiltrating B- and T-cells in gastric cancers. Sulfated GAGs were identified as the major functional B-cell antigens. Two anti-sulfated GAG antibodies (#012T-1 and #013T-2 in Katoh, et al. 2017), were constructed from dominantly expressed heavy and light chain IgG genes. The former exhibited growth-suppressive functions in vitro against a variety of tumors, using an MTT assay. Both antibodies bound primarily to Hep when tested against a glycan microarray (see FIG. 7S of Katoh, et al., 2017), and were able to suppress the growth of at least one pancreatic and one lung cancer cell line in an MTT assay. In addition, antibody #013T-2 exhibited growth-suppressive effects on colorectal carcinoma and HCC cell lines. Neither #012T-1 nor #013T-2 were derived from individual B-cells.

This was the first report of an unconjugated antibody against an isolated sulfated GAG with a direct, antiproliferative effect on cancer cells in vitro, however, caution is required before drawing any conclusions about cell death based on the MTT assay. For example, the conversion of MTT to formazan depends on the metabolic activity of the cells, particularly mitochondrial activity. This means that the assay primarily measures metabolic activity rather than direct cell viability. Cells with altered metabolism, such as those under stress or undergoing differentiation, can give misleading results. More importantly, MTT itself is cytotoxic, which can inadvertently kill cells during the assay. This cytotoxicity can affect the accuracy of the assay, especially in prolonged incubations or with sensitive cell types.

The antibodies of the present invention differ from #012T-1 and #013T-2 in that the former cause necrotic cell death as determined by flow cytometry of propidium iodide-stained cells, whereas the latter were only shown to cause growth arrest in an MTT assay. Additionally, the antibodies of the present invention are polyspecific anti-GAG antibodies, whereas #012T-1 and #013T-2 react with only a single class of GAG (i.e., Hep/HS).

WO 2018/235855 A1, JP 7276855 B2 and US 2020/0283510 A1 disclose essentially the same results as Katoh, et al. (2017), except that the names of the antibodies have been changed from #012T-1 and #013T-2 to Gpat #2 and Gpat #1, respectively. Neither antibody was tested in a tumor xenograft model.

The heavy and light chain CDR sequences (Kabat) of antibody Gpat #1, with their corresponding CW motifs, are represented in the Table below. Histidine residues are indicated as B because histidine behaves as a basic amino acid as the pH nears 6.0. Aspartic acid and glutamic acid residues are marked with O because they are negatively charged and interact unfavorably with negatively charged GAGs.

| Gpat #1 | CDR1 | CDR2 | CDR3 |
|---|---|---|---|
| VH | DYAMH | GSSWDSGSIAYADSVKG | GLLPSLPYGMDV |
| | O-XXB | X---O-X-XX-XO-XBX | XXX--X--XXOX |
| VL | KSSQNIFYSSNNDNYLA | RASTWES | QQYYSTPYT |
| | B----XX-----O--XX | BX-X-O- | -----X--X |

Although some unconjugated anti-Hep antibodies may inhibit the proliferation of cancer cells in vitro (e.g., chP3R99 and Gpat #1), it is not obvious that they would have utility in an intact mammalian organism due to concerns about a potential anticoagulant effect (see van de Westerlo, et al. 2002).

WO 2023/086998 discloses a rare heparin [sic] sulfate octasaccharide (rHS) with the formula GlcNS6S-GlcA-GlcNS6S-IdoA2S-GlcNS6S-IdoA2S-GlcNS6S-GlcA, that is the preferred ligand for DC-HIL (also known as gpnmb). The rHS octasaccharide is identical to the octasaccharide “HSO21” used in the HS microarrays of the present invention. The antibodies of the present invention therefore cross-react with the rHS octasaccharide disclosed in WO 2023/086998.

WO 2023/086998 also discloses 2 single domain antibodies (sdAbs), based on a human V_{H} chain, that bind to rHS (HSO21). These are called 1A7 and 1F6, respectively. Apparently, “ . . . targeting the [rHS] glycan with a single domain antibody may be sufficient to prevent or reduce cancer progression and metastasis.” Human sdAbs (also called nanobodies) are not whole antibodies or antibody fragments in the strictest sense of the term, but rather antibody heavy or light chains. Human antibodies are normally comprised of at least 2 identical heavy and 2 identical light chains. Only camelids and certain cartilaginous fishes produce antibodies that are comprised of 2 identical heavy chains.

In Example 11 of WO 2023/086998, B57BL/6 mice were implanted s.c. with 2×10⁵ B16 (melanoma) or LL2 (lung carcinoma) cells, and starting on Day 6 post-implantation, injected with sdAb 1A7 every 2 days×5. The dose administered was 10 mg i.p. per mouse, which seems impossibly high. One mouse typically weighs 0.02 kg, therefore a dose of 10 mg/mouse translates to 500 mg/kg. For a typical 70 kg human, this would equate to 35 gm of sdAb, which seems rather ambitious. Additionally, human antibodies derived from cell culture are often supplied at a final concentration of ~0.5 mg/mL in PBS. Thus, administering a dose of 10 mg i.p. to a mouse would necessitate injecting >10 mL of fluid into its peritoneal cavity. Again, a rather impossible feat, especially if it had to be repeated every other day for a total of 5 injections.

WO 2023/086998 is therefore not sufficiently enabling insofar as the mouse experiments are concerned. Nor does WO 2023/086998 disclose whether sdAbs 1A7 or 1F6 cross-reacts with Hep or any other class of sulfated GAG, such as CS, DS, or KS. Given that other antibodies to “rare saccharide epitopes”, such as the 4 antibodies described by van den Born, et al. (2005) do not cross-react with Hep, it is not obvious that sdAbs 1A7 or 1F6 would also cross-react with Hep.

Additionally, sdAbs 1A7 and 1F6 represent individual Ig V_{H} chains, and thus not whole antibody variable regions, the later comprising both a V_{H} and a V_{L} chain. WO2023/086998 does not teach how to combine sdAbs 1A7 or 1F6 with an Ig V_{L} chain to form a whole antibody, Fab, or scFv that binds to rHS; nor is it clear that such a feat is even possible.

The CDR sequences (Kabat) of sdAb 1A7 and sdAb 1F6, with their corresponding CW motifs, are represented in the Tables below. Histidine residues are indicated as B because histidine behaves as a basic amino acid as the pH nears 6.0. Aspartic acid and glutamic acid residues are marked with O because they are negatively charged and interact unfavorably with negatively charged GAGs.

| 1A7 | CDR1 | CDR2 | CDR3 |
|---|---|---|---|
| VH | TKSMG | TIARNDGSTYYADSVKG | MKKNGGSKRQPHIRS |
| | XB-XX | XXXB-OX-X--XO-XBX | XBB-XX-BB--BXB- |

| 1F6 | CDR1 | CDR2 | CDR3 |
|---|---|---|---|
| VH | SQAMG | AIRKGNGSTYYADSVKG | KAYIKSQLGS |
| | --XXX | XXBBX-X-X--XO-XBX | BX-XB--XX- |

In retrospect, NovoMAb-G2 scFv was the first directly administered, unconjugated anti-GAG antibody to demonstrate an anti-cancer effect in vivo. It was well tolerated in both animal and human studies and did not cause any coagulopathies at therapeutic doses. NovoMAb-G2 scFv's specificity for GAGs was not known at the time, and it wasn't until several years later that the NovoMAb-G2 antigen was presumed to be CSA. However, as disclosed herein, NovoMAb-G2 scFv does not bind to CSA. It binds primarily to Hep with 10 to 30 degrees of polymerization (dp), less well to CSD with dp20, and very weakly to various forms of HS.

### Review of the NovoMAb-G2 (H11) Prior Art

### a) Method of Production of NovoMAb-G2 (H11)

WO97/44461, U.S. Pat. Nos. 6,207,153, 7,115,722, 7,166,286, and CA2255540 disclose a human antibody, H11, with alleged pan-carcinoma specificity and very limited binding to normal tissues. The H11 antigen is not disclosed and is simply referred to as C-antigen. The latter is “found specifically on neoplastic cells and not normal cells”.

The original H11 antibody (also called NovoMAb-G2, or VB1-011 in US2009/0075878 and U.S. Pat. No. 9,109,223) was an IgMK. It was produced and secreted by the human-human hybridoma NBGM1/H11 (Maiti, et al. 1997). The latter was prepared by fusing peripheral blood mononuclear cells (PBMCs) from a 63-year-old male with a grade II astrocytoma with the proprietary human myeloma-like cell line, TM-H2-SP2, and screening for reactivity to tumor cell lines.

The method of human-human hybridoma production and antibody screening used to develop antibody H11 (NovoMAb-G2) is described in Dan M (1989) and Dan M, et al. (1992). Anyone skilled in the art ought to be able to adapt this method to the task of finding novel human antibodies to Hep by fusing PBMCs from cancer patients with a suitable human fusion partner and screening the hybridoma supernatants either against ELISA plates coated with Hep, or against printed glycan microarrays similar to the ones available from the Consortium for Functional Glycomics (www.functionalglycomics.org).

Cancer patient PBMCs may not be the only source of B-lymphocytes that produce human antibodies to Hep. Stuchliková, et al. (1970) noted the presence of naturally occurring anti-Hep antibodies in otherwise healthy individuals, as well as an increase in their relative frequency with age. More recently, György, et al (2008) disclosed the presence of natural autoantibodies reactive with GAGs in adult controls and patients with rheumatoid arthritis. Much like the antibodies of the present invention, the anti-GAG antibodies described by György, et al (2008) showed significant cross-reactivity among different classes of GAG.

b) Ambiguities Surrounding the Sequence and Construction of H11 scFv

WO97/44461, U.S. Pat. Nos. 6,207,153, 7,115,722, 7,166,286, and CA2255540, describe the cloning and sequencing of immunoglobulin heavy and light chains from the original H11 IgMK, and the use thereof to construct an H11 scFv. Single-chain Fvs consist of a V_{H} chain linked to a V_{L} chain, either as V_{H}-linker-V_{L}-polyHis tail, or as V_{L}-linker-V_{H}-polyHis tail. Typically, the linker has the formula (G₄S)₃, but sometimes a shorter linker is preferred. The order of the variable chains (V_{H}→V_{L} or V_{L}→V_{H}) rarely has any impact on binding (although it may affect production levels in a given system); however, the shorter the linker the greater the chances of the scFv forming dimers, trimers, or other multimeric structures.

WO97/44461, U.S. Pat. Nos. 6,207,153, 7,115,722, 7,166,286, and CA2255540 disclose two equally preferable designs for the H11 scFv: SEQ ID NOs:14 and 17, with the former allegedly forming scFv monomers and the latter allegedly forming scFv dimers. The difference between the two is in the length of the linker joining the two variable regions. The H11 VHCDR3 (Kabat) hypervariable region, according to both SEQ ID NOs:14 and 17, is DQSLLGDYDHYYGLDV (Kabat numbering system).

WO97/44461, U.S. Pat. Nos. 6,207,153, 7,115,722, 7,166,286, and CA2255540 also disclose SEQ ID NO:2, which corresponds to the isolated V_{H} chain of H11. The H11 VHCDR3 (Kabat) hypervariable region, according to SEQ ID NO:2, is QSLLGDYDHYYGLDA

(Kabat numbering system). Thus, according to SEQ ID NO:2 and SEQ ID NOS:14 of WO97/44461, antibody H11 has two different VHCDR3 (Kabat) hypervariable region sequences, which is of course impossible.

The differences between these two VHCDR3 (Kabat) hypervariable region sequences are not insignificant. Aspartic acid is a negatively charged amino acid, and the VHCDR3 (Kabat) of SEQ ID NO:14 of WO97/44461 contains 4 such residues, as opposed to the VHCDR3 (Kabat) of SEQ ID NO:2, which contains only 3 aspartic acids. On the basis of electrostatic charge alone, one would expect weaker binding of SEQ ID NO:14 to a negatively charged target, such as a sulfated GAG, compared with SEQ ID NO:2. Even the terminal valine of the VHCDR3 (Kabat) of SEQ ID NO:14 would be expected to have an unfavorable effect on binding compared to the terminal alanine of the VHCDR3 (Kabat) of SEQ ID NO:2 because valine has three hydrophobic atoms compared to alanine's one (Al Mughram, et al. 2023).

WO97/44461, U.S. Pat. Nos. 6,207,153, 7,115,722, 7,166,286, and CA2255540 state: “This invention encompasses compositions containing antigen binding fragments of an antibody where the antibody specifically recognizes the antigen recognized by an antibody comprising a H chain V region having the amino acid sequence of SEQ ID NO:2 and a L chain V region having the amino acid sequence of SEQ ID NO:4.” From this statement it may be concluded that the V_{H} region of H11 is represented by SEQ ID NO:2. On the other hand, the major claim of the above patents is: “A composition comprising an antigen binding polypeptide fragment which specifically recognizes an epitope on C-antigen recognized by an scFv comprising SEQ ID NO:14.” From this statement it may be concluded that the V_{H} region of H11 is represented by SEQ ID NO:14. But SEQ ID NO:2 and SEQ ID NO:14 of WO97/44461 have different VHCDR3 (Kabat) hypervariable regions.

US2004/0091484 claims an antigen binding fragment of an antibody with a V_{H} region based on SEQ ID NO:2 and a V_{L} region based on SEQ ID NO:5 of WO97/44461, but offers no proof that such an antibody was ever produced or tested.

There is also ambiguity surrounding which H11 scFv constructs are the most preferable. WO97/44461, U.S. Pat. Nos. 6,207,153, 7,115,722, 7,166,286, and CA2255540 state that “The scFvs can be assembled in any order, for example, V_{H}-(linker)-V_{L} or V_{L}-(linker)-V_{H}. For example, SEQ ID NOs:13 and 16 show H11 scFv constructs having the form V_{L}-(linker)-V_{H}. However, the construct shown in SEQ ID NO:13 forms monomers, while the construct shown in SEQ ID NO:16 forms dimers. There may be differences in the level of expression of these two configurations in particular expression systems, in which case one of these forms may be preferred. Tandem scFvs can also be made, such as (X)-(linker)-(X)-(linker)-(X), in which X are αC polypeptides, or combinations of αC polypeptides with other peptides. In another embodiment, single chain antibody polypeptides have no linker peptide, or just a short, inflexible linker. Exemplary configurations include V_{L}-V_{H} and V_{H}-V_{L}. The linkage is too short to permit interaction between V_{L} and VH within the chain, and the chain forms homodimers with a V_{L}NH antigen binding site at each end. Such molecules are referred to in the art as “diabodies”.”

WO97/44461, U.S. Pat. Nos. 6,207,153, 7,115,722, 7,166,286, and CA2255540 thus allow for ten “preferred” versions of the H11 scFv on the basis of whichever one is the most easily expressed in a particular system (e.g., bacterial, mammalian, etc.). There are two preferred versions of the H11 VHCDR3 (Kabat) hypervariable region, two preferred orders of the V_{H} and V_{L} chains, and two preferred linkers: a long linker that favors the formation of monomers, and a short linker that favors the formation dimers. Dimeric scFvs could theoretically be composed of two scFvs with the same V_{H}/V_{L} order (either both V_{H}→V_{L} or both V_{L}→V_{H}), but with different VHCDR3 (Kabat) hypervariable regions. The latter would represent bispecific H11 diabodies. Of the ten “preferred” H11 scFvs, two seem to be ‘most’ preferred: SEQ ID NOS:14 and 17; and only one is NovoMAb-G2 scFv.

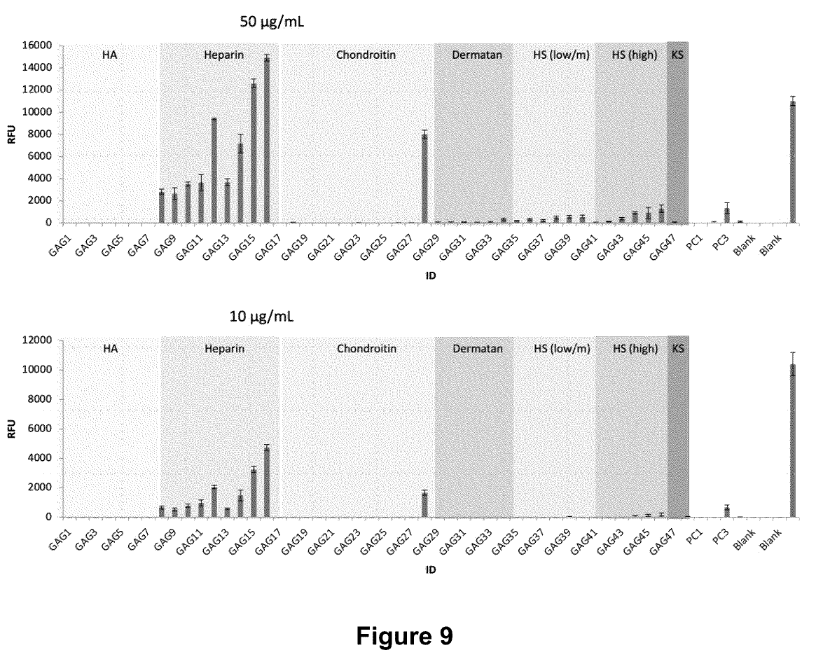

FIG. 9 of CA2288530 makes it clear that NovoMAb-G2 scFv is based on the VHCDR3 (Kabat) of SEQ ID NO:14 of WO97/44461 and not SEQ ID NO:2. And Ramjiawan, et al. (2000) and Reilly, et al. (2001) disclosed that NovoMAb-G2 scFv has a (G₄S)₃ linker, which would make it a monomer and not a dimer. They also disclosed that NovoMAb-G2 scFv may be utilized for imaging of A375 (melanoma) xenografts in SCID mice.

Perhaps the best way of making sense of this complicated situation is to understand that “H11” encompasses a range of antibodies and fragments of antibodies, including two possible VHCDR3 (Kabat) sequences; whereas “NovoMAb-G2 scFv” refers only to SEQ ID NO:14 of WO97/44461.

There is no disclosure in the prior art regarding the development and pre-clinical testing of an H11 scFv based on SEQ ID NO:2 of WO97/44461.

c) Pre-Clinical and Clinical Experience with NovoMAb-G2 IgM and NovoMAb-G2 scFv

A Phase I clinical trial with a radiolabeled version of NovoMAb-G2 IgM produced by the original NBGM1/H11 hybridoma was conducted at the University of Alabama at Birmingham (see Press Release, Jun. 17, 1998). Four patients with malignant melanoma were enrolled in the study. The results indicated that NovoMAb-G2 IgM was able to localize to known tumor sites. No significant adverse effects were noted. It's not known whether the NovoMAb-G2 IgM used in this trial had a heavy chain based on SEQ ID NO:2 or SEQ ID NO:14 of WO97/44461.

Subsequent pre-clinical studies demonstrated that unconjugated NovoMAb-G2 scFv produced in E. coli had an antiproliferative effect in the following tumor xenograft models: Daudi (Burkitt's lymphoma), GI-101 (breast carcinoma), A375 and GI-105 (melanoma), and PC-3 (prostate carcinoma) (see Press Releases, May 26, 1999, Oct. 7, 1999, and Apr. 4, 2000).

A Phase I clinical trial with NovoMAb-G2 scFv was conducted at the British Columbia Cancer Agency in Vancouver, British Columbia. Patients with progressive B-cell non-Hodgkin's lymphoma were given radiolabeled NovoMAb-G2 scFv, which demonstrated localization to known tumor sites (see Percheson, et al. (1999); Press Release, Mar. 25, 1999). As in the previous Phase I study with NovoMAb-G2 IgM, no significant adverse effects were reported. Unlike the previous Phase I trial, however, it's clear that these patients received a version of NovoMAb-G2 with a heavy chain based on SEQ ID NO:14 of WO97/44461.

A follow-on Phase I/II clinical trial involving unconjugated NovoMAb-G2 scFv demonstrated a positive response in 4 of 12 patients with end-stage cancer. One patient with stage IV breast cancer received 100 doses of NovoMAb-G2 scFv without any major incident, demonstrating a durable partial response manifested through dramatic reduction of multiple metastatic skin lesions (see Press Release of Jul. 23, 2002, and Laidman, 2002). As with the previous Phase I study, the version of NovoMAb-G2 that these patients received had a heavy chain based on SEQ ID NO:14 of WO97/44461.

### d) Ambiguities Surrounding the Antigen Recognized by NovoMAb-G2

Previous attempts at characterizing the antigen recognized by the hybridoma-secreted H11 IgM were unsuccessful. After NovoMAb-G2 scFv was developed, attempts at characterizing the antigen recognized by NovoMAb-G2 scFv were met with only limited success. WO97/44461, U.S. Pat. Nos. 6,207,153, 7,115,722, 7,166,286, and CA2255540 describe the H11 antigen in terms of SEQ ID NO:14's ability to react with a series of heptapeptide consensus sequences.

Subsequently, CA2290722A1 described the NovoMAb-G2 scFv antigen as a complex of an unidentified peptide associated with a heat shock protein (Hsp70 and 90).

Still later, WO2006/105653, U.S. Pat. Nos. 7,956,162, 8,389,286, and 8,946,390 claim that the NovoMAb-G2 scFv antigen is a variant of mammalian scratch protein “in association with” chondroitin sulfate A (CSA), and to a lesser extent HA. The CSA component was never proven, but rather, inferred because “Extensive experimentation revealed that the glycan modification involved a soluble form of CS (chondroitin sulfate); two of these (CSB and CSE) have limited tissue distribution. As such, the glycan modification could be attributable to CSA and to a lesser extent hyaluronic acid”.

And finally, US2009/0075878 and U.S. Pat. No. 9,109,223 state, in a roundabout way, that the NovoMAb-G2 scFv antigen is simply CSA, without any reference to the mammalian scratch protein variant disclosed in WO2006/105653, U.S. Pat. Nos. 7,956,162, 8,389,286, and 8,946,390.

Example 4 of US2009/0075878 and U.S. Pat. No. 9,109,223 compares the binding of an affinity-matured version of VB6-011 (so called VB6-011 2D3) against the “wild type” VB6-011 in an “ELISA against immobilized chondriotin [sic]sulfate”. VB6-011 is described as an immunoconjugate of the Fab portion of VB3-011 linked to a de-immunized version of the ribosome inactivating protein bouganin (so called ‘de-bouganin’), as disclosed in U.S. Pat. Nos. 7,956,162, 8,389,286, 8,946,390, 9,109,223, and EP1737961. VB3-011 is the IgG version of VB1-011, “an antibody with the variable region of the antibody disclosed in WO97/44461 which has been shown to specifically bind to a variety of cancer cells and does not specifically bind to normal tissue or cells.” Thus, the antigen recognized by VB6-011 is the same as the antigen recognized by VB6-011-2D3, VB3-011, VB2-011, and VB1-011. VB2-011 is another name for NovoMAb-G2 scFv (see Press Release, Jul. 23, 2002), which is a monomeric H11 scFv based on SEQ ID NO:14 of WO97/44461. As for the CS that was used in the above ELISA, its source is not disclosed and it may well have included a mixture of chondroitin sulfates, including CSD.

The heavy and light chain CDR sequences (Kabat) of NovoMAb-G2 scFv (VB2-011) and VB6-011 2D3, with their corresponding CW motifs, are represented in the Tables below. Histidine residues are indicated as B because histidine behaves as a basic amino acid as the pH nears 6.0. Aspartic acid and glutamic acid residues are marked with 0 because they are negatively charged and interact unfavorably with negatively charged GAGs. The affinity maturation process has resulted in a significant improvement in the quality of CW motifs, particularly in VLCDR1 (Kabat).

| VB2-011 | CDR1 | CDR2 | CDR3 |
|---|---|---|---|
| VH | SFAMH | VISYDGSTKYYADSVKG | DQSLLGDYDHYYGLDV |
| | -XXXB | XX--OX-XB--XO-XBX | O--XXXO-OB--XXOX |
| VL | RASQSVSSSYLA | GASTRAT | QQYGSSPOTPQIT |
| | BX---X----XX | XX-XBXX | ---X----X--XX |

| 011-2D3 | CDR1 | CDR2 | CDR3 |
|---|---|---|---|
| VH | AFAMH | VISYDGMCKSYADSVKG | DQTLLGDYDHYYMMDV |
| | XXXXB | XX--OXXXB--XO-XBX | O-XXXXO-OB--XXOX |
| VL | RALKAVWLEYLA | GASTRAT | LPCGGAPQTPQIT |
| | BXXBXX-XO-XX | XX-XBXX | X-XXXX--X--XX |

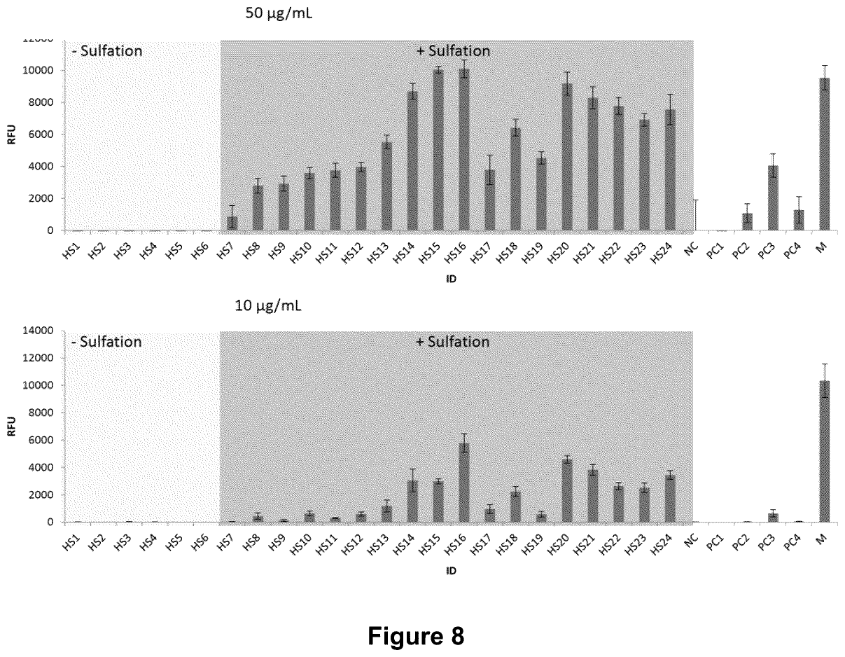

The caption for FIG. 8 of US2009/0075878 and U.S. Pat. No. 9,109,223 reads: “ELISA for CSA binding of affinity matured light chain VB6-011-ETA₍₂₅₂₋₆₀₈₎-VL2 compared with wild type and controls.” It is not clear, however, that CSA was specifically added to the ELISA microwells, as opposed to CS in general. The controls include VB6-011 (positive control), and 2 negative controls: 1) VB6-845, a recombinant fusion protein comprised of a humanized Fab anti-EpCAM antibody linked to de-bouganin (see MacDonald, et al. 2008), and 2) 3302, the E. coli vector with no insert. There is neither an anti-CS positive control, nor any statistical analysis of the ELISA results (or even standard error bars) in FIG. 8.

Thus, in the 12 years between the filing of WO97/44461 and US2009/0075878, the antigen recognized by NovoMAb-G2 scFv went from being a cancer-specific molecule that cross-reacted with a series of heptapeptide consensus sequences, to the common and ubiquitous glycosaminoglycan, CSA. The inherent utility of NovoMAb-G2 scFv, therefore, underwent a corresponding shift from an antibody that could potentially be used for the treatment of all forms of cancer, to one whose use is confined to cancers that only overexpress CSA, presumably in the ECM where it typically resides. This is problematic because CSA is a constituent of normal tissues and cells, and WO97/44461, U.S. Pat. Nos. 6,207,153, 7,115,722, 7,166,286, 7,956,162, 8,389,286, and 8,946,390 state explicitly that the NovoMAb-G2 scFv antigen is absent from normal tissues and cells.

Another important issue arising from the CSA hypothesis is that it fails to shed light on exactly how NovoMAb-G2 scFv, as an unconjugated antibody fragment with no effector function, can inhibit cancer growth in xenograft models and in human clinical trials. Clearly, it is not enough to conclude that NovoMAb-G2 scFv has anti-cancer properties. Which cancers it is effective against, and if possible, why, is one of the aims of the present disclosure.

Disclosed herein in the following examples are GAG and HS microarray results that demonstrate that NovoMAb-G2 scFv binds mainly to Hep, with a preference for longer chain lengths of Hep. It also binds to long-chain CSD, which is a high sulfation form of CS found predominantly in marine animals (e.g., whales and sharks). CSD plays only a minor role in human cancers (Vallen, et al. 2014; Pudełko, et al. 2019; Lv, et al 2007; Ucakturk, et al, 2016). At high concentrations, some weak binding of NovoMAb-G2 scFv to HS can be observed. But NovoMAb-G2 scFv binds neither to CSA nor HA, as suggested by the prior art.

Also disclosed herein in the following example are results demonstrating that NovoMAb-G2 scFv binding to HS increases at pH 6.8, which is more representative of the TME due to the Warburg effect. This may help to explain NovoMAb-G2 scFv's margin of safety: at physiological pH it interacts mainly with Hep (said interaction, as it turns out, being well tolerated by human subjects), but when it encounters a tumor cell, NovoMAb-G2 scFv binds to membrane-tethered HS due to the mildly acidic TME.

### Common Features of Anti-HS Antibodies and the Importance of Framework Regions

Human antibodies NovoMAb-G2 scFv, 1HS IgG1, Gpat #1, AO4B08, HS4E4, EV3C3, 1A7, and 1F6 were all developed independently using very different methods, yet they share the following features in common:
1) VHCDR1 (Kabat) has a methionine (CW motif=X) in the penultimate position
2) VHCDR2 (Kabat) ends with the amino acid sequence VKG (CW motif=XBX)

Additionally, antibodies NovoMAb-G2 scFv, 1HS IgG1, and Gpat #1 share the following features in their VLCDR1 (Kabat):
1) A basic amino acid (either arginine of lysine) in the initial position (CW motif=B)
2) The amino acid sequence LA (CW motif=XX) at the end

These commonalities should be viewed from the perspective of human IgG framework regions:
1) VHCDR2 (Kabat) is followed by the amino acids RF, thus a VHCDR2 (Kabat) that ends with the amino acid sequence VKG corresponds to the CW motif XBXBX
2) VLCDR1 (Kabat) is preceded by the amino acid cysteine, thus a basic amino acid in the initial position of VLCDR1 (Kabat) corresponds to the CW motif XB

Antibody chP3R99 is an example of an anti-GAG antibody with an ideal CW motif in its VHCDR3 (Kabat) region, but not all anti-GAG antibodies have this feature. More frequently, and as illustrated in this example, it was observed how framework regions blend into the CDR regions of anti-GAG antibodies to form CW motifs.

### Nomenclature of the Antibodies of the Invention

The present disclosure describes novel functions for an antibody (ICLX-3 scFv) which is functionally equivalent to NovoMAb-G2 scFv, and which was previously believed to bind only to CSA. Also described are 4 other antibodies closely related to NovoMAb-G2 scFv. For the sake of clarity and simplicity, these 5 antibodies are named and described as follows:
a. ICLX-1 is based on SEQ ID NO:2 and SEQ ID NO:4 of WO97/44461. The VHCDR3 (Kabat) of ICLX-1 is: QSLLGDYDHYYGLDA (SEQ ID NO: 1). This antibody represents one of the ten “preferred” versions of the H11 scFv. It has not been studied before.
b. ICLX-2 is closely related to ICLX-1, differing only by the substitution of valine for alanine in the terminal position of VHCDR3 (Kabat). The VHCDR3 (Kabat) of ICLX-2 is: QSLLGDYDHYYGLDV (SEQ ID NO: 2). This antibody has neither been previously described nor studied.
c. ICLX-3 is based on SEQ ID NO:14 of WO97/44461. This antibody is functionally equivalent to NovoMAb-G2 scFv. The VHCDR3 (Kabat) of both ICLX-3 and NovoMAb-G2 scFv is: DQSLLGDYDHYYGLDV (SEQ ID NO: 3). NovoMAb-G2 scFv has been previously studied in pre-clinical, Phase I, and Phase I/II trials, however, NovoMAb-G2 scFv was constructed with a V_{L}→V_{H} orientation and contains additional amino acid sequences after the V_{L}. These additional sequences are not included in the ICLX-3 scFv of the present invention. NovoMAb-G2 scFv is therefore not identical to ICLX-3 scFv, even though they share identical Kabat CDR regions.
d. ICLX-4 is based on a modification of SEQ ID NO:2, and an unmodified SEQ ID NO:4, of WO97/44461. The VHCDR3 (Kabat) of ICLX-4 is: QSLLGDYDRYYGLDA (SEQ ID NO: 4).
e. ICLX-5 is based on a modification of SEQ ID NO:2, and an unmodified SEQ ID NO:4, of WO97/44461. The VHCDR3 (Kabat) of ICLX-5 is: QSLLGDYDKYYGLDA (SEQ ID NO: 5).

Additionally, each antibody of the present invention is designated as either a scFv, IgG1, or IgG4 (e.g., ICLX-1 scFv, ICLX-1 IgG4, etc.).

Having thus introduced the field of endeavour of the present disclosure, the present invention will be more readily understood by describing specific embodiments.

According to a first aspect, the present invention is directed to ______ an antibody that binds to isolated sulfated glycosaminoglycans (GAGs), wherein the antibody comprises a Variable Heavy Chain Complementarity Determining Region 3 (VHCDR3) according to the Kabat numbering system having the amino acid sequence of SEQ ID NO: 6.

According to a preferred embodiment, the antibody is a single-chain variable fragment (scFv).

According to an embodiment, the antibody is a human scFv antibody.

According to an embodiment, said antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, the antibody comprises a VHCDR3 according to the Kabat numbering system having the amino acid sequence of any one of SEQ ID NO: 1-5.

According to an embodiment, the antibody described herein comprises a VHCDR3 according to the Kabat numbering system having the amino acid sequence of SEQ ID NO: 3.

According to an embodiment, the antibody comprises a VHCDR3 according to the Kabat numbering system having the amino acid sequence of SEQ ID NO: 1, 2, 4 or 5.

| | SEQ | VHCDR3 (Kabat) |
|---|---|---|
| Antibody | ID NO | Sequence |
| ICLX-1 | 1 | QSLLGDYDHYYGLDA |
| ICLX-2 | 2 | QSLLGDYDHYYGLDV |
| ICLX-3 | 3 | DQSLLGDYDHYYGLDV |
| ICLX-4 | 4 | QSLLGDYDRYYGLDA |
| ICLX-5 | 5 | QSLLGDYDKYYGLDA |
| ICLX-1 | 6 | X₁QSLLGDYDX₂YYGLDX₃ |
| ICLX-2 | | X₁, when present, is D; |
| ICLX-3 | | X₂ = H, R or K; and |
| ICLX-4 | | X₃ = A or V |
| ICLX-5 | | |

According to an embodiment, the antibody and heparin having a degree of polymerization of 24 (dp24) is less than 250 nM.

According to an embodiment, the K_{d} of the antibody and chondroitin sulfate D having a degree of polymerization of 20 (dp20) is less than 300 nM.

According to a second aspect, the present invention is directed to an antibody or fragment thereof with a VH or a VL region having at least 70%, at least 80%, or at least 90% homology to the VH or VL amino acid sequence of any one of SEQ ID NO: 7-11 and 21-23, wherein the antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to a preferred embodiment, the GAGs comprise isolated heparin, high sulfation heparan sulfate, low/intermediate sulfation heparan sulfate, and chondroitin-2,6-sulfate with 20 degrees of polymerization (dp20).

According to an embodiment, the antibody binds to isolated heparin, high sulfation heparan sulfate, low/intermediate sulfation heparan sulfate, and chondroitin-2,6-sulfate with 20 degrees of polymerization (dp20).

According to an embodiment, the K_{d} of the antibody and a heparan sulfate octamer with 2.5 sulfates/disaccharide (HS024) under mildly acidic conditions is less than 1 μM.

According to an embodiment, the mildly acidic conditions correspond to a pH of between 6.0 and 6.8.

According to an embodiment, the antibody comprises a methionine in the penultimate position of the VHCDR1 according to the Kabat numbering system.

According to an embodiment, the antibody comprises the amino acid sequence VKG at the C-terminal end of the VHCDR2 according to the Kabat numbering system.

According to an embodiment, the antibody comprises the amino acid sequence YLA at the C-terminal end of the VLCDR1 according to the Kabat numbering system.

According to an embodiment, the antibody comprises a VHCDR1 according to the Kabat numbering system of SEQ ID NO: 12.

According to an embodiment, the antibody comprises a VHCDR2 according to the Kabat numbering system of SEQ ID NO: 13.

According to an embodiment, the antibody comprises a VLCDR1 according to the Kabat numbering system of SEQ ID NO: 14.

According to an embodiment, the antibody comprises a VLCDR2 according to the Kabat numbering system of SEQ ID NO: 15.

According to an embodiment, the antibody comprises a VLCDR3 according to the Kabat numbering system of SEQ ID NO: 16.

According to a third aspect, the present invention is directed an antibody that binds to heparin, chondroitin-2,6-sulfate with 20 degrees of polymerization (dp20), and dermatan sulfate, wherein the antibody comprises the amino acid sequence of SEQ ID NO: 17, SEQ ID NO: 18, and/or SEQ ID NO: 22.

According to a fourth aspect, the present invention is directed to an antibody that binds to heparin, chondroitin-2,6-sulfate with 20 degrees of polymerization (dp20), and dermatan sulfate, wherein the antibody comprises an amino acid sequence of SEQ ID NO: 19, and/or SEQ ID NO: 20, and/or SEQ ID NO: 23.

According to a fifth aspect, the present invention is directed to anti-GAG antibody comprising a methionine in the penultimate position of the VHCDR1 according to the Kabat numbering system and that binds to any combination of 2 or more of the following 4 classes of glycosaminoglycan (GAG): heparin/heparan sulfate, chondroitin sulfate/dermatan sulfate, keratan sulfate, and hyaluronic acid

According to a sixth aspect, the present invention is directed to anti-GAG antibody comprising the amino acid sequence VKG at the C-terminal end of the VHCDR2 according to the Kabat numbering system and that binds to any combination of 2 or more of the following 4 classes of glycosaminoglycan (GAG): heparin/heparan sulfate, chondroitin sulfate/dermatan sulfate, keratan sulfate, and hyaluronic acid.

According to a seventh aspect, the present invention is directed to anti-GAG antibody comprising the amino acid sequence YLA at the C-terminal end of the VLCDR1 according to the Kabat numbering system and that binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, the anti-GAG antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an eighth aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid and that does not also bind to Neu5Gc-containing gangliosides.

According to a ninth aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid that does not also bind to Neu5Gc-containing gangliosides.

According to a tenth aspect, the present invention is directed to the antibody as described herein for use in killing cancer cells.

According to an embodiment, the antibody as described herein is for use in killing cancer cells ex vivo.

According to an eleventh aspect, the present invention is directed to the antibody as described herein for use in directly killing cancer cells.

According to a twelfth aspect, the present invention is directed to a pharmaceutical composition comprising the antibody as described herein and a pharmaceutically acceptable excipient or diluent.

According to a thirteenth aspect, the present invention is directed to a pharmaceutical composition comprising the antibody as described herein and a pharmaceutically acceptable excipient or diluent.

According to a fourteenth aspect, the present invention is directed to a pharmaceutical composition comprising an anti-GAG antibody and a pharmaceutically acceptable excipient or diluent.

According to a fifteenth aspect, the present invention is directed to a pharmaceutical composition comprising an anti-GAG antibody and a pharmaceutically acceptable excipient or diluent.

According to a sixteenth aspect, the present invention is directed to a method of treating cancer in a subject in need thereof. The method comprises administering a therapeutically effective amount of the antibody as described herein, or the pharmaceutical composition as described herein, to the subject.

According to an embodiment, the cancer aberrantly expresses a membrane-tethered heparan sulfate proteoglycan.

According to an embodiment, the membrane-tethered heparan sulfate proteoglycan is syndecan-1, syndecan-2, syndecan-3, syndecan-4, glypican-1, glypican-2, glypican-3, glypican-4, glypican-5, glypican-6, glypican-7, glypican-8, or any combination thereof.

According to an embodiment, the method further comprises administering a chemotherapeutic agent to the subject.

According to an embodiment, the method further comprises administering another anti-cancer agent to the subject.

According to a seventeenth aspect, the present invention is directed to the antibody or the pharmaceutical composition as described herein, for use in purging cancer cells from an autologous stem cell transplant (ASCT) or an autologous bone marrow transplant (ABMT).

According to an eighteenth aspect, the present invention is directed to the antibody or the pharmaceutical composition as described herein, for use in purging multiple myeloma, Hodgkin's lymphoma or neuroblastoma cells from an autologous stem cell transplant (ASCT) or autologous bone marrow transplant (ABMT).

According to a nineteenth aspect, the present invention is directed to the antibody or the pharmaceutical composition as described herein, for use in purging multiple myeloma, Hodgkin's lymphoma or neuroblastoma cells from an autologous stem cell transplant (ASCT) or autologous bone marrow transplant (ABMT).

According to a twentieth aspect, the present invention is directed to the antibody or the pharmaceutical composition as described herein, for use in autologous stem cell transplantation (ASCT) or autologous bone marrow transplantation (ABMT).

According to an embodiment, the antibody or pharmaceutical composition is for purging cancer cells from an autologous stem cell transplant (ASCT) or an autologous bone marrow transplant (ABMT).

According to an embodiment, the cancer cells are from multiple myeloma, Hodgkin's lymphoma or neuroblastoma.

According to a twenty first aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid for use in purging an autologous stem cell transplant (ASCT) or autologous bone marrow transplant (ABMT) of cancer cells that express a membrane-tethered heparan sulfate proteoglycan (HSPG).

According to an embodiment, the cancer cells are from multiple myeloma, Hodgkin's lymphoma, neuroblastoma or any other cancer in a subject that could benefit from an autologous stem cell transplantation (ASCT) or autologous bone marrow transplantation (ABMT).

According to a twenty second aspect, the present invention is directed to an anti-GAG antibody that binds to any combination of 2 or more of the following 4 classes of glycosaminoglycan (GAG): heparin/heparan sulfate, chondroitin sulfate/dermatan sulfate, keratan sulfate, and hyaluronic acid for use in purging an autologous stem cell transplant (ASCT) or autologous bone marrow transplant (ABMT) of hematopoietic stem cells (HSC).

According to a twenty third aspect, the present invention is directed to the antibody or the pharmaceutical composition as described herein, for use in purging an autologous stem cell transplant (ASCT) or autologous bone marrow transplant (ABMT) of hematopoietic stem cells (HSC).

According to a twenty fourth aspect, the present invention is directed to a method of killing cancer cells comprising exposing cancer cells to an effective amount of an anti-GAG antibody, wherein said antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, said antibody does not bind to Neu5Gc-containing gangliosides.

According to an embodiment, the anti-GAG antibody is polyspecific.

According to an embodiment, the method is for directly killing cancer cells.

According to a twenty fifth aspect, the present invention is directed to a method of performing an autologous stem cell transplantation (ASCT) or autologous bone marrow transplantation (ABMT), the method comprising: exposing the hematopoietic stem cells (HSC) of a subject to an anti-GAG antibody, wherein said antibody binds to any combination of 2 or more of the following 4 classes of glycosaminoglycan (GAG): heparin/heparan sulfate, chondroitin sulfate/dermatan sulfate, keratan sulfate, and hyaluronic acid.

According to an embodiment, the hematopoietic stem cells (HSC) are contaminated with, or presumed to be contaminated with, cancer cells.

According to an embodiment, the subject has been diagnosed with cancer.

According to an embodiment, the cancer is multiple myeloma, Hodgkin's lymphoma or neuroblastoma.

According to an embodiment, exposing the autologous hematopoietic stem cells (HSCs) to the anti-GAG antibody kills cancer cells, thereby purging the cancer cells from the autologous HSCs of the subject.

According to an embodiment, the anti-GAG antibody is the antibody as defined in any one of claims 1-26.

According to a twenty sixth aspect, the present invention is directed to the antibody or the pharmaceutical composition as described herein, for use in purging cancer cells and/or hematopoietic stem cells (HSC) from an autologous stem cell transplant (ASCT) or an autologous bone marrow transplant (ABMT).

According to an embodiment, the cancer cells are from multiple myeloma, Hodgkin's lymphoma, neuroblastoma or any other cancer in a subject that could benefit from an autologous stem cell transplantation (ASCT) or autologous bone marrow transplantation (ABMT).

The present invention will be more readily understood by referring to the following examples which are given to illustrate the invention rather than to limit its scope.

### EXAMPLES

### Example 1: Production of Different Versions and Variations of the H11 scFv

WO97/44461, U.S. Pat. Nos. 6,207,153, 7,115,722, 7,166,286, and CA2255540 suggest ten possible “preferred” versions of the H11 scFv based on: 1) the amino acid sequence of the VHCDR3 (Kabat), 2) the order of V_{H} and V_{L} domains within a given scFv, and 3) the preferred length of the linker segment.

Different versions and variations of the H11 scFv (Sino Biological and Bon Opus Biosciences) were generated, some of which failed to express in HEK293 cells, and some of which failed to refold properly in PBS pH 7.4.

Initial attempts at scFv production with V_{L} and V_{H} sequences in the same order as SEQ ID NO:14 of WO97/44461 (i.e., V_{L}→V_{H}) failed to express in HEK293 cells. It was only after the order was reversed to V_{H}→V_{L} that adequate expression levels of a functional antibody were achieve. Table 1 below summarizes key characteristics of three scFv antibodies termed ICLX-1, ICLX-2 and ICLX-3, synthesized by Sino Biological and Bon Opus Biosciences. Additional notes are provided below Table 1.

TABLE 1
Single-chain Fv synthesis

| scFv # | Company | order | sequence | linker | tail | Notes |
|---|---|---|---|---|---|---|
| 1 | Sino Biological | V_{L}→V_{H} | ICLX-2 | (G₃S)₃ | hFc | A |
| 2 | Bon Opus | V_{L}→V_{H} | ICLX-1 | (G₄S)₃ | poly-His | B |
| 3 | Bon Opus | V_{L}→V_{H} | ICLX-2 | (G₄S)₃ | poly-His | C |
| 4 | Bon Opus | V_{L}→V_{H} | ICLX-2 | G | poly-His | D |
| 5 | Sino Biological | V_{H}→V_{L} | ICLX-1 | (G₄S)₃ | poly-His | E |
| 6 | Sino Biological | V_{H}→V_{L} | ICLX-2 | (G₄S)₃ | poly-His | F |
| 7 | Sino Biological | V_{H}→V_{L} | ICLX-3 | (G₄S)₃ | poly-His | G |

A) Very low yield. Project abandoned.
B) The majority of the antibody precipitated in PBS, pH7.4. It had to be renatured in PBS with 0.5M L-arginine, etc. Tested poorly in GAG microarray due to solubilization agents.
C) Unable to renature in PBS pH7.4; only small amounts of intracellular protein could be renatured into PBS, 0.5M L-arginine, 5 mM GSH, 1 mM GSSG pH7.4 buffer.
D) Only part of the intracellular protein could be solubilized by 8M urea. Required addition of 0.5M L-arginine, etc. Did not form dimers.
E) No production issues. Compared against scFv #6 and #7 in GAG and HS microarrays.
F) No production issues. Compared against scFv #5 in GAG and HS microarrays.
G) No production issues. Compared against scFv #5 in GAG and HS microarrays.
H) Difficult to produce. Purity was only 45.5% by Coomassie Blue stain.
I) Difficult to produce. Initial purity was 97.7%, but the antibody degraded after just a few freeze-thaw cycles.

The orientation of V_{H} and V_{L} sequences within a given scFv is sometimes important for expression, secretion, and binding to the target antigen. Luo et al. (1995) described a scFv derived from the antibody MAb 174H.64 that could only be expressed and secreted in the recombinant Pichia pastoris system when constructed with a V_{L}→V_{H} orientation. The V_{H}→V_{L} version of the MAb 174H.64 scFv failed to express and secrete. In addition, Liu, et al. (2015) described two different versions of scFvs 2E6, both of which were expressed in E. coli as inclusion bodies. After refolding, only scFvs 2E6 with a V_{H}→V_{L} orientation retained bioactivity against its target antigen.

In present disclosure, poor scFv production in HEK293 cells was observed when the scFvs were constructed with a V_{L}→V_{H} orientation, and good production when they were constructed with a V_{H}→V_{L} orientation. Single-chain Fv sequence #4 was modelled on SEQ ID NO:17 of WO97/44461, with a very short linker sequence. Unfortunately, it failed to dimerize as expected.

### Example 2: Side-by-Side Comparison of ICLX-1 scFv and ICLX-2 scFv in GAG and HS Microarrays

ICLX-1 scFv has a heavy chain that is representative of SEQ ID NO:2 of WO97/44461. ICLX-2 scFv is closely related to ICLX-1 scFv, differing only by the substitution of valine for alanine in the final position of VHCDR3 (Kabat). The light chains of ICLX-1 scFv and ICLX-2 scFv are identical. The purpose of this experiment, therefore, was to evaluate the effect on anti-GAG binding affinity of this valine for alanine substitution in VHCDR3 (Kabat).

The heavy and light chain CDR sequences (Kabat) of ICLX-1 scFv and ICLX-2 scFv, with their corresponding CW motifs, are represented in Table 2. Histidine residues are indicated as B because histidine behaves as a basic amino acid as the pH nears 6.0. Aspartic acid and glutamic acid residues are marked with 0 because they are negatively charged and interact unfavorably with negatively charged GAGs.

TABLE 2
Heavy and light chain CDR sequences (Kabat) of ICLX-1 scFv and
ICLX-2 scFv, with their corresponding CW motifs

| ICLX-1 | CDR1 | CDR2 | CDR3 |
|---|---|---|---|
| VH | SFAMH | VISYDGSTKYYADSVKG | QSLLGDYDHYYGLDA |
| | -XXXB | XX--OX-XB--XO-XBX | --XXXO-OB--XXOX |
| VL | RASQSVSSSYLA | GASTRAT | QQYGSSPOTPQIT |
| | BX---X----XX | XX-XBXX | ---X----X--XX |
| ICLX-2 | CDR1 | CDR2 | CDR3 |
| VH | SFAMH | VISYDGSTKYYADSVKG | QSLLGDYDHYYGLDV |
| | -XXXB | XX--OX-XB--XO-XBX | --XXXO-OB--XXOX |
| VL | RASQSVSSSYLA | GASTRAT | QQYGSSPOTPQIT |
| | BX---X----XX | XX-XBXX | ---X----X--XX |

Since both valine and alanine are hydropathic amino acids, the substitution of one for the other was not expected to significantly affect the overall CW motifs of the VHCDR3 (Kabat). However, valine has three hydrophobic atoms compared to alanine's one (Al Mughram, et al. 2023), and this would be expected to impact the quality of the CW motif.

### Method

scFv #5 (ICLX-1 scFv) and scFv #6 (ICLX-2 scFv), both described in Table 1 were prepared by Sino Biological and tested by Z Biotech, LLC on the same standard GAG microarray and on the same day. The microarray was pretreated with Glycan Array Blocking Buffer (GABB) at room temperature for one hour. After blocking, a pre-complexed mixture of ICLX-1 scFv or ICLX-2 scFv, anti-his tag mAb, and anti-mouse IgG (H+L) AF555 was added to the microarray and incubated at room temperature for 1 hour. The precomplexed mixture initially contained ICLX-1 scFv or ICLX-2 scFv at at 10 μg/mL or 50 μg/mL concentration, anti-his tag mAb at 1:500 dilution, and anti-mouse IgG (H+L) AF555 at 2 μg/mL concentration. This mixture was then diluted by factors of 5 to 6 times. After incubation at room temperature for 1 hour, the microarray was washed and scanned at 532 nm using high laser intensity (1 PMT). Subsequently, the microarrays were analyzed using Mapix (Innopsys, Carbonne, France) microarray analysis software. As expected, the marker (streptavidin Cy3 and Cy5) and PC3 (mouse IgG,) showed signals. Table 3 below describes the name, structure and molecular weight of each GAG in the microarray.

TABLE 3
Name, structure and molecular weight of each GAG in the microarray

| | | | MW |
|---|---|---|---|
| ID | Name | Structure | (Da) |
| GAG1 | Hyaluronic Acid dp10 (HA10) | ΔGlcAβ1,3[GlcNAcβ1,4GlcAβ1,3]4 GlcNAc | 1,950 |
| GAG2 | Hyaluronic Acid dp12 (HA12) | ΔGlcAβ1,3[GlcNAcβ1,4GlcAβ1,3]5 GlcNAc | 2,350 |
| GAG3 | Hyaluronic Acid dp14 (HA14) | ΔGlcAβ1,3[GlcNAcβ1,4GlcAβ1,3]6 GlcNAc | 2,700 |
| GAG4 | Hyaluronic Acid dp16 (HA16) | ΔGlcAβ1,3[GlcNAcβ1,4GlcAβ1,3]7 GlcNAc | 3,150 |
| GAG5 | Hyaluronic Acid dp18 (HA18) | ΔGlcAβ1,3[GlcNAcβ1,4GlcAβ1,3]8 GlcNAc | 3,650 |
| GAG6 | Hyaluronic Acid dp20 (HA20) | ΔGlcAβ1,3[GlcNAcβ1,4GlcAβ1,3]9 GlcNAc | 3,900 |
| GAG7 | Hyaluronic Acid Polymer (HA93) | ΔGlcAβ1,3[GlcNAcβ1,4GlcAβ1,3]n GlcNAc | 93,000 |
| GAG8 | Heparin dp10 (H10) | ΔUA,2S-GlcNS,6S-[IdoUA,2S-GlcNS,6S]4 | 3,000 |
| GAG9 | Heparin dp12 (H12) | ΔUA,2S-GlcNS,6S-[IdoUA,2S-GlcNS,6S]5 | 3,550 |
| GAG10 | Heparin dp14 (H14 | ΔUA,2S-GlcNS,6S-[IdoUA,2S-GlcNS,6S]6 | 4,100 |
| GAG11 | Heparin dp16 (H16) | ΔUA,2S-GlcNS,6S-[IdoUA,2S-GlcNS,6S]7 | 4,650 |
| GAG12 | Heparin dp18 (H18) | ΔUA,2S-GlcNS,6S-[IdoUA,2S-GlcNS,6S]8 | 5,200 |
| GAG13 | Heparin dp20 (H20) | ΔUA,2S-GlcNS,6S-[IdoUA,2S-GlcNS,6S]9 | 5,750 |
| GAG14 | Heparin dp22 (H22) | ΔUA,2S-GlcNS,6S-[IdoUA,2S | 6,300 |
| | | GlcNS,6S]10 | |
| GAG15 | Heparin dp24 (H24) | ΔUA,2S-GlcNS,6S-[IdoUA,2S-GlcNS,6S]11 | 6,850 |
| GAG16 | Heparin dp30 (H30) | ΔUA,2S-GlcNS,6S-[IdoUA,2S- | 9,000 |
| | | GlcNS,6S]14 | |
| GAG17 | Chondroitin Sulphate Oligosaccharide dp10 | ΔUA-[GalNAc,6S or 4S-GlcA]4-GalNAc,6S | 2,480 |
| | (CSO10) | or 4S | |
| GAG18 | Chondroitin Sulphate Oligosaccharide dp12 | ΔUA-[GalNAc,6S or 4S-GlcA]5-GalNAc,6S | 2,976 |
| | (CSO12) | or 4S | |
| GAG19 | Chondroitin Sulphate Oligosaccharide dp14 | ΔUA-[GalNAc,6S or 4S-GlcA]6-GalNAc,6S | 3,472 |
| | (CSO14) | or 4S | |
| GAG20 | Chondroitin Sulphate Oligosaccharide dp16 | ΔUA-[GalNAc,6S or 4S-GlcA]7-GalNAc,6S | 3,968 |
| | (CSO16) | or 4S | |
| GAG21 | Chondroitin Sulphate Oligosaccharide dp18 | ΔUA-[GalNAc,6S or 4S-GlcA]8-GalNAc,6S | 4,464 |
| | (CSO18) | or 4S | |
| GAG22 | Chondroitin Sulphate Oligosaccharide dp20 | ΔUA-[GalNAc,6S or 4S-GlcA]9-GalNAc,6S | 4,960 |
| | (CSO20) | or 4S | |
| GAG23 | Chondroitin Sulphate D Oligosaccharide | ΔUA-[GalNAc,6S or 4S-GlcA+/−2S]4- | 2,480 |
| | dp10 (CSDO10) | GalNAc,6S | |
| GAG24 | Chondroitin Sulphate D Oligosaccharide | ΔUA-[GalNAc,6S or 4S-GlcA+/−2S]5- | 2,976 |
| | dp12 (CSDO12) | GalNAc,6S | |
| GAG25 | Chondroitin Sulphate D Oligosaccharide | ΔUA-[GalNAc,6S or 4S-GlcA+/−2S]6- | 3,472 |
| | dp14 (CSDO14) | GalNAc,6S | |
| GAG26 | Chondroitin Sulphate D Oligosaccharide | ΔUA-[GalNAc,6S or 4S-GlcA+/−2S]7- | 3,968 |
| | dp16 (CSDO16) | GalNAc,6S | |
| GAG27 | Chondroitin Sulphate D Oligosaccharide | ΔUA-[GalNAc,6S or 4S-GlcA+/−2S]8- | 4,464 |
| | dp18 (CSDO18) | GalNAc,6S | |
| GAG28 | Chondroitin Sulphate D Oligosaccharide | ΔUA-[GalNAc,6S or 4S-GlcA+/−2S]9- | 4,960 |
| | dp20 (CSDO20) | GalNAc,6S | |
| GAG29 | Dermatan Sulphate dp10 (DS10) | ΔUAβ1,3-GalNAc,4S-[IdoA-GalNAc,4S]4 | 2,480 |
| GAG30 | Dermatan Sulphate dp12 (DS12) | ΔUAβ1,3-GalNAc,4S-[IdoA-GalNAc,4S]5 | 2,976 |
| GAG31 | Dermatan Sulphate dp14 (DS14) | ΔUAβ1,3-GalNAc,4S-[IdoA-GalNAc,4S]6 | 3,472 |
| GAG32 | Dermatan Sulphate dp16 (DS16) | ΔUAβ1,3-GalNAc,4S-[IdoA-GalNAc,4S]7 | 3,968 |
| GAG33 | Dermatan Sulphate dp18 (DS18) | ΔUAβ1,3-GalNAc,4S-[IdoA-GalNAc,4S]8 | 4,464 |
| GAG34 | Dermatan Sulphate dp20 (DS20) | ΔUAβ1,3-GalNAc,4S-[IdoA-GalNAc,4S]9 | 4,960 |
| GAG35 | Heparan Sulphate Oligosaccharide dp10 | ΔUA2S-GlcNS-[GlcA-GlcNAc]3-IdoA- | 2,800 |
| | (Hep I, low and intermediate sulphation) | GlcNS | |
| GAG36 | Heparan Sulphate Oligosaccharide dp12 | ΔUA2S-GlcNS-[GlcA-GlcNAc]4-IdoA- | 3,500 |
| | (Hep I, low and intermediate sulphation) | GlcNS | |
| GAG37 | Heparan Sulphate Oligosaccharide dp14 | ΔUA2S-GlcNS-[GlcA-GlcNAc]5-IdoA- | 4,000 |
| | (Hep I, low and intermediate sulphation) | GlcNS | |
| GAG38 | Heparan Sulphate Oligosaccharide dp16 | ΔUA2S-GlcNS-[GlcA-GlcNAc]6-IdoA- | 4,400 |
| | (Hep I, low and intermediate sulphation) | GlcNS | |
| GAG39 | Heparan Sulphate Oligosaccharide dp18 | ΔUA2S-GlcNS-[GlcA-GlcNAc]7-IdoA- | 5,000 |
| | (Hep I, low and intermediate sulphation) | GlcNS | |
| GAG40 | Heparan Sulphate Oligosaccharide dp20 | ΔUA2S-GlcNS-[GlcA-GlcNAc]8-IdoA- | 5,400 |
| | (Hep I, low and intermediate sulphation) | GlcNS | |
| GAG41 | Heparan Sulphate Oligosaccharide dp10 | ΔUA-GlcNS-[IdoA+/−2S-GlcNS]3-IdoA- | 2,800 |
| | (Hep III, high sulphation) | GlcNAc | |
| GAG42 | Heparan Sulphate Oligosaccharide dp12 | ΔUA-GlcNS-[IdoA+/−2S-GlcNS]4-IdoA- | 3,500 |
| | (Hep III, high sulphation) | GlcNAc | |
| GAG43 | Heparan Sulphate Oligosaccharide dp14 | ΔUA-GlcNS-[IdoA+/−2S-GlcNS]5-IdoA- | 4,200 |
| | (Hep III, high sulphation) | GlcNAc | |
| GAG44 | Heparan Sulphate Oligosaccharide dp16 | ΔUA-GlcNS-[IdoA+/−2S-GlcNS]6-IdoA- | 4,800 |
| | (Hep III, high sulphation) | GlcNAc | |
| GAG45 | Heparan Sulphate Oligosaccharide dp18 | ΔUA-GlcNS-[IdoA+/−2S-GlcNS]7-IdoA- | 5,500 |
| | (Hep III, high sulphation) | GlcNAc | |
| GAG46 | Heparan Sulphate Oligosaccharide dp20 | ΔUA-GlcNS-[IdoA+/−2S-GlcNS]8-IdoA- | 6,200 |
| | (Hep III, high sulphation) | GlcNAc | |

FIGS. 1 and 2 shows the GAG structures used in the microarray whereas Table 4 below describes the name, structure and molecular weight of each HS used in the microarray.

TABLE 4
Name, structure and molecular weight of each heparan sulfate glycan
in the microarray
Table 3: Degree of sulfation chart for heparan sulfate array structures

| 24 Heparan Sulfate Glycans | | No. of Sugar | Sulfate Groups |
|---|---|---|---|
| No | Structure | Residues | per Disaccharide |
| HS001 | GlcNAc-GlcA-GlcNAc-GlcA | 4 | 0 |
| HS002 | GlcA-GlcNAc-GlcA-GlcNAc-GlcA | 5 | 0 |
| HS003 | GlcNAc-GlcA-GlcNAc-GlcA- GlcNAc-GlcA | 6 | 0 |
| HS004 | GlcA-GlcNAc-GlcA-GlcNAc-GlcA-GlcNAc-GlcA | 7 | 0 |
| HS005 | GlcNAc-GlcA-GlcNAc-GlcA-GlcNAc-GlcA-GlcNAc-GlcA | 8 | 0 |
| HS006 | GlcA-GlcNAc-GlcA-GlcNAc-GlcA-GlcNAc-GlcA-GlcNAc-GlcA | 9 | 0 |
| HS007 | GlcNS-GlcA-GlcNS-GlcA | 4 | 1 |
| HS008 | GlcA-GlcNS-GlcA-GlcNS-GlcA | 5 | 0.8 |
| HS009 | GlcNS-GlcA-GlcNS-GlcA-GlcNS-GlcA | 6 | 1 |
| HS010 | GlcA-GlcNS-GlcA-GlcNS-GlcA-GlcNS-GlcA | 7 | 0.9 |
| HS011 | GlcNS-GlcA-GlcNS-GlcA-GlcNS-GlcA-GlcNS-GlcA | 8 | 1 |
| HS012 | GlcA-GlcNS-GlcA-GlcNS-GlcA-GlcNS-GlcA-GlcNS-GlcA | 9 | 0.9 |
| HS013 | GlcA-GlcNS-GlcA-GlcNS-GlcA-GlcNS-GlcA-GlcNS6S-GlcA | 9 | 1.1 |
| HS014 | GlcA-GlcNS-GlcA-GlcNS-GlcA-GlcNS6S-GlcA-GlcNS6S-GlcA | 9 | 1.3 |
| HS015 | GlcA-GlcNS-GlcA-GlcNS6S-GlcA-GlcNS6S-GlcA-GlcNS6S-GlcA | 9 | 1.6 |
| HS016 | GlcA-GlcNS6S-GlcA-GlcNS6S-GlcA-GlcNS6S-GlcA-GlcNS6S-GlcA | 9 | 1.8 |
| HS017 | GlcNS-GlcA-GlcNS-GlcA-GlcNS-IdoA2S-GlcNS-GlcA | 8 | 1.3 |
| HS018 | GlcNS-GlcA-GlcNS-IdoA2S-GlcNS-IdoA2S-GlcNS-GlcA | 8 | 1.5 |
| HS019 | GlcNAc-GlcA-GlcNS-IdoA2S-GlcNS-IdoA2S-GlcNS-GlcA | 8 | 1.3 |
| HS020 | GlcNS6S-GlcA-GlcNS6S-GlcA-GlcNS6S-IdoA2S-GlcNS6S-GlcA | 8 | 2.3 |
| HS021 | GlcNS6S-GlcA-GlcNS6S-IdoA2S-GlcNS6S-IdoA2S-GlcNS6S-GlcA | 8 | 2.5 |
| HS022 | GlcNAc6S-GlcA-GlcNS6S-IdoA2S-GlcNS6S-IdoA2S-GlcNS6S-GlcA | 8 | 2.3 |
| HS023 | GlcNS6S-GlcA-GlcNS3S6S-IdoA2S-GlcNS6S-GlcA | 6 | 2.7 |
| HS024 | GlcNAc6S-GlcA-GlcNS3S6S-IdoA2S-GlcNS6S-IdoA2S-GlcNS6S-GlcA | 8 | 2.5 |

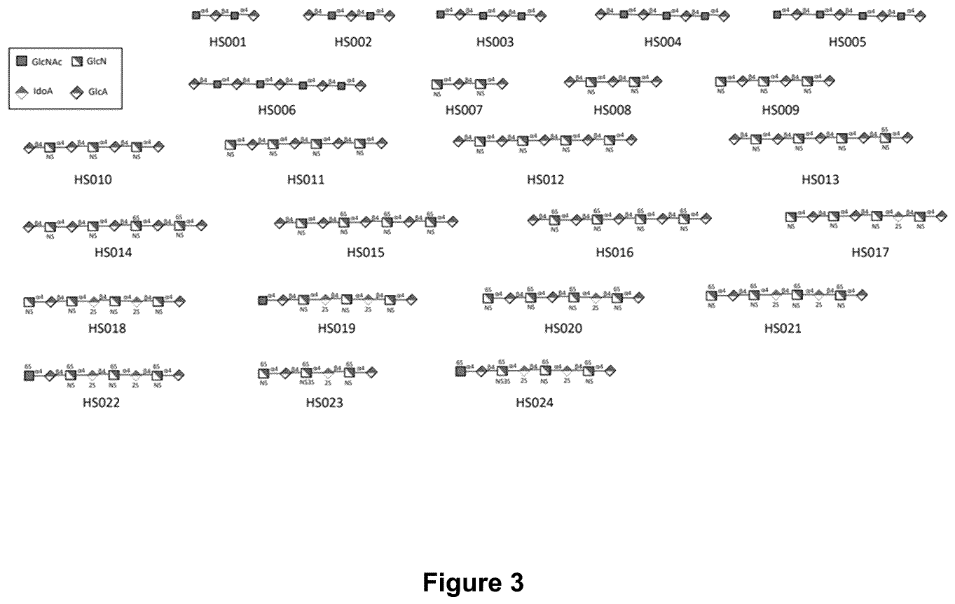

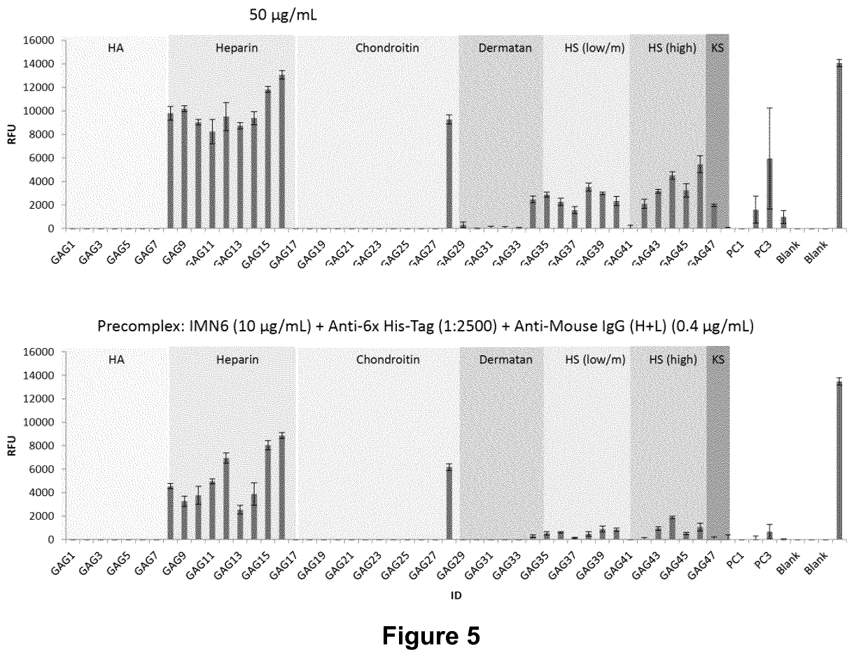

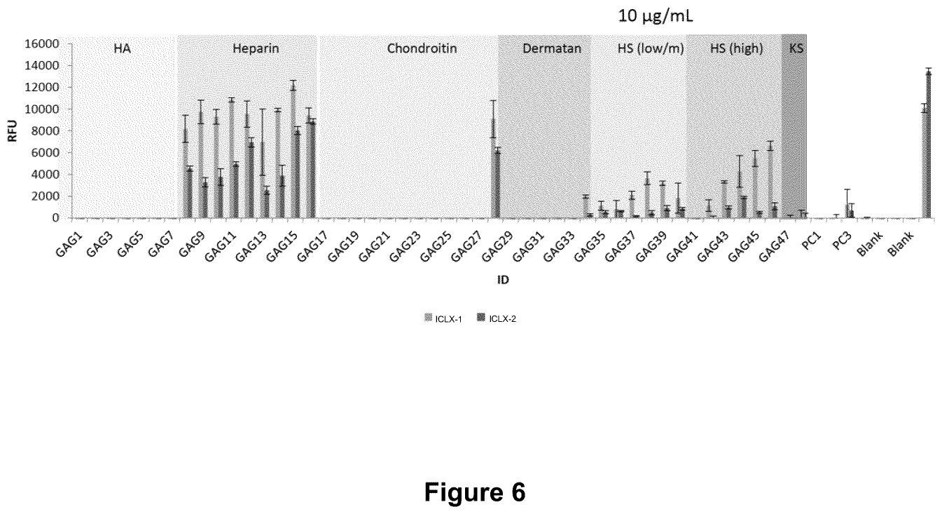

FIG. 3 depicts a representation of the HS structures used in the microarray. The results shown in FIGS. 4-6 indicate that both ICLX-1 scFv and ICLX-2 scFv bind to a variety of sulfated glycan structures including Hep, HS, CSD, and DS. At the highest concentration tested (50 μg/mL), they also bind to KS. Of the two, ICLX-1 scFv consistently exhibited stronger binding signals compared to ICLX-2 scFv. Overall, glycan binding strength appeared to be positively correlated with longer chain lengths of Hep and HS as well as the degree of sulfation of the glycans. This is consistent with an electrostatic binding mechanism.

ICLX-1 scFv and ICLX-2 scFv binding to CS was limited to the highest dp of CSD, a CS chain that is primarily sulfated at the C2 position of GlcA and the C4 position of GalNAc. CSD has a much higher negative charge density compared to either CSA or CSC (which are primarily only monosulfated at the C4 and C6 positions of GalNAc, respectively), and the 2-O-sulfation of the GlcA resembles the 2-O-sulfation of IdoA found in Hep.

ICLX-1 scFv and ICLX-2 scFv both bound to DS with increasing affinity for the longer chain lengths, which is again consistent with an electrostatic binding mechanism. While DS has a sulfate density similar to CSA/CSC (one sulfo group per disaccharide unit, found at the GalNAc), it also bears similarity to Hep in that it has IdoA in its disaccharide repeat as most commonly found in Hep, unlike CS which has GlcA (differing by epimerization at the C-5 position). These results indicate that both ICLX-1 scFv and ICLX-2 scFv have specificity for Hep and Hep-like GAGs.

The apparent K_{d} of ICLX-1 scFv (MW 28.08 kDa) for various GAGs was calculated in silico using MotifFinder software (Klamer and Haab, 2021) as follows:
3.52 μg/mL or 125 nM for GAG15 (Heparin, dp24)
5.59 μg/mL or 199 nM for GAG28 (Chondroitin sulfate D, dp20)
9.44 μg/mL or 336 nM for GAG46 (Hep III high sulfation, dp20)
10.8 μg/mL or 385 nM for GAG38 (Hep I low and intermediate sulfation, dp16)
12.1 μg/mL or 431 nM for GAG34 (Dermatan sulfate, dp20)

The apparent K_{d} of ICLX-2 scFv (MW 28.11 kDa) for various GAGs was calculated in silico using MotifFinder software as follows:
6.63 μg/mL or 235 nM for GAG15 (Heparin dp24)
7.07 μg/mL or 252 nM for GAG28 (Chondroitin sulfate D, dp20)
34.8 μg/mL or 1.24 μM for GAG46 (Hep III high sulfation, dp20)
486 μg/mL or 17.3 μM for GAG38 (Hep I low and intermediate sulfation, dp16)
532 μg/mL or 18.9 μM for GAG34 (Dermatan sulfate, dp20)

These results are summarized in Table 5 below for an easier comparison of the two synthesized antibodies.

TABLE 5
K_{d} of ICLX-1 scFv (MW 28.08 kDa) and ICLX-2 scFv (MW 28.11 kDa)
for various GAGs as calculated using MotifFinder software

| | | Degree | | |
|---|---|---|---|---|
| | | of poly- | | |
| Microarray | | merization | K_{d} | |
| identifier | GAG name | (dp) | ICLX-1 | ICLX-2 |
| GAG15 | Heparin (Hep) | 24 | 125 nM | 235 nM |
| GAG28 | Chondroitin sulfate D | 20 | 199 nM | 252 nM |
| | (CSD) | | | |
| GAG46 | Heparan sulfate III | 20 | 336 nM | 1.24 μM |
| | (HSIII) | | | |
| | (high sulfation) | | | |
| GAG38 | Heparan sulfate I (HSI) | 16 | 385 nM | 17.3 μM |
| | (low and intermediate | | | |
| | sulfation) | | | |
| GAG34 | Dermatan sulfate | 20 | 431 nM | 18.9 μM |

Both ICLX-1 scFv and ICLX-2 scFv bind most strongly to long-chain Hep. Thus, these may be regarded as primarily anti-Hep antibodies that cross-react with other sulfated GAG species, including HS.

Substituting valine for alanine in the final position of VHCDR3 (Kabat) had an unfavorable effect on the K_{d} of ICLX-2 scFv for specific GAGs, in particular low/intermediate sulfation HS and OS. The order in which ICLX-2 scFv binds to specific GAGs is the same as for ICLX-1 scFv.

FIGS. 7 and 8 show the results for ICLX-1 scFv and ICLX-2 scFv binding to the HS microarray. The apparent K_{d} of ICLX-1 scFv (MW 28.08 kDa) for various HSs was calculated in silico using MotifFinder software (Klamer and Haab, 2021) as follows:
11.21 μg/mL or 399.2 nM for HS016 (nonasaccharide, 1.8 sulfate groups per disaccharide)
14.87 μg/mL or 529.6 nM for HS021 (octasaccharide, 2.5 sulfate groups per disaccharide)
13.32 μg/mL or 474.4 nM for HS022 (octasaccharide, 2.3 sulfate groups per disaccharide)
21.79 μg/mL or 776.0 nM for HS023 (hexasaccharide, 2.7 sulfate groups per disaccharide)
12.77 μg/mL or 454.8 nM for HS024 (octasaccharide, 2.5 sulfate groups per disaccharide)

The apparent Kd of ICLX-2 scFv (MW 28.11 kDa) for various HSs was calculated in silico using MotifFinder software as follows:
16.31 μg/mL or 580.2 nM for HS016 (nonasaccharide, 1.8 sulfate groups per disaccharide)
29.68 μg/mL or 1.056 μM for HS021 (octasaccharide, 2.5 sulfate groups per disaccharide)
257.9 μg/mL or 9.176 μM for HS022 (octasaccharide, 2.3 sulfate groups per disaccharide)
62.90 μg/mL or 2.238 μM for HS023 (hexasaccharide, 2.7 sulfate groups per disaccharide)
28.22 μg/mL or 1.004 μM for HS024 (octasaccharide, 2.5 sulfate groups per disaccharide)

These results are summarized in Table 6 below for an easier comparison of the two synthesized antibodies.

TABLE 6
Kd of ICLX-1 scFv (MW 28.08 kDa) and ICLX-2 scFv (MW 28.11 kDa)
for various HSs as calculated using MotifFinder software

| | | Degree | | |
|---|---|---|---|---|
| | | of poly- | | |
| Microarray | | merization | | |
| identifier | GAG name | (dp) | K_{d} | |
| HS016 | Heparan sulfate | 9 | 399.2 nM | 580.2 nM |
| | (1.8 sulfate groups per | | | |
| | disaccharide) | | | |
| HS021 | Heparan sulfate | 8 | 529.6 nM | 1.056 μM |
| | (2.5 sulfate groups per | | | |
| | disaccharide) | | | |
| HS022 | Heparan sulfate | 8 | 474.4 nM | 9.176 μM |
| | (2.3 sulfate groups per | | | |
| | disaccharide) | | | |
| HS023 | Heparan sulfate | 6 | 776.0 nM | 2.238 μM |
| | (2.7 sulfate groups per | | | |
| | disaccharide) | | | |
| HS024 | Heparan sulfate | 8 | 454.8 nM | 1.004 μM |
| | (2.5 | | | |
| | sulfates/disaccharide) | | | |

Binding of both ICLX-1 scFv and ICLX-2 scFv to HS has an absolute requirement for sulfation. Neither antibody bound to unsulfated GlcNAc-GlcA oligomers of any length. Thus, both ICLX-1 scFv and ICLX-2 scFv bind only to sulfated regions of HS molecules, and in proportion to the degree of sulfation was thus concluded.

The absence of a common motif among the many different GAG species that bind to ICLX-1 scFv and ICLX-2 scFv, plus the tendency for binding to increase with increasing sulfation and dp, implicates negative charge density and electrostatic forces as the primary determinants of binding, rather than a ‘lock-and-key” mechanism. This is in contrast with anti-HS antibodies such as AO4B08 and HS4E4, which were developed using phage display technology, and which allegedly recognize well-characterized short oligosaccharide sequences (Kurup, et al., 2007).

Antibody HS3B7 is another anti-HS antibody which was developed using phage display. Thompson et al. (2009) modeled the surface electrostatic potential of antibodies HS4E4 and HS3B7 and concluded that they both possess highly basic VHCDR3 (Kabat) and surrounding surfaces, and that the maximum size HS chain they could accommodate was dp10. They opined that “Geometrically, for a heparin oligosaccharide of dp12 or longer to bind to HS3B7, the polysaccharide would have to wrap around the antibody, which is unlikely given the relatively limited flexibility of the heparin glycosidic linkage”. This is in marked contrast with ICLX-1 scFv and ICLX-2 scFv, which can easily bind to HS with dp24. The VHCDR3 (Kabat) of antibody HS3B7 is SRKTRKPFMRK, which is only 4 amino acids shorter than the VHCDR3 (Kabat) of both ICLX-1 scFv and ICLX-2 scFv. This difference in size is not enough to explain the more than doubling of capacity of the latter in terms of HS chain length. Clearly, electrostatic forces involving more than one CDR region must be responsible for ICLX-1 scFv and ICLX-2 scFv binding to long-chain sulfated GAGs.

### Example 3: Side-by-Side Comparison of ICLX-1 scFv and ICLX-3 scFv in GAG and HS Microarrays

In this example, ICLX-1 scFv and ICLX-3 scFv binding to a GAG microarray was compared.

ICLX-3 scFv is representative of SEQ ID NO:14 of WO97/44461, which means that it is functionally equivalent to NovoMAb-G2 scFv. The latter is an unconjugated antibody fragment that has previously been shown to have an anti-cancer effect in tumor xenograft models as well as a Phase I/II clinical trial. Until now, the mechanism of action of NovoMAb-G2 scFv was unclear.

Both ICLX-2 scFv and ICLX-3 scFv have a valine in the terminal position of VHCDR3 (Kabat), and as described in in Example 2, the latter had an unfavorable effect on binding to sulfated GAGs (in particular, low/intermediate sulfation HS, and DS). Unlike ICLX-2 scFv, however, ICLX-3 scFv has an aspartic acid in the initial position of VHCDR3 (Kabat), which would be expected to have an additional unfavorable effect on binding due to repulsive forces with negatively charged sulfate and carboxylate groups on Hep and Hep-like GAGs.

### Method

The microarray setup and assay methodology are as described in Example 2. Briefly, the microarray was pretreated with Glycan Array Blocking Buffer (GABB) at room temperature for one hour. After blocking, a pre-complexed mixture of ICLX-1 scFv or ICLX-3 scFv, anti-his tag mAb, and anti-mouse IgG (H+L) AF555 was added to the microarray and incubated at room temperature for 1 hour. The precomplexed mixture initially contained ICLX-1 scFv or ICLX-3 scFv at 10 μg/mL or 50 μg/mL concentration, anti-his tag mAb at 1:500 dilution, and anti-mouse IgG (H+L) AF555 at 2 μg/mL concentration. This mixture was then diluted by factors of 5 to 6 times. After incubation at room temperature for 1 hour, the microarray was washed and scanned at 532 nm using high laser intensity (1 PMT). Subsequently, the microarrays were analyzed using Mapix (Innopsys, Carbonne, France) microarray analysis software.

### Results

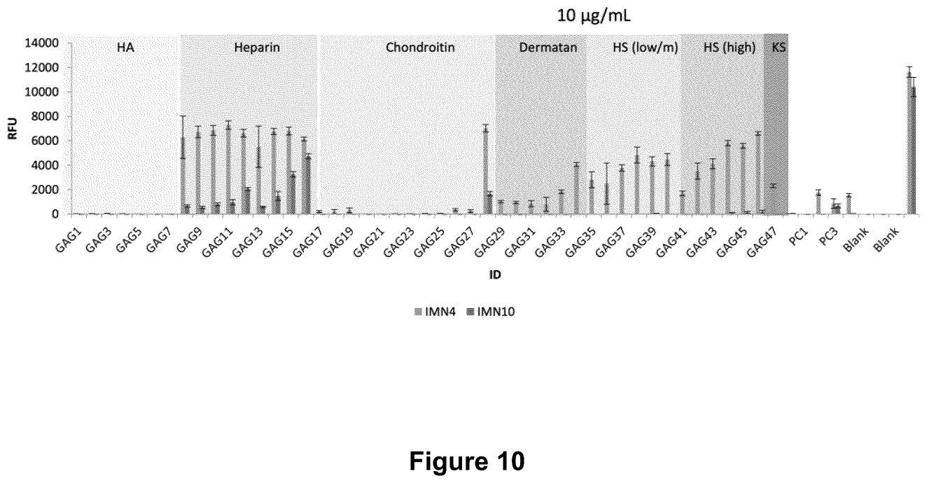

The GAG microarray results are shown in FIG. 10 and show consistency for ICLX-1 scFv with the results obtained in Example 2. The GAG microarray results for ICLX-3 scFv shown in FIG. 9 tell a different story. What stands out is that at the highest concentration tested (50 μg/mL), ICLX-3 scFv binding to CSD is on par with binding to Hep. Binding to high sulfation HS barely registers on the microarray. At the next lower concentration (10 μg/mL), ICLX-3 scFv binding to HS has all but dropped down to the level of background noise.

In retrospect, this may shed some light on why NovoMAb-G2 scFv was once presumed to bind only to CSA. If it is assumed that: 1) NovoMAb-G2 scFv was only previously tested against GAGs in ELISA, and 2) it was never specifically tested against Hep, then the only GAG that it consistently binds to is a form of CS (i.e., CSD). Without any further testing against specific forms of CS, it would seem reasonable to assume that NovoMAb-G2 scFv binds to CSA, since the latter is the most common form of CS on human cells. In fact, neither ICLX-1 scFv, ICLX-2 scFv, nor ICLX-3 scFv (which is functionally equivalent to NovoMAb-G2 scFv) bind to CSA when tested in a GAG microarray. Moreover, the prior art does not disclose any direct evidence (either by ELISA or any other method) that NovoMAb-G2 scFv binds to CSA.

The apparent K_{d} of ICLX-3 scFv (MW 28.23 kDa) for various HSs was calculated in silico using MotifFinder software as follows:
52.5 μg/mL or 1.860 μM for GAG15 (Heparin dp24)
160 μg/mL or 5.65 μM for GAG28 (Chondroitin sulfate D, dp20)
987 μg/mL or 35.0 μM for GAG46 (Hep III high sulfation, dp20)

Like ICLX-1 scFv and ICLX-2 scFv, ICLX-3 scFv binds most strongly to long-chain Hep. It may thus be regarded as primarily an anti-Hep antibody that cross-reacts with CSD dp20 and high sulfation HS. No binding to low/intermediate sulfation HS or DS was ascertained using MotifFinder software.

ICLX-3 scFv has the identical CDR regions as NovoMAb-G2 scFv. These results thus indicate a previously unrecognized reactivity of NovoMAb-G2 scFv with high sulfation HS, however, the level of reactivity is quite low (the apparent K_{d} for ICLX-3 scFv is ~10× that of ICLX-1 scFv). Reactivity with low/intermediate sulfation HS and with DS was undetectable, as confirmed by MotifFinder software. No reactivity of ICLX-3 scFv with CSA/C was observed, thus prior art claims that NovoMAb-G2 scFv reacts with CSA are invalid.

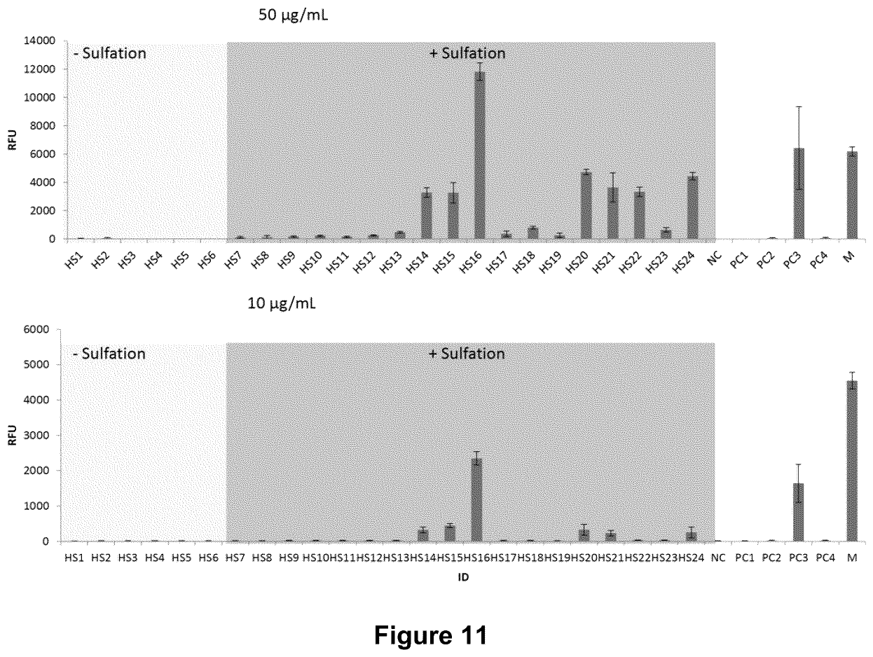

FIG. 11 shows the results of ICLX-3 scFv binding to the HS microarray.

Binding of ICLX-3 scFv to HS has an absolute requirement for sulfation. ICLX-3 scFv did not bind to unsulfated GlcNAc-GlcA oligomers of any length. Thus, it is concluded that ICLX-3 scFv binds only to sulfated regions of HS molecules, and in proportion to the degree of sulfation.

The apparent K_{d} of ICLX-3 scFv (MW 28.23 kDa) for various HSs was calculated in silico using MotifFinder software as follows:
209.5 μg/mL or 7.420 μM for HSO16 (nonasaccharide, 1.8 sulfate groups per disaccharide)
258.0 μg/mL or 9.139 μM for HS021 (octasaccharide, 2.5 sulfate groups per disaccharide)
106.2 μg/mL or 3.762 μM for HS022 (octasaccharide, 2.3 sulfate groups per disaccharide)
557.5 μg/mL or 19.75 μM for HS023 (hexasaccharide, 2.7 sulfate groups per disaccharide)
210.4 μg/mL or 7.453 μM for HS024 (octasaccharide, 2.5 sulfate groups per disaccharide)

The results for ICLX-3 are summarized in Table 7 and 8 below.

TABLE 7
K_{d} of ICLX-3 scFv (MW 28.23 kDa) for various GAGs as calculated
using MotifFinder software

| | | Degree of | |
|---|---|---|---|
| Microarray | | polymerization | |
| identifier | GAG name | (dp) | K_{d} |
| GAG15 | Heparin (Hep) | 24 | 1.860 nM |
| GAG28 | Chondroitin sulfate D (CSD) | 20 | 5.65 μM |
| GAG46 | Heparan sulfate III | 20 | 35.0 μM |
| | (high sulfation) | | |

TABLE 8
Kd of ICLX-3 scFv (MW 28.23 kDa) for various HSs as calculated
using MotifFinder software

| | | Degree of | |
|---|---|---|---|
| Microarray | | polymerization | |
| identifier | GAG name | (dp) | K_{d} |
| HS016 | Heparan sulfate | 9 | 7.420 μM |
| | (1.8 sulfate groups per | | |
| | disaccharide) | | |
| HS021 | Heparan sulfate | 8 | 9.139 μM |
| | (2.5 sulfate groups per | | |
| | disaccharide) | | |
| HS022 | Heparan sulfate | 8 | 3.762 μM |
| | (2.3 sulfate groups per | | |
| | disaccharide) | | |
| HS023 | Heparan sulfate | 6 | 19.75 μM |
| | (2.7 sulfate groups per | | |
| | disaccharide) | | |
| HS024 | Heparan sulfate | 8 | 7.453 μM |
| | (2.5 sulfates/disaccharide) | | |

Clearly, ICLX-3 scFv behaves very differently from ICLX-1 scFv, notwithstanding a difference of only 2 amino acids in their respective VHCDR3 (Kabat) regions.

Given ICLX-3 scFv's relatively poor binding to high sulfation HS, and the central role that HS plays as a co-receptor for growth factor binding to tumor cells, how can the effectiveness of NovoMAb-G2 scFv in xenograft models and in a Phase I/II clinical trial be explained? The answer to this question may lie with NovoMAb-G2 scFv's behavior in the mildly acidic tumor microenvironment (TME) (pH ~6.0; Hao, et al. 2018).

### Example 4: Testing of ICLX-3 scFv Under Mildly Acidic Conditions

In the 1920's, Otto Warburg observed that even in the presence of adequate oxygen, tumor cells preferentially turn to fermentation of lactic acid for an energy supply. The so-called Warburg Effect leads to a constitutively acidic TME (Liberti and Locasale, 2016). ICLX-3 scFv binding to HS under mildly acidic conditions was therefore tested.

Of the three antibodies related to H11, ICLX-3 scFv has the lowest affinity for high sulfation Hep. This is the result of a valine for alanine substitution and an extra aspartic acid in VHCDR3 (Kabat). However, ICLX-1 scFv, ICLX-2 scFv, and ICLX-3 scFv all have a histidine in VHCDR1 (Kabat) and VHCDR3 (Kabat), and that histidine becomes protonated as the pH nears 6.0. Thus, under mildly acidic conditions, a qualitative improvement in the CW motifs of ICLX-3 scFv is expected, as histidine behaves more like a basic amino acid.

The heavy and light chain CDR sequences (Kabat) of ICLX-3 scFv (NovoMAb-G2 scFv), with their corresponding CW motifs, are represented in Table 9. Histidine residues are indicated as B.

TABLE 9
Corresponding CW motifs of ICLX-3 scFv

| ICLX-3 | CDR1 | CDR2 | CDR3 |
|---|---|---|---|
| VH | SFAMH | VISYDGSTKYYADSVKG | DQSLLGDYDHYYGLDV |
| | -XXXB | XX--OX-XB--XO-XBX | O--XXXO-OB--XXOX |
| VL | RASQSVSSSYLA | GASTRAT | QQYGSSPOTPQIT |
| | BX---X----XX | XX-XBXX | ---X----X--XX |

### Method

The microarray setup and assay methodology are as described in Examples 2 and 3. Briefly, the microarray was pretreated with Glycan Array Blocking Buffer (GABB) at room temperature for one hour. After blocking, a pre-complexed mixture of ICLX-3 scFv, anti-his tag mAb, and anti-mouse IgG (H+L) AF555 at pH 7.4, 6.8 or 6.0 was added to the microarray and incubated at room temperature for 1 hour. The precomplexed mixture initially contained ICLX-3 scFv at 2 μg/mL or 50 μg/mL concentration, anti-his tag mAb at 1:500 dilution, and anti-mouse IgG (H+L) AF555 at 2 μg/mL concentration. This mixture was then diluted by factors of 5 to 6 times. After incubation at room temperature for 1 hour, the microarray was washed and scanned at 532 nm using high laser intensity (1 PMT). Subsequently, the microarrays were analyzed using Mapix (Innopsys, Carbonne, France) microarray analysis software.

### Results

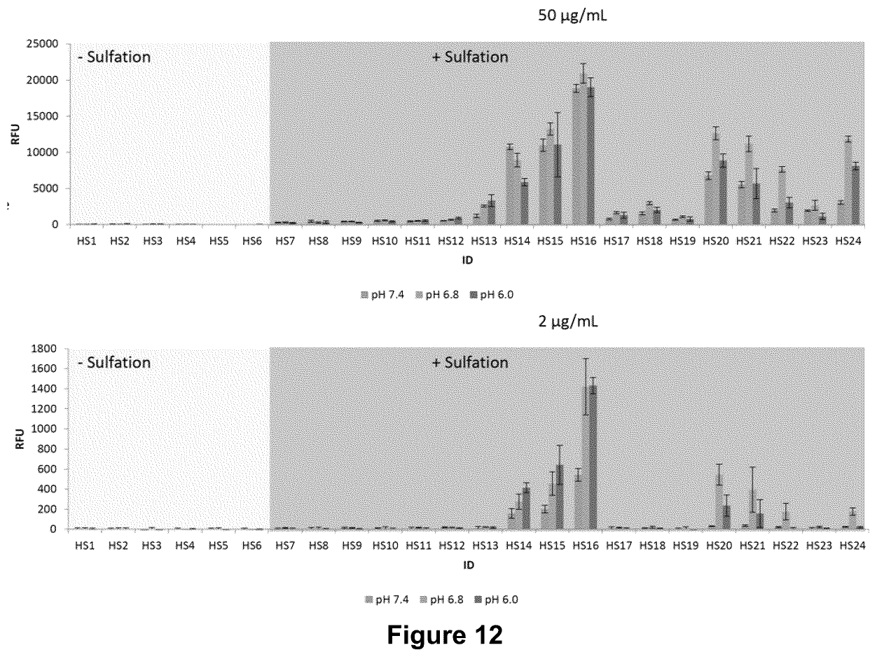

FIG. 12 shows the binding of ICLX-3 scFv to the HS microarray under different pH conditions.

The following table summarizes the effects of pH on assumed Kd (μM) for ICLX-3 scFv binding to high sulfation (i.e., >2 sulfate groups per disaccharide) heparan sulfate structures, as estimated in silico using MotifFinder software:

TABLE 10
K_{d} of ICLX-3 scFv (MW 28.23 kDa) for various HSs scFv under
mildly acidic conditions as calculated using MotifFinder software

| | | Degree | | | |
|---|---|---|---|---|---|
| | | of poly- | | | |
| Microarray | | merization | K_{d} (μM) | | |
| identifier | GAG name | (dp) | pH 7.4 | pH 6.8 | pH 6.0 |
| HS020 | Heparan sulfate | 8 | 1.417 | 0.4453 | 0.3631 |
| | (2.3 sulfate groups | | | | |
| | per disaccharide) | | | | |
| HS021 | Heparan sulfate | 8 | 1.333 | 6.858 | 0.4141 |
| | (2.5 sulfate groups | | | | |
| | per disaccharide) | | | | |
| HS022 | Heparan sulfate | 8 | 14.71 | 1.836 | 0.3325 |
| | (2.3 sulfate groups | | | | |
| | per disaccharide) | | | | |
| HS023 | Heparan sulfate | 6 | 12.44 | 15.34 | 0.7538 |
| | (2.7 sulfate groups | | | | |
| | per disaccharide) | | | | |
| HS024 | Heparan sulfate | 8 | 9.901 | 0.9493 | 0.4052 |
| | (2.5 sulfates/ | | | | |
| | disaccharide) | | | | |

These results indicate that at pH 6.8, ICLX-3 scFv binding to HS20, HS021, HS22, and HS24 is significantly enhanced relative to pH 7.4; especially at lower concentrations (i.e., ~2 μg/mL). What these 4 HS molecules all have in common is an octasaccharide structure and >2 sulfate groups per disaccharides.

ICLX-3 scFv is an example of a pH-responsive antibody (Klaus and Deshmukh, 2021), whose potential anti-tumor effect may be enhanced in the TME.

### Example 5: Electrostatic Improvement of the ICLX-1 VHCDR3

The VHCDR3 (Kabat) of ICLX-1 scFv features a single histidine residue (see SEQ ID NO:1). As was shown in Example 4, lowering the pH to 6.8 improves the electrostatic potential of histidine, and thus binding of ICLX-3 scFv to GAGs. The purpose of this experiment was to determine the effect of replacing the histidine of the ICLX-1 VHCDR3 (Kabat) with a more basic amino acid, such as arginine or lysine, on binding to a GAG microarray.

Antibody ICLX-4 scFv is identical to ICLX-1 scFv except for the substitution of R(Arg) for H(His) in VHCDR3 (Kabat); and antibody ICLX-5 scFv is identical to ICLX-1 scFv except for the substitution of K(Lys) for H(His) in VHCDR3 (Kabat). Both ICLX-4 scFv and ICLX-5 scFv were prepared by Sino Biological US Inc. and tested against GAG and HS microarrays by Z Biotech, LLC.

### Method

The microarray setup and assay methodology are as described in Examples 2-4. Briefly, the microarray was pretreated with Glycan Array Blocking Buffer (GABB) at room temperature for one hour. After blocking, a pre-complexed mixture of ICLX-4 scFv or ICLX-5 scFv, anti-his tag mAb, and anti-mouse IgG (H+L) AF555 was added to the microarray and incubated at room temperature for 1 hour. The precomplexed mixture initially contained ICLX-4 or ICLX-5 scFv at 10 μg/mL or 50 μg/mL concentration, anti-his tag mAb at 1:500 dilution, and anti-mouse IgG (H+L) AF555 at 2 μg/mL concentration. This mixture was then diluted by factors of 5 to 6 times. After incubation at room temperature for 1 hour, the microarray was washed and scanned at 532 nm using high laser intensity (1 PMT). Subsequently, the microarrays were analyzed using Mapix (Innopsys, Carbonne, France) microarray analysis software.

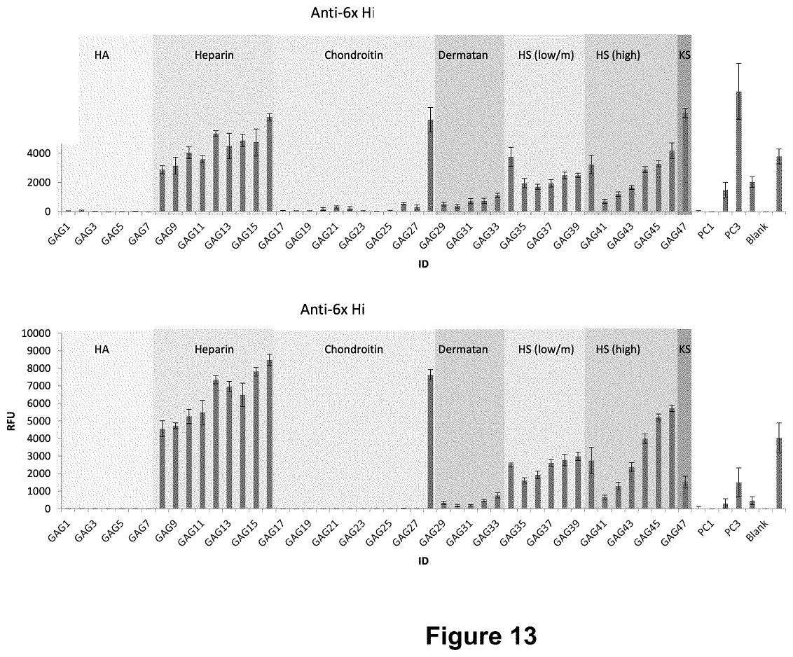

The results for ICLX-4 scFv are shown in FIG. 13.

The apparent K_{d} of ICLX-4 scFv (MW 28.10 kDa) for various GAGs was calculated in silico using MotifFinder software as follows:
0.974 μg/mL or 35 nM for GAG15 (Heparin, dp24)
1.29 μg/mL or 45.9 nM for GAG28 (Chondroitin sulfate D, dp20)
1.56 μg/mL or 55.5 nM for GAG46 (Hep III high sulfation, dp20)
1.62 μg/mL or 57.7 nM for GAG38 (Hep I low and intermediate sulfation, dp16)
6.25 μg/mL or 222 nM for GAG34 (Dermatan sulfate, dp20)

The apparent affinity of ICLX-4 scFv for GAG15 (Heparin, dp24) was the highest of any GAG species tested. ICLX-4 scFv may thus be regarded as primarily an anti-Hep antibody that cross-reacts with other sulfated GAG species, including HS.

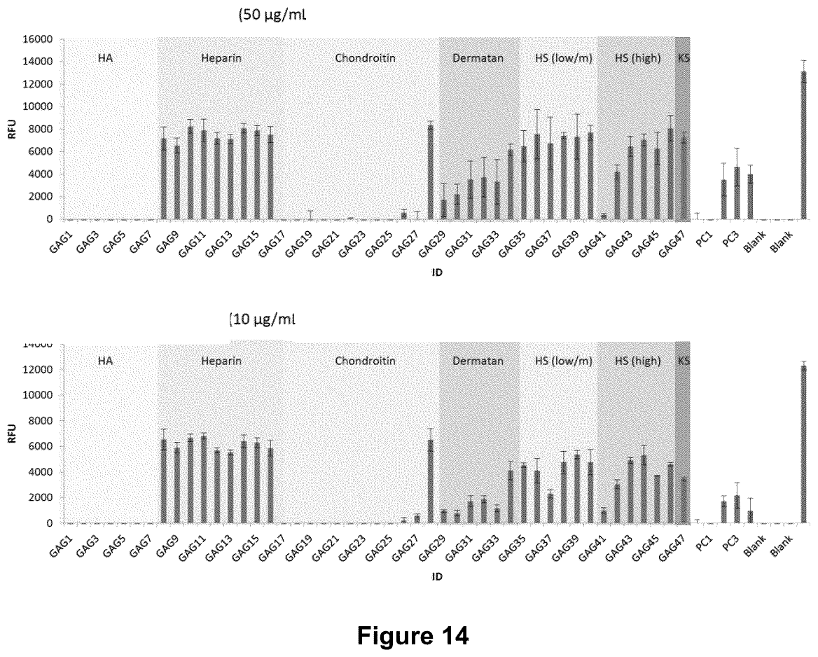

The results for ICLX-5 scFv are shown in FIG. 14.

The apparent Kd of ICLX-5 scFv (MW 28.08 kDa) for various GAGs was calculated in silico using MotifFinder software as follows:
4.86 μg/mL or 173 nM for GAG15 (Heparin dp24)
5.21 μg/mL or 186 nM for GAG28 (Chondroitin sulfate D, dp20)
7.54 μg/mL or 269 nM for GAG34 (Dermatan sulfate, dp20)
7.78 μg/mL or 277 nM for GAG38 (Hep I low and intermediate sulfation, dp16)
9.25 μg/mL or 329 nM for GAG46 (Hep III high sulfation, dp20)

Table 11 summarizes the binding of ICLX-4 scFv and ICLX-5 scFv to various GAGs.

TABLE 11
K_{d} of ICLX-4 scFv (MW 28.10 kDa) and ICLX-5 scFv (MW 28.08
kDa) for various GAGs as calculated using MotifFinder software

| | | Degree of | K_{d} | |
|---|---|---|---|---|
| Microarray | | polymerization | ICLX-4 | ICLX-5 |
| identifier | GAG name | (dp) | scFv | scFv |
| GAG15 | Heparin (Hep) | 24 | 35 nM | 173 nM |
| GAG28 | Chondroitin sulfate D | 20 | 45.9 nM | 186 nM |
| | (CSD) | | | |
| GAG46 | Heparan sulfate III | 20 | 55.5 nM | 329 nM |
| | (high sulfation) | | | |
| GAG38 | Heparan sulfate I (HepI) | 16 | 57.7 nM | 277 nM |
| | (low and intermediate | | | |
| | sulfation) | | | |
| GAG34 | Dermatan sulfate | 20 | 222 nM | 269 nM |

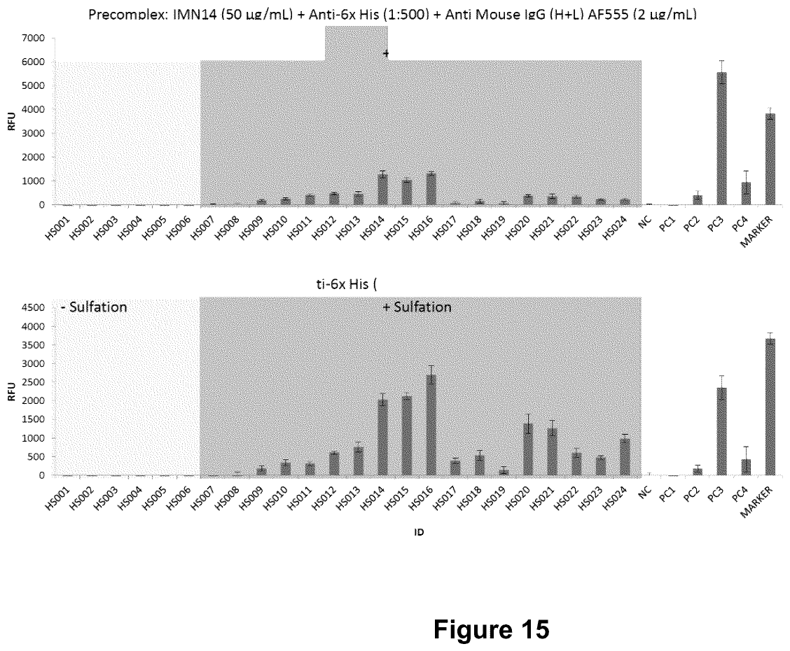

Results for ICLX-4 scFv binding to the HS microarray are provided in FIG. 15.

Binding of ICLX-4 scFv to HS has an absolute requirement for sulfation. ICLX-4 scFv did not bind to unsulfated GlcNAc-GlcA oligomers of any length. Thus, it can be concluded that ICLX-4 scFv binds only to sulfated regions of HS molecules, and in proportion to the degree of sulfation.

The apparent K_{d} of ICLX-4 scFv (MW 28.10 kDa) for various HSs was calculated in silico using MotifFinder software as follows:
0.1065 μg/mL or 3.790 nM for HSO16 (nonasaccharide, 1.8 sulfate groups per disaccharide)
0.08271 μg/mL or 2.943 nM for HSO21 (octasaccharide, 2.5 sulfate groups per disaccharide)
0.7630 μg/mL or 27.15 nM for HS022 (octasaccharide, 2.3 sulfate groups per disaccharide)
0.07494 μg/mL or 2.667 nM for HS023 (hexasaccharide, 2.7 sulfate groups per disaccharide)
0.08269 μg/mL or 2.943 nM for HS024 (octasaccharide, 2.5 sulfate groups per disaccharide)

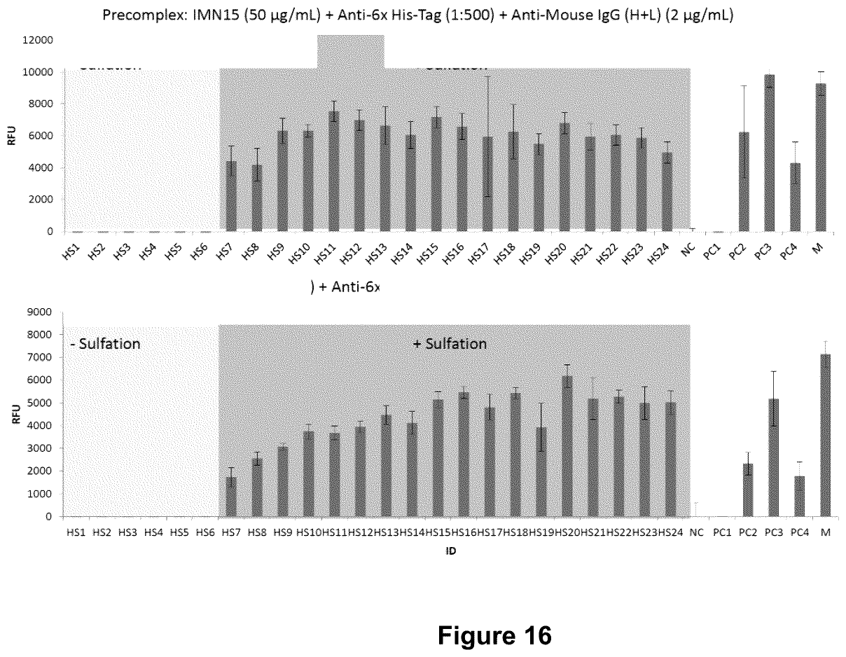

Results for ICLX-5 scFv binding to the HS microarray are provided in FIG. 16.

Binding of ICLX-5 scFv to HS has an absolute requirement for sulfation. ICLX-5 scFv did not bind to unsulfated GlcNAc-GlcA oligomers of any length. Thus, it can be concluded that ICLX-5 scFv binds only to sulfated regions of HS molecules, and in proportion to the degree of sulfation.

The apparent K_{d} of ICLX-5 scFv (MW 28.08 kDa) for various HSs was calculated in silico using MotifFinder software as follows:
3.756 μg/mL or 133.9 nM for HS016 (nonasaccharide, 1.8 sulfate groups per disaccharide)
2.890 μg/mL or 102.9 nM for HS021 (octasaccharide, 2.5 sulfate groups per disaccharide)
3.268 μg/mL or 116.4 nM for HS022 (octasaccharide, 2.3 sulfate groups per disaccharide)
3.558 μg/mL or 126.7 nM for HS023 (hexasaccharide, 2.7 sulfate groups per disaccharide)
2.507 μg/mL or 89.28 nM for HS024 (octasaccharide, 2.5 sulfate groups per disaccharide)

Table 12 summarizes the K_{d} for ICLX-4 scFv and ICLX-5 scFv.

TABLE 12
K_{d} of ICLX-4 scFv and ICLX-5 scFv (MW 28.08 kDa) for various HSs as
calculated using MotifFinder software

| | | Degree of | K_{d} | |
|---|---|---|---|---|
| Microarray | | polymerization | ICLX-4 | ICLX-5 |
| identifier | GAG name | (dp) | scFv | scFv |
| HS016 | Heparan sulfate | 9 | 3.790 nM | 133.9 nM |
| | (1.8 sulfate groups per | | | |
| | disaccharide) | | | |
| HS021 | Heparan sulfate | 8 | 2.943 nM | 102.9 nM |
| | (2.5 sulfate groups per | | | |
| | disaccharide) | | | |
| HS022 | Heparan sulfate | 8 | 27.15 nM | 116.4 nM |
| | (2.3 sulfate groups per | | | |
| | disaccharide) | | | |
| HS023 | Heparan sulfate | 6 | 2.667 nM | 126.7 nM |
| | (2.7 sulfate groups per | | | |
| | disaccharide) | | | |
| HS024 | Heparan sulfate | 8 | 2.943 nM | 89.28 nM |
| | (2.5 | | | |
| | sulfates/disaccharide) | | | |

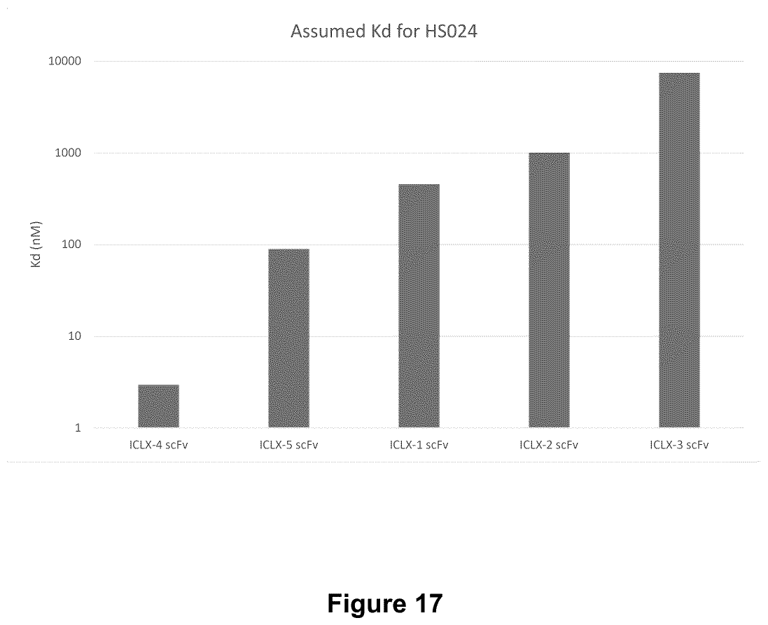

FIG. 17 gives an indication of the assumed K_{d} (in nM) for GAG024 (HS dp8, 2.5 sulfates/disaccharide) of scFv versions of the various antibodies of the invention. The range of K_{d} spans almost 3 orders of magnitude, according to MotifFinder software.

From this chart, the impact on binding to HS024 of various amino acid additions and substitutions in the VHCDR3 (Kabat) of SEQ ID NO:4 of WO97/44461 (as represented by ICLX-3 scFv) is apparent. Each of these changes had a predictable impact on the quality of the CW motifs of the VHCDR3 (Kabat), and the results are confirmed by the assumed K_{d} values, which are given in Table 13 below.

TABLE 13
Summary of the antibodies of the present disclosure and their
respective VHCDR3 amino acid sequence and assumed K_{d}

| Antibody | VHCDR3 (Kabat) | Assumed K_{d} (HS024) |
|---|---|---|
| ICLX-1 scFv | QSLLGDYDHYYGLDA | 454.7 nM |
| ICLX-2 scFv | QSLLGDYDHYYGLDV | 1,004 nM |
| ICLX-3 scFv | DQSLLGDYDHYYGLDV | 7,453 nM |
| ICLX-4 scFv | QSLLGDYDRYYGLDA | 2.943 nM |
| ICLX-5 scFv | QSLLGDYDKYYGLDA | 89.28 nM |

From these results it should be obvious to anyone skilled in the art that the amino acid sequence of all 6 CDR regions of an anti-sulfated GAG antibody, such as ICLX-1, can be modified one amino acid at a time in order to optimize CW motifs, and thus binding to target GAGs.

### Example 6: Testing of Antibody 1HS IgG1 Against GAG and HS Microarrays

Antibody 1HS is disclosed in U.S. Pat. No. 8,551,920 and described as an antibody “ . . . that specifically bind(s) to heparan sulfate and that do(es) not detectably interact with another sulfated carbohydrate or control antigen.” Antibody 1HS was produced as an IgG1 by Creative Biolabs Inc. and sent to Z Biotech, LLC for GAG and HS microarray analysis.

### Method

The glycosaminoglycan (GAG) and heparan sulfate (HS) microarrays were performed as described in Examples 2-5. The following table summarizes the reagents used.

| | | | Other |
|---|---|---|---|
| Reagent | Vendor | Catalog # | information |
| 1HS IgG1 | Creative Biolabs | AGC-0723-CQ1- | [2 mg/mL] |
| | | L5 | |
| Goat Anti-human | Sigma Aldrich | C2571 | — |
| IgG Fc (Cy3) | | | |

The microarray setup and assay methodology are as described in Examples 2-5. Briefly, the microarray was pretreated with Glycan Array Blocking Buffer (GABB) at room temperature for one hour. After blocking, a pre-complexed mixture of 1HS, anti-his tag mAb, and anti-mouse IgG (H+L) AF555 was added to the microarray and incubated at room temperature for 1 hour. The precomplexed mixture initially contained 1HS at 2 μg/mL or 0.4 μg/mL concentration for the GAG microarray and at 10 μg/mL or 2 μg/mL concentration for the HS microarray, anti-his tag mAb at 1:500 dilution, and anti-mouse IgG (H+L) AF555 at 2 μg/mL concentration. This mixture was then diluted by factors of 5 to 6 times. After incubation at room temperature for 1 hour, the microarray was washed and scanned at 532 nm using high laser intensity (1 PMT). Subsequently, the microarrays were analyzed using Mapix (Innopsys, Carbonne, France) microarray analysis software.

The following schematic summarizes the steps performed for the GAG microarray:

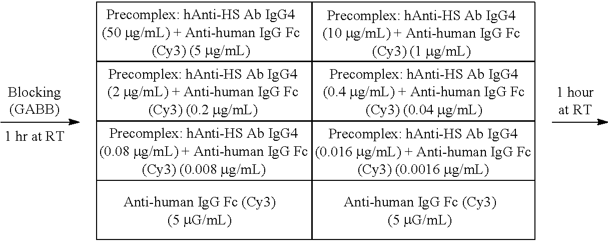

Blocking Buffer: GABB (Glycan Array Blocking Buffer)
Assay buffer GAAB (Glycan Array Assay Buffer)
Working volume: 100 μL/subarray
GAG Array: 16 subarrays, Lot #04132301

The following schematic summarizes the steps performed for the HS microarray:

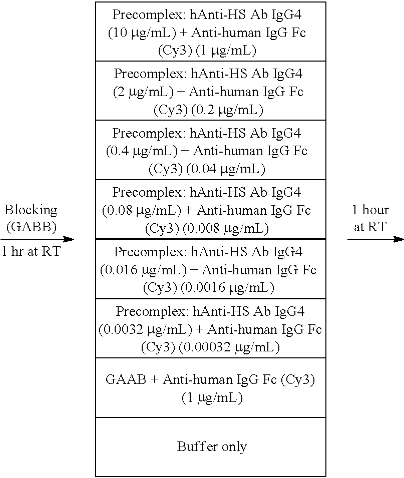

Blocking Buffer: GABB (Glycan Array Blocking Buffer)
Assay buffer GAAB (Glycan Array Assay Buffer)
Working volume: 200 μL/subarray
HS Array: 8 subarrays, Lot #11162101

### Results

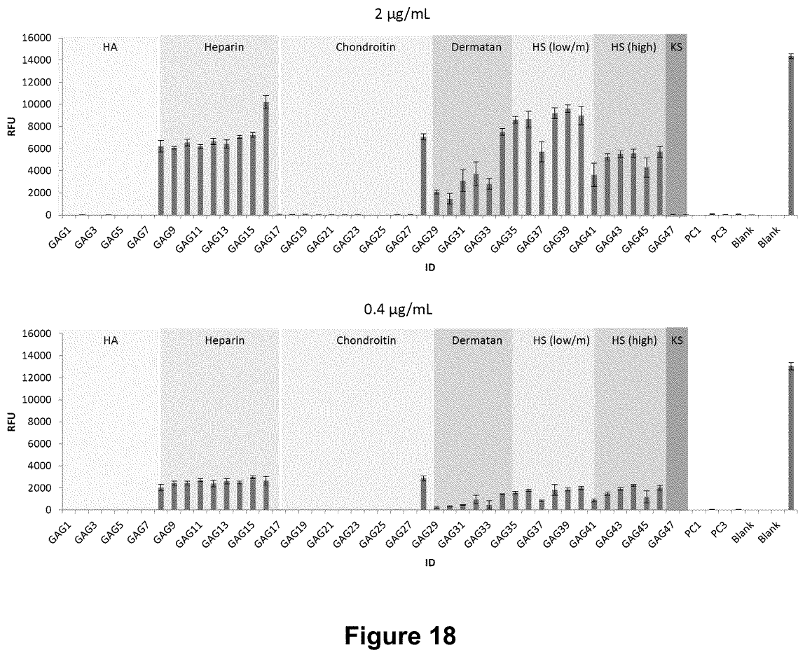

Results for 1 HS binding to the GAG microarray is provided in FIG. 18.

The results indicate that antibody 1HS binds not only to HS, but also to other sulfated GAGs. This was an unanticipated finding.

Once again, CSD dp20 is the only CS that is bound. The pattern of reactivity is almost identical to ICLX-1 IgG4, even though these two antibodies are otherwise unrelated.

In many ways, the pattern of GAG reactivity of ICLX-1 IgG4 resembles ICLX-4 IgG1 more closely than it does ICLX-3 scFv, even though there's only a difference of 2 amino acids in the VHCDR3 (Kabat) of ICLX-1 and ICLX-3.

These results underscore the electrostatic nature of the antibody-GAG interactions for ICLX-1 scFv, ICLX-1 IgG4, ICLX-2 scFv, ICLX-3 scFv, and ICLX-4 IgG1.

The apparent K_{d} of 1HS IgG1 (MW 145.57 kDa) for various GAGs was calculated using MotifFinder software as follows:
9.84 μg/mL or 67.6 nM for GAG38 (Hep I low and intermediate sulfation, dp16)
12.82 μg/mL or 88.07 nM for GAG34 (Dermatan sulfate, dp20)
20.76 μg/mL or 142.6 nM for GAG28 (Chondroitin sulfate D, dp20)
22.51 μg/mL or 154.63 nM for GAG15 (Heparin dp24)
25.07 μg/mL or 172.2 nM for GAG46 (Hep III high sulfation, dp20)

The results are summarized in Table 16 below.

TABLE 16
K_{d} of 1HS IgG1 (MW 145.57 kDa) for various GAGs as calculated
using MotifFinder software

| | | Degree of | |
|---|---|---|---|
| Microarray | | polymerization | |
| identifier | GAG name | (dp) | K_{d} |
| GAG38 | Heparan sulfate I | 16 | 67.6 nM |
| | (low and intermediate sulfation) | | |
| GAG34 | Dermatan sulfate | 20 | 88.07 nM |
| GAG28 | Chondroitin sulfate D | 20 | 142.6 nM |
| GAG15 | Heparin (Hep) | 24 | 154.63 nM |
| GAG46 | Heparan sulfate III | 20 | 172.2 nM |
| | (high sulfation) | | |

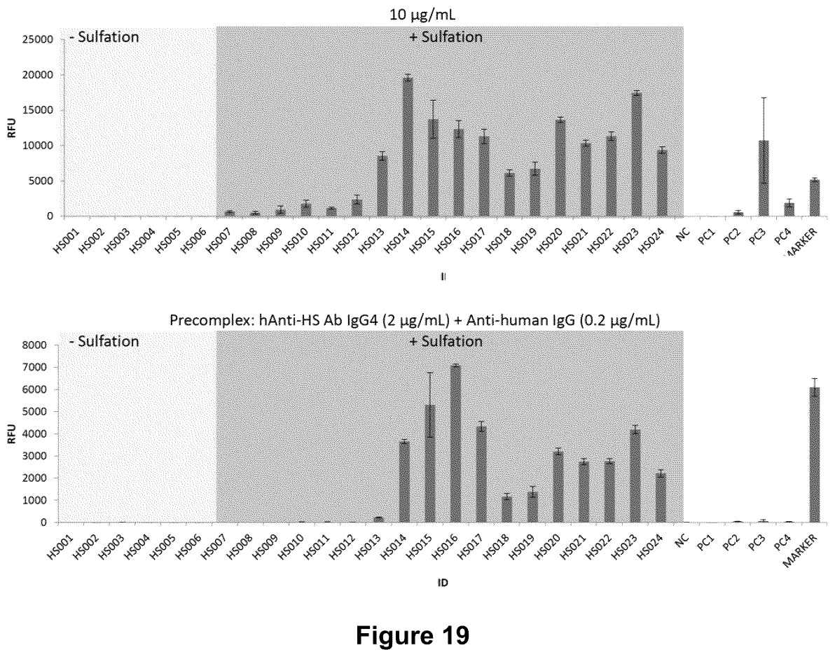

Results for 1HS binding to the GAG microarray is provided in FIG. 19. The apparent affinity of antibody 1HS for GAG38 (Hep I low and intermediate sulfation, dp16) was the highest of any GAG species tested. Antibody 1HS IgG1 may thus be regarded as primarily an anti-HS antibody that cross-reacts with other sulfated GAG species. Reactivity of 1HS with Hep, CSD, and DS represents novel uses of this antibody.

The apparent Kd of 1HS IgG1 (MW 145.57 kDa) for various HSs was calculated using MotifFinder software as follows:
1.846 μg/mL or 12.71 nM for HSO16 (nonasaccharide, 1.8 sulfate groups per disaccharide)
69.11 μg/mL or 474.8 nM for HSO21 (octasaccharide, 2.5 sulfate groups per disaccharide)
53.61 μg/mL or 386.8 nM for HS022 (octasaccharide, 2.3 sulfate groups per disaccharide)
26.97 μg/mL or 185.3 nM for HS023 (hexasaccharide, 2.7 sulfate groups per disaccharide)
68.78 μg/mL or 472.5 nM for HS024 (octasaccharide, 2.5 sulfate groups per disaccharide)

The results are summarized in Table 17 below.

TABLE 17
Kd of 1HS IgG1 (MW 145.57 kDa) for various HSs as calculated
using MotifFinder software

| | | Degree of | |
|---|---|---|---|
| Microarray | | polymerization | |
| identifier | GAG name | (dp) | K_{d} |
| HS016 | Heparan sulfate | 9 | 12.71 nM |
| | (1.8 sulfates/disaccharide) | | |
| HS021 | Heparan sulfate | 8 | 474.8 nM |
| | (2.5 sulfate groups per | | |
| | disaccharide) | | |
| HS022 | Heparan sulfate | 8 | 386.8 nM |
| | (2.3 sulfate groups per | | |
| | disaccharide) | | |
| HS023 | Heparan sulfate | 6 | 185.3 nM |
| | (2.7 sulfate groups per | | |
| | disaccharide) | | |
| HS024 | Heparan sulfate | 8 | 472.5 nM |
| | (2.5 sulfates/disaccharide) | | |

Taken together, a summary of the different K_{d} for each antibody and the different GAGS is presented in Table 18 below.

| | K_{d} for antibody and specific GAG | | | | | | | | | | | | | | | | |
|---|---|---|---|---|---|---|---|---|---|---|---|---|---|---|---|---|---|
| | ICLX-1 | | | | | | ICLX-3 scFv | | | | | | | | | | |
| | | | | | ICLX-2 | | | | pH K_{d} (μM) | | | ICLX-4 | | ICLX-5 | | 1HS | |
| GAG # identifier | scFv | | IgG4 | | scFv | | | | 7.4 | 6.8 | 6.0 | scFv | | scFv | | IgG1 | |
| GAG15 (Hep) | 125 | nM | 92.61 | nM | 235 | nM | 1.860 | nM | | | | 35 | nM | 173 | nM | 154.63 | nM |
| GAG28 (CSD) | 199 | nM | 121.3 | nM | 252 | nM | 5.65 | μM | | | | 45.9 | nM | 186 | nM | 142.6 | nM |
| GAG46 (HSIII) | 336 | nM | 237.11 | nM | 1.24 | μM | 35.0 | μM | | | | 55.5 | nM | 329 | nM | 172.2 | nM |
| GAG38 (HSI) | 385 | nM | 296.5 | nM | 17.3 | μM | | | | | | 57.7 | nM | 277 | nM | 67.6 | nM |
| GAG34 (DS) | 431 | nM | 546.3 | | 18.9 | μM | | | | | | 222 | nM | 269 | nM | 88.07 | nM |
| HS016 (1.8 S/d) | 399.2 | nM | 44.67 | nM | 580.2 | nM | 7.420 | μM | | | | 3.790 | nM | 133.9 | nM | 12.71 | nM |
| HS020 (2.3 S/d) | | | | | | | | | 1.417 | 0.4453 | 0.3631 | | | | | | |
| HS021 (2.5 S/d) | 529.6 | nM | 70.68 | nM | 1.056 | μM | 9.139 | μM | 1.333 | 6.858 | 0.4141 | 2.943 | nM | 102.9 | nM | 474.8 | nM |
| HS022 (2.3 S/d) | 474.4 | nM | 82.26 | nM | 9.176 | μM | 3.762 | μM | 14.71 | 1.836 | 0.3325 | 27.15 | nM | 116.4 | nM | 386.8 | nM |
| HS023 (2.7 S/d) | 776.0 | nM | 107.1 | nM | 2.238 | μM | 19.75 | μM | 12.44 | 15.34 | 0.7538 | 2.667 | nM | 126.7 | nM | 185.3 | nM |
| HS024 (2.5 S/d) | 454.8 | nM | 85.87 | nM | 1.004 | μM | 7.453 | μM | 9.901 | 0.9493 | 0.4052 | 2.943 | nM | 89.28 | nM | 472.5 | nM |

### Example 7: Effect of Anti-GAG Antibodies on Cancer Cell Viability In Vitro

The effect of ICLX-1 IgG1 on cancer cell viability was investigated in a variety of cell lines using the CellTiter-Glo® assay. The CellTiter-Glo® Luminescent Cell Viability Assay offers several advantages, making it a popular choice for measuring cell viability in various research and screening applications. The assay quantifies ATP, a marker of metabolically active cells, making it a reliable indicator of cell viability. The amount of ATP is directly proportional to the number of viable cells, facilitating accurate assessment of cell health and viability. The assay provides a broad linear range, allowing accurate quantification of viable cells over a wide range of cell concentrations.

### RPMI 8226

RPMI 8226 cells are B lymphocytes isolated in 1966 from the peripheral blood of a 61-year-old, male, plasmacytoma patient. The multiple myeloma (MM) cell line, RPMI 8226, like other MM cell lines, preferentially expresses syndecan-1. According to The Human Protein Atlas (www.proteinatlas.org), transcript levels for SDC1 are 159.2 nTPM in RPMI 8226. The effect of the ICLX-1 IgG1 on cell proliferation was first tested in RPMI 8226 cells.

### Method

RPMI 8226 cells were seeded in a 12 well culture dish at an initial concentration of 50,000 cells per well. The cells were cultured in complete Gibco Minimum Essential Media (MEM) supplemented with 10% fetal bovine serum. At two days in culture, ICLX-1 IgG1 was added to the cells at different concentrations. Two or four days later, the cell viability assay CellTiter-Glo® Luminescent (Cat #G7570) was performed according to the manufacturer's (Promega) protocol. Briefly, the CellTiter-Glo® Luminescent Cell Viability Assay is a homogeneous method of determining the number of viable cells in culture based on quantitation of the ATP present, an indicator of metabolically active cells. The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells/well in a 384-well format in 10 minutes after adding reagent and mixing. The homogeneous “add-mix-measure” format results in cell lysis and generation of a luminescent signal proportional to the amount of ATP present. The amount of ATP is directly proportional to the number of cells present in culture. The CellTiter-Glo® Assay generates a “glow-type” luminescent signal, which has a half-life generally greater than five hours, depending on cell type and medium used.

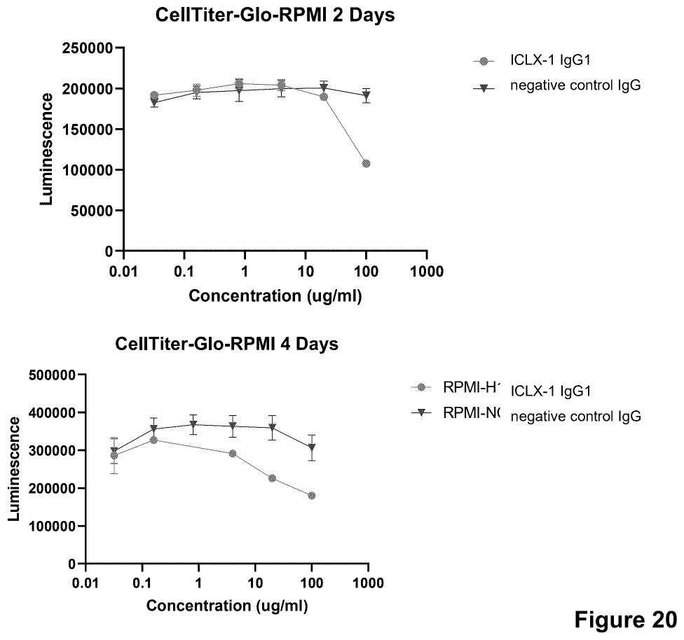

FIG. 20 shows a reduction in cell viability in the presence of the ICLX-1 IgG1 both after 2 days and 4 days of the antibody treatment.

### A375

A375 cells, a human melanoma cell line initiated through explant culture of a solid tumor from a 54-year-old female, were then tested.

### Method

A375 cells were seeded in a 12 well culture dish at an initial concentration of 50,000 cells per well. The cells were cultured in complete Gibco Minimum Essential Media (MEM) supplemented with 10% fetal bovine serum. At two days in culture, ICLX-1 IgG1 was added to the cells at different concentrations. Two or four days later, the cell viability assay CellTiter-Glo® Luminescent (Cat #G7570) was performed according to the manufacturer's (Promega) protocol, as described above.

### Results

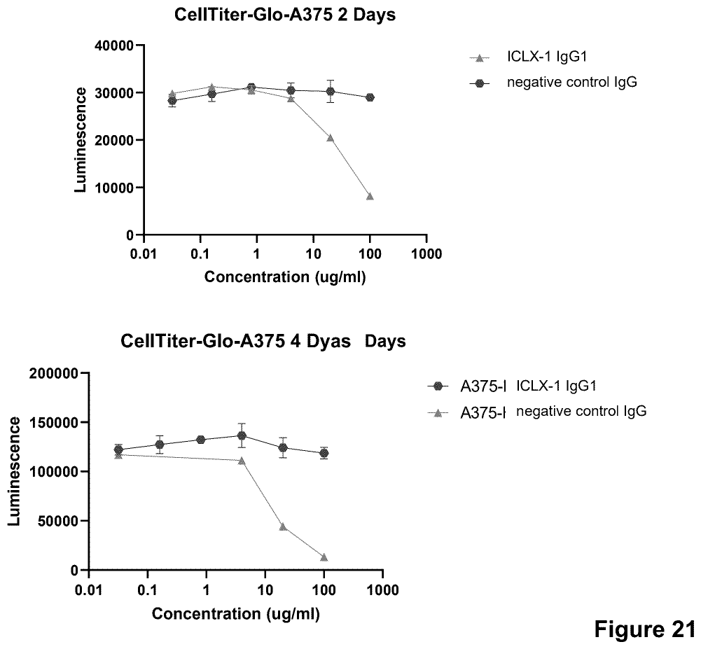

The results depicted in FIG. 21 for A375 were most impressive. After 4 days in culture, there was an almost 100% decrease in cell viability at the highest concentration of ICLX-1 IgG1. These results also demonstrate that neither complement nor effector cells are required for loss of viability.

### BxPC3

BxPC3 cells were then tested. BxPC3 is a cell line exhibiting epithelial morphology that was isolated from the pancreas tissue of a 61-year-old, female patient with adenocarcinoma.

### Method

BxPC3 cells were seeded in a 12 well culture dish at an initial concentration of 50,000 cells per well. The cells were cultured in complete Gibco Minimum Essential Media (MEM) supplemented with 10% fetal bovine serum. At two days in culture, ICLX-1 IgG1 was added to the cells at different concentrations. Two or four days later, the cell viability assay CellTiter-Glo® Luminescent (Cat #G7570) was performed according to the manufacturer's (Promega) protocol, as described above.

### Results

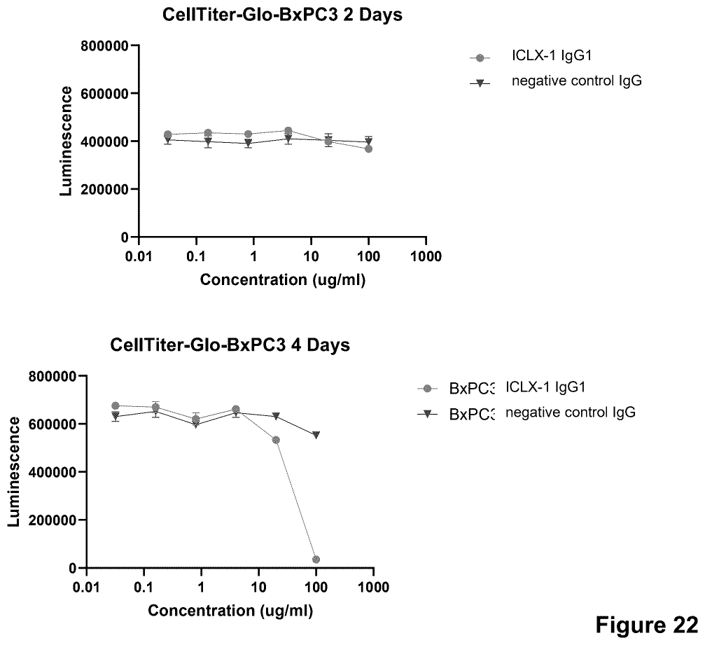

The results depicted in FIG. 22 for were also quite striking. After 4 days in culture, an almost 100% decrease in cell viability was observed with the highest dose of ICLX-1 IgG1. These results further demonstrate that neither complement nor effector cells are required for cell killing.

### Daudi

The Daudi cell line is composed of B lymphoblasts isolated from the peripheral blood of a Black, 16-year-old, male, Burkitt's Lymphoma patient in 1967.

### Method

Daudi cells were seeded in a 12 well culture dish at an initial concentration of 50,000 cells per well. The cells were cultured in complete Gibco Minimum Essential Media (MEM) supplemented with 10% fetal bovine serum. At two days in culture, ICLX-1 IgG1 was added to the cells at different concentrations. Two or four days later, the cell viability assay CellTiter-Glo® Luminescent (Cat #G7570) was performed according to the manufacturer's (Promega) protocol, as described above.

### Results

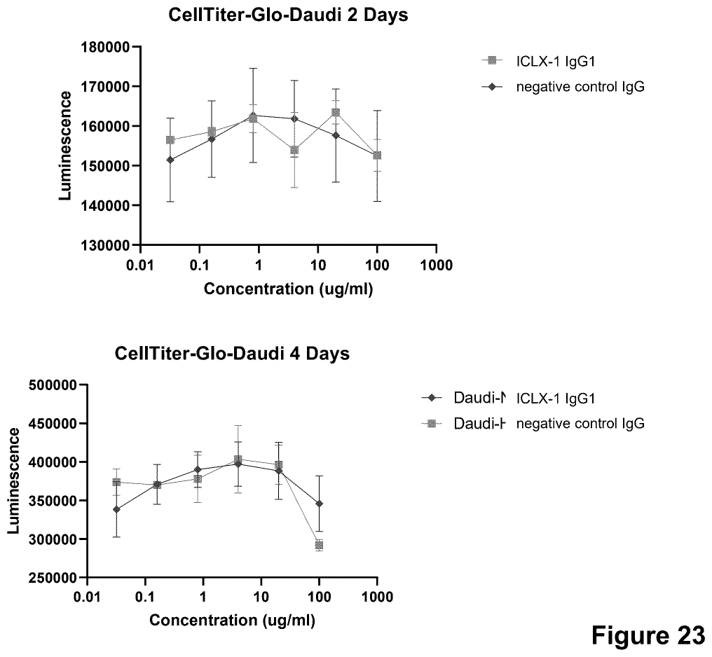

The results depicted in FIG. 23 show a negligible effect of ICLX-1 IgG1 on Daudi cell viability compared with the IgG-matched negative control, even after 4 days of incubation at the highest concentration (100 μg/mL). This is likely because Daudi cells have a very low level of expression of HSPGs (GPC1=3.7; GPC2=5.2 nTPM).

### A375

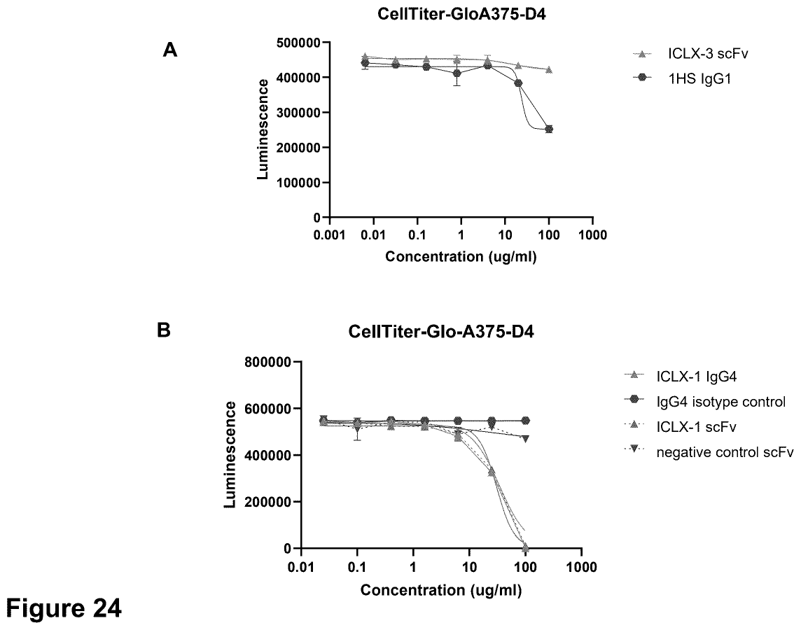

Other antibodies were tested on A375 cells as described above. FIG. 24 summarizes the results. FIG. 24A shows that ICLX-3 scFv (mislabeled as ICLX-4 scFv) does not influence the viability of A375 cells. On the other hand, antibody 1HS IgG1 (commercially sourced from Creative Biolabs Inc.) had a negative impact on cell viability at the highest concentration (100 μg/mL). In contrast with ICLX-3 scFv, FIG. 24B shows that ICLX-1 IgG4 and ICLX-1 scFv both had a dramatic effect on cell viability, highlighting the potential for these antibodies as anticancer agents. Moreover, these results show the ability of the antibodies of the present invention to kill cancer cells.

### Example 8: Inhibition of HGF Binding to HepG2 Cells by an Anti-HS Antibody

Hepatocyte Growth Factor binding to HepG2 cells was chosen as a model to illustrate the effect of an anti-GAG antibody (in this case, ICLX-1 scFv) on the binding of a growth factor to its canonical receptor. As mentioned previously, negatively charged glycosaminoglycans on membrane-tethered proteoglycans act as co-receptors for growth factor binding to cancer cells by interacting with positively charged amino acids on the growth factor. An anti-GAG antibody, such as ICLX-1 scFv, will thus sterically interfere with this binding process by binding to the GAG sidechains of cell surface proteoglycans with greater affinity than the growth factor/GAG interaction.

HepG2 hepatocellular carcinoma is a cell line exhibiting epithelial-like morphology, and was isolated from a 15-year-old, white male with liver cancer. The canonical receptor for HGF is the c-MET proto-oncogene. HGF normally stimulates HepG2 growth (MET=27.3 nTPM) but has minimal effect on the MET-negative multiple myeloma cell line, RPMI 8226 (MET=0.3 nTPM).

### Method

HepG2 cells were seeded in a 96 well culture dish at an initial concentration of 10,000 cells per well. The cells were cultured in complete Gibco Minimum Essential Media (MEM) in the absence of serum or supplemented with 10% fetal bovine serum. At two days in culture, ICLX-1 scFv was added to the cells at about 50 μg/mL with or without HGF (200 ng/mL). Cell viability was assessed at 1 day, 3 days and 5 days following treatment. Five replicates were measured for each time point. The cell viability assay CellTiter-Glo® Luminescent (Cat #G7570) was performed according to the manufacturer's (Promega) protocol, as described above. Five replicates were measured for each time point.

### Results

### HepG2

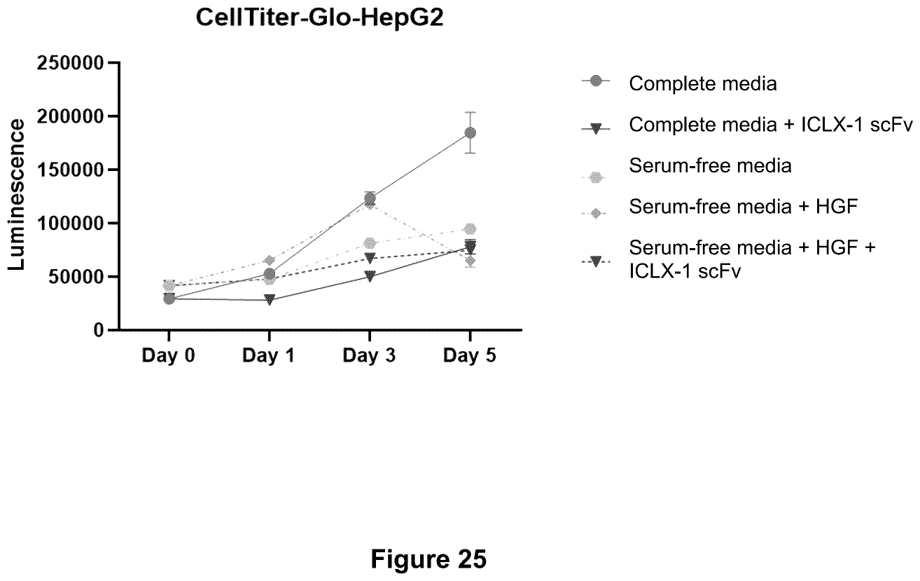

FIG. 25 shows that ICLX-1 scFv effectively blocked the effect of HGF on HepG2 in serum free media. The absence of FBS in the media leads to a reduction in cell viability (yellow line, octagons). In contrast, the addition of HGF into the serum-free media increases cell viability and reconstitutes the cell growth to what was observed in the complete media, but only until day 3. The amount of HGF added does not seem to suffice in compensating for the lack of resources in the serum free media as the number of cells keeps increasing (green line, squares). Interestingly, the presence of ICLX-1 scFv in the complete media (blue triangles) reduced cell viability and inhibited cell growth even more than the absence of the FBS. Moreover, the presence of ICLX-1 scFv in the serum-free media with HGF eliminated the recovery of cell viability and growth rate observed in the presence of HGF at day 3, even if temporary (purple triangles).

### RPMI 8226

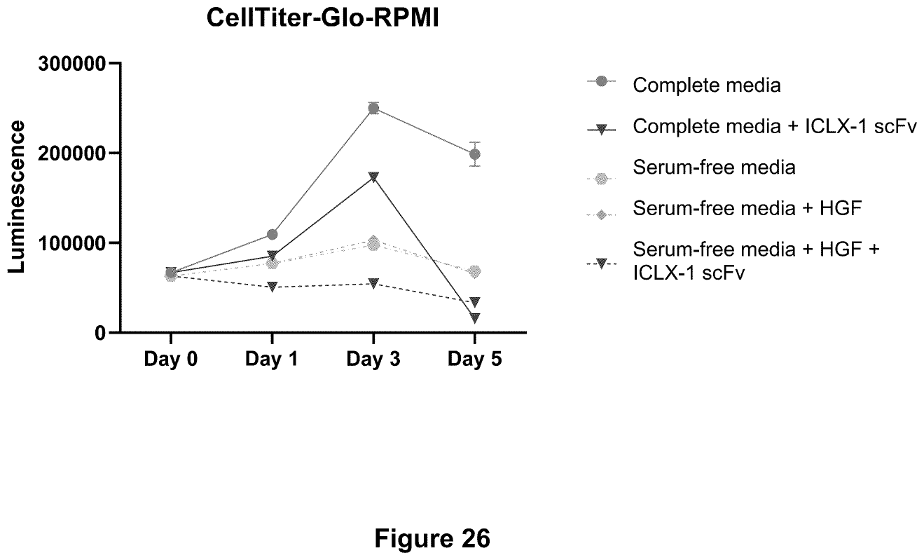

As expected, neither HGF nor ICLX-1 scFv had any effect on RPMI 8226 cell viability and growth in serum free media, as shown in FIG. 26. These cells followed an exponential cell growth only until Day 3 when cultured in complete media. Cell viability was reduced in the absence of FBS in the media (yellow octagons), as expected. Similarly to HepG2 cells, the presence of ICLX-1 scFv (blue triangles) in the complete media reduced cell viability and inhibited cell growth, albeit to lesser extent than in the absence of the FBS. It appears that RPMI 8226 is more sensitive to the absence of FBS than it is to the presence of ICLX-1 scFv. The addition of HGF into the serum-free media (green squares) failed to reconstitute the cell growth to what was observed in the complete media (red circles). Following the addition of ICLX-1 scFv into the serum-free media with HGF (purple triangles), a further decreased in luminescence was observed, possibly a proxy for further accelerated cell death.

HGF interacts with the sulfated GAGs HS and DS (Lyon, et al., 1998; Lyon et al., 2002). Yamaguchi, et al. (2006) showed that HGF has preferential binding to iduronic acid-rich fragments of DS oligosaccharides that are greater than 8-mer in length. ICLX-1 scFv specifically binds to the latter (see Example 2). The receptor for HGF is encoded by the gene MET. MET mRNA levels in HepG2 are 27.3 nTPM, and only 0.3 nTPM in RPMI 8226. Taken together, the results described herein are consistent given the near absence of a receptor for HGF on RPMI 8226.

### Example 9: Cell Death Analysis of RPMI 8226 Cells Treated with ICLX-1 scFv

Whereas the CellTiter-Glo® assay provides a good indicator of cell viability, it is not considered as definitive. Only flow cytometry (FCM) with propidium iodide (PI) staining directly measures cell membrane integrity, thus making it a definitive indicator of cell death. PI penetrates only dead cells with compromised membranes, providing a clear distinction between live and dead cells. The effect of ICLX-1 scFv on RPMI 8226 cells was therefore assessed with FCM and PI staining.

### Method

The effect of ICLX-1 scFv was evaluated based on co-incubation of RPMI 8226 cells with a concentration gradient of ICLX-1 scFv followed by cell death analysis by flow cytometry.

RPMI 8226 is a multiple myeloma (plasmacytoma) cell line sourced from the ATCC (CCI-155™), isolated in 1966 from the peripheral blood of a 61-year-old male. The cells were grown in complete RPMI medium (RPMI-1640+10% FBS) at 37° C. and 5% CO₂ and maintained at a concentration of between 5×10⁵ and 2×10⁶ cells/mL and passaged every 2-3 days.

Culturing conditions: 10,000 cells in 100 uL 10% FBS/RPMI 1640 media, cultured at 37° C., 5% CO₂ for 4 days.

Annexin/7-AAD Apoptosis kit (Sigma, cat #CBA059) was used for flow cytometry analysis to define dead, apoptotic and live cells. Briefly, the following steps were performed:
Spin down the plate at 500 g for 5 minutes.
Wash cells twice with 200 μL cold DPBS.
Resuspend into 50 μL of Annexin V Binding Buffer containing 5 μL/per sample of APC Annexin V. Mix well.
Add 50 μL of Annexin V Binding Buffer containing 5 μL/per sample of 7-AAD. Mix well.
Incubate for 15 min at room temperature in the dark.
Add 400 μL of Annexin V Binding Buffer to each well by transferring samples into 96 deep well plate.
Analyze by flow cytometry with proper machine settings.

### Results

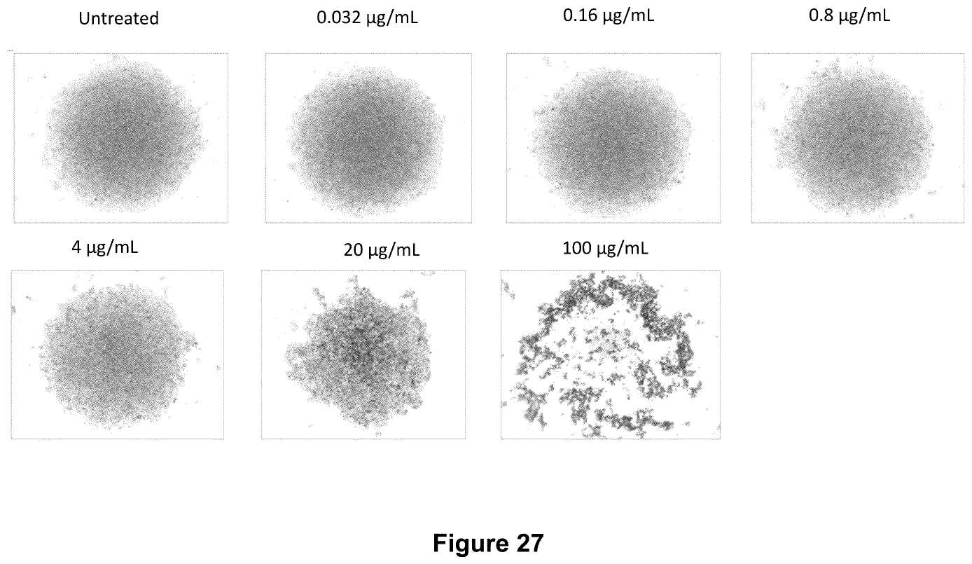

As shown in FIG. 27, ICLX-1 scFv appears to be toxic to RPMI 8226 cells after 4 days of treatment at a concentration of 20 μg/mL and higher. The majority of RPMI 8226 cells appear to be dead when treated with ICLX-1 scFv at a concentration of 100 μg/mL. Morphologically, the cells appear to undergo necrotic death following ICLX-1 scFv treatment. These qualitative observations were confirmed as barely any annexin V single positive cells (apoptotic) could be detected, indicating that a majority of cells were dying by necrotic cell death, and not via apoptosis.

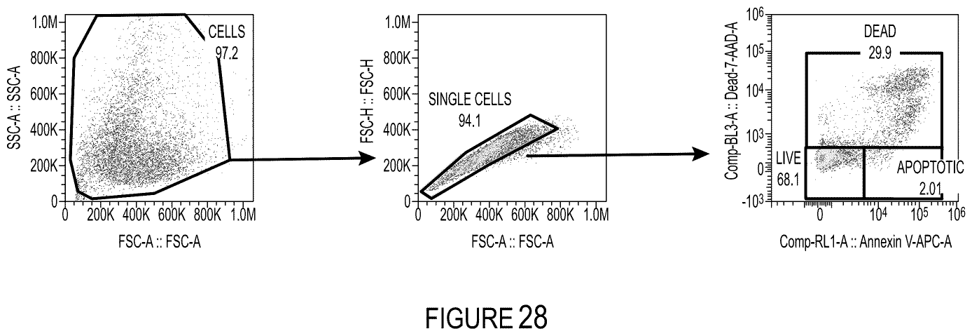

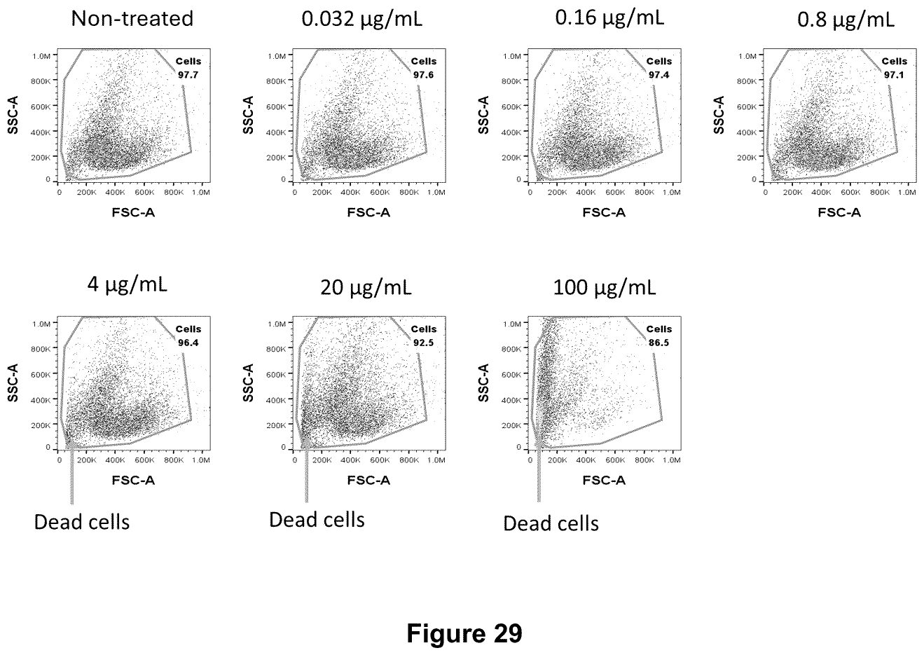

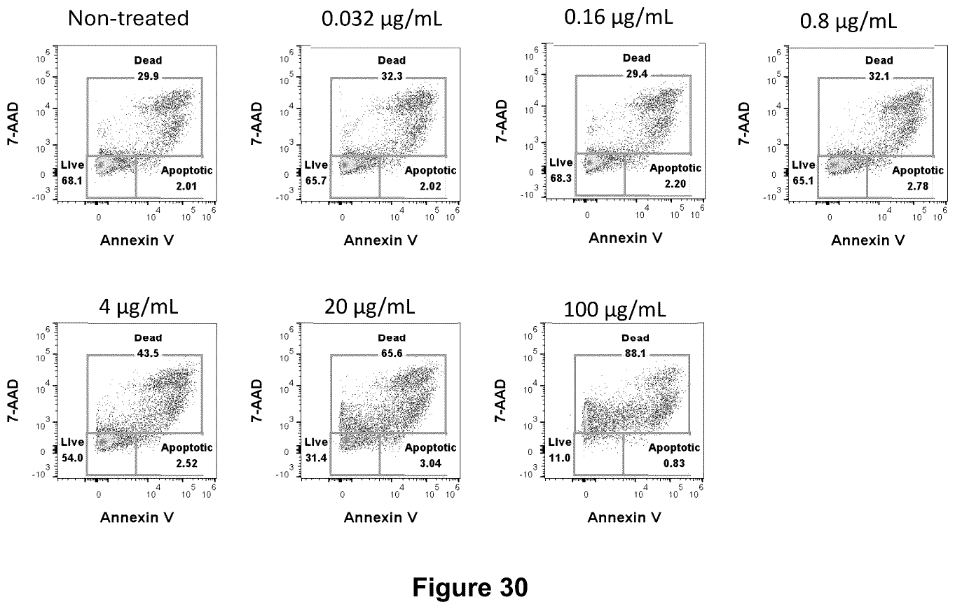

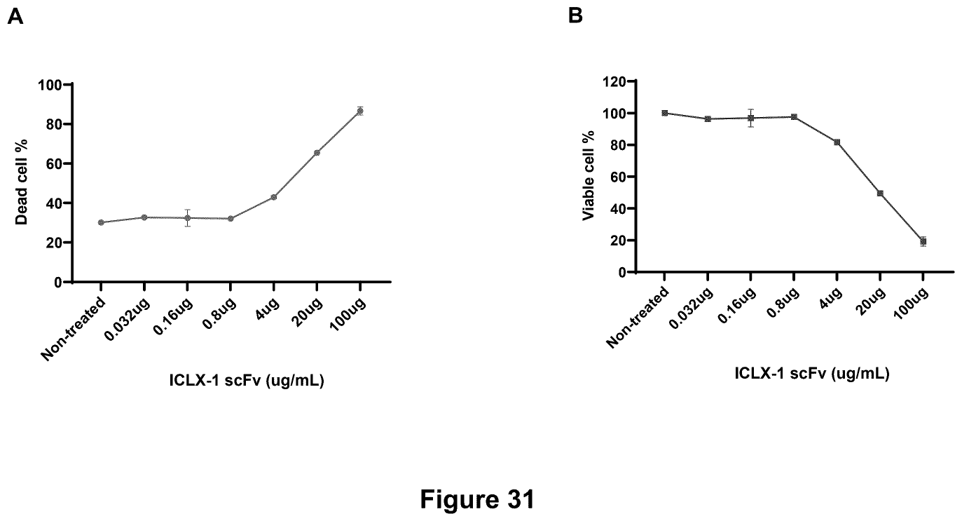

The flow cytometry experiments depicted in FIGS. 28-30 and the quantification thereof depicted in FIG. 31 confirmed these qualitative observations made from the images of FIG. 27. Indeed, the percentage of dead cells reached around 90% when treated with 100 μg/mL of ICLX-1 scFv. This was accompanied by the majority of cells losing their normal forward scatter (FSC)/side scatter (SSC) pattern at this concentration (FIG. 30-32).

The percentage of dead cells increased rapidly from 0.8 μg/mL, reaching about 90% when treated with 100 μg/mL. Indeed, the viable cell percentages dropped quickly from 0.8 μg/mL after normalizing against non-treated group (FIG. 31).

Taken together, FIGS. 27-31 demonstrate an efficient cell killing mechanism by ICLX-1 scFv, even at low concentrations. Thus, ICLX-1 scFv is a good candidate for killing cancer cells in vitro or ex vivo, and may also be a good candidate as a cancer therapy for killing cancer cells in vivo given that a related antibody (NovoMAb-G2 scFv) was previously shown to be effective in tumor xenograft models.

### Example 10: ICLX-1 scFv Testing of Solid Tumor 3D Spheroids

Multiple myeloma grows in vitro as a suspension culture and does not pose many challenges insofar as antibody access to individual cells is concerned. More challenging, however, are the solid tumors, such as pancreatic and lung carcinoma. The latter grow as clumps of cells, many of which are located at a distance from blood vessels in vivo. Since ICLX-1 scFv showed great potential at killing a multiple myeloma (MM) cell line (i.e., RPMI 8226 cells) in the previous Example, this antibody, and its IgG4 counterpart, were chosen for testing on solid tumor 3D spheroids. The 3D tumor spheroid technique is a good representation of the poorly oxygenated, mildly acidic, tumor microenvironment (TME) of a solid tumor. Additionally, 3D tumor spheroids are a good way of comparing the tumor penetrating abilities of a scFv versus an IgG version of a given antibody (e.g., ICLX-1 scFv vs. ICLX-1 IgG4).

MIA PaCa-2 is a human pancreatic cancer cell line used extensively in pancreatic cancer research and therapy development. The cells express CK5.6, AE1/AE3, E-cadherin, vimentin, chromogranin A, synaptophysin, SSTR2, and NTR1, but do not express CD56. They have a round, epithelial morphology, and are adherent in cell culture. They are widely used as a model of pancreatic cancer, and studies of MIA PaCa-2 physiology have helped clarify the mechanisms of carcinogenesis in pancreatic cancer.

A549 cells are adenocarcinomic human alveolar basal epithelial cells widely used as a model of lung cancer. They are squamous and responsible for the diffusion of substances, such as water and electrolytes, across alveoli. A549 cells are widely used as a type II pulmonary epithelial cell model for drug metabolism. Although A549 is a cancer cell line, it has also been studied for its response to tuberculosis, specifically the production of chemokines as it is induced by the invading bacteria.

PANC-1 is a human pancreatic cancer cell line isolated from a pancreatic carcinoma of ductal cell origin. They are known for their epithelial morphology and are adherent in cell culture flasks. PANC-1 cells have been used to study the role of keratin reorganization during the migration of cancer cells, along with calcium-mediated actin reset in response to physiological changes.

SK-N-FI is a neuroblastoma cell line derived from a bone marrow metastasis from a 11-year-old Caucasian male with poorly differentiated embryonal neuroblastoma. The cells exhibit high MDR1 expression.

### Method

Four different 3D tumor spheroid cell lines were incubated with various concentrations of ICLX-1 (scFv or IgG4), ICLX-3 (scFv or IgG4), or a positive control compound (staurosporine). The main biological activity of staurosporine is the inhibition of protein kinases through the prevention of ATP binding to the kinase. This is achieved through the stronger affinity of staurosporine to the ATP-binding site on the kinase. Staurosporine is widely used to induce apoptosis although the mechanism is not well understood.

Staurosporine was purchased from Sigma-Aldrich (cat #S5921) and the CellTiter-Glo® 3D cell viability assay reagent was purchased from Promega (cat #G9681). MIA PaCa-2, A549, PANC-1 and SK-N-FI cells were purchased from American Type Culture Collection (Manassas, VA).

Cell culture media are listed in the table below. All media were supplemented with 100 μg/ml of penicillin and 100 μg/ml of streptomycin unless otherwise stated. Cultures were maintained at 37° C. in a humidified atmosphere of 5% CO₂ and 95% air.

TABLE 19
Summary of conditions used for growing each cell line

| Cell Line | Media |
|---|---|
| MIA PaCa-2 | DMEM + 2.5% horse serum + 10% FBS |
| A549 | F-12K + 10% FBS |
| PANC-1 | DMEM + 10% FBS |
| SK-N-FI | DMEM + 10% FBS |

ICLX-1 scFv, ICLX-1 IgG4, and staurosporine were tested in 10-dose IC₅₀ mode in quadruplicate with 3-fold serial dilution starting at 50 μg/mL (ICLX-1 scFv) and 10 μM (staurosporine). The following step were performed:
32 μL (ICLX-1) or 36 μL (for staurosporine) of culture medium containing 2,000 of MIA PaCa-2, A549 or PANC-1 cells were seeded to each of the wells of Corning spheroid 384-well microplate and incubated over night at 37° C., 5% CO₂.
After 1 day in culture, the test antibody, ICLX-1, or staurosporine, were diluted in culture medium in 10-dose with 3-fold serial dilution in a 96-well dilution plate. 8 μL of ICLX-1 or 4 μL of staurosporine, were added to the respective cell wells of the Corning spheroid 384-well microplate. The final concentration of the test antibody started at 50 μg/mL, 10-dose in quadruplicate with 3-fold dilution. The final concentration of staurosporine started at 10 μM, 10-dose in quadruplicate with 3-fold dilution.
The cells were incubated with the test antibody or staurosporine at 37° C., 5% CO₂ for 6 days.
Equal volume (40 μl) of Cell Titer-Glo® 3D Reagent was added to each well.
The contents were mixed on an orbital shaker for 5 min and incubated at room temperature for an additional 25 min to stabilize luminescent signal.
Luminescence was recorded by Envision 2104 Multilabel Reader (PerkinElmer, Santa Clara, CA). The number of viable cells in culture was determined based on quantitation of the ATP present in each culture well.
A curve of the concentration of each tested compound where the percent inhibition is equal to 50% (IC₅₀) was plotted for each compound and cell line, and IC₅₀ values were calculated using the GraphPad Prism program based on a sigmoidal dose-response equation.

### Results

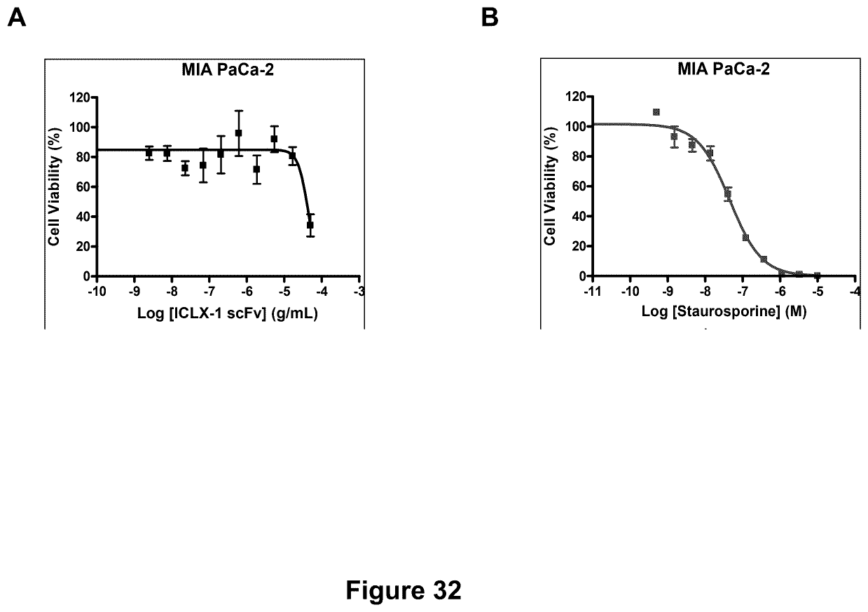

Both ICLX-1 scFv (FIG. 32) and ICLX-1 IgG4 (data not shown) were effective at killing MIA PaCa-2 3D spheroids after 6 days of treatment although ICLX-1 scFv was generally more effective at killing cells than ICLX-1 IgG4. Interestingly, ICLX-3 (scFv and IgG4) was not effective at killing the MIA PaCa-2 cell line (data not shown).

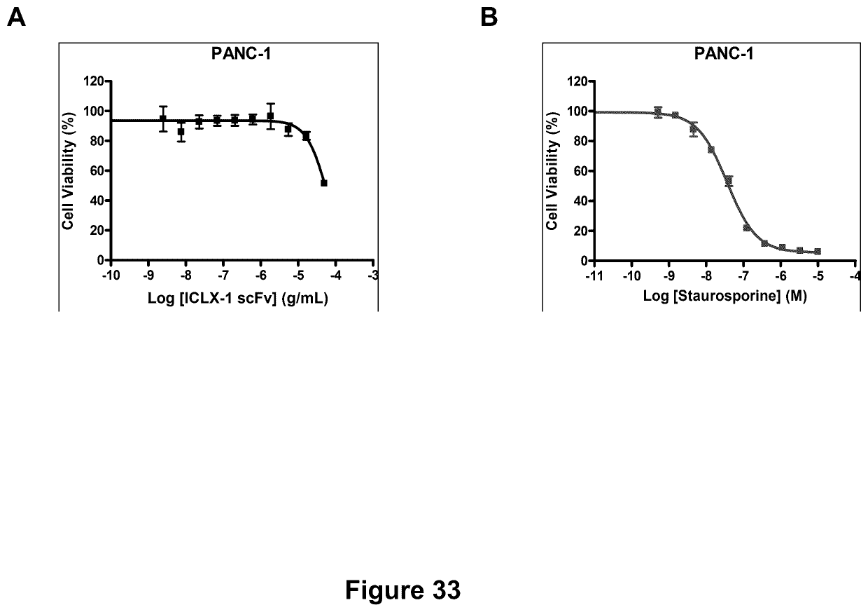

ICLX-1 scFv was also mildly effective at killing PANC-1 3D spheroids after 6 days of treatment (FIG. 33), whereas ICLX-1 IgG4, ICLX-3 (scFv and IgG4) did not appear to be toxic to PANC-1 cells at the tested concentrations (data not shown).

The only antibody that was effective at killing lung carcinoma 3D spheroids (A549 cell line) was ICLX-1 scFv as ICLX-1 IgG4 was ineffective at killing A549 cells after 6 days of treatment at the tested concentrations. ICLX-3 scFv and ICLX3 IgG4 were also ineffective at killing A549 cells after 6 days of treatment at the tested concentrations.

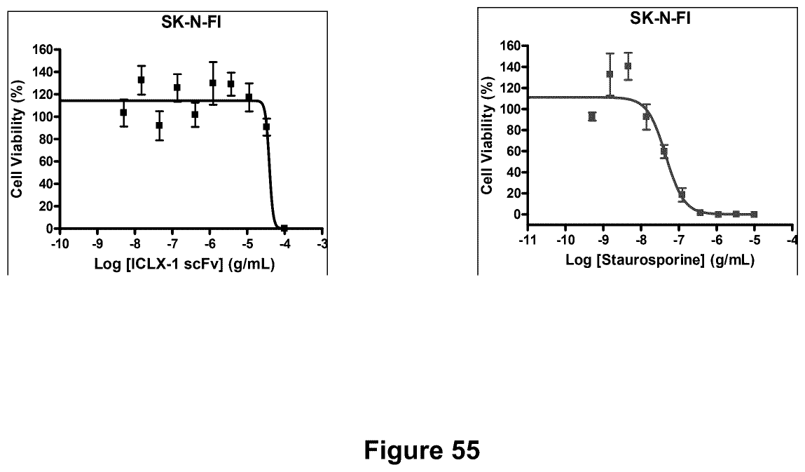

The only antibody that was tested on the SK-N-FI neuroblastoma 3D spheroids was ICLX-1 scFv, which was able to kill 100% of the cells at the highest concentration tested (100 μg/mL) (FIG. 55).

Table 20 below summarizes the results for all tested antibodies and cell lines.

TABLE 20
Summary of the concentration of each tested compound where the percent
inhibition is equal to 50% (IC₅₀)
CellTiter-Glo ® 3D cell viability assay

| | Cell Line | | | |
|---|---|---|---|---|
| | IC₅₀ value (g/mL) | | | |
| Compound ID | A549 | MIA PaCa-2 | PANC-1 | SK-N-FI |
| ICLX-1 scFv | 8.14 × 10⁻⁶ | 4.42 × 10⁻⁵ | 5.73 × 10⁻⁵ | 3.88 × 10⁻⁵ |
| ICLX-1 IgG4 | >5 × 10⁻⁵ | 1.27 × 10⁻⁵ | >5 × 10⁻⁵ | |
| ICLX-3 scFv | >5 × 10⁻⁵ | >5 × 10⁻⁵ | >5 × 10⁻⁵ | |
| ICLX-3 IgG4 | >5 × 10⁻⁵ | >5 × 10⁻⁵ | >5 × 10⁻⁵ | |
| staurosporine (M) | 5.02 × 10⁻⁹ | 4.49 × 10⁻⁸ | 3.62 × 10⁻⁸ | 4.49 × 10⁻⁸ |

Taken together, ICLX-1 scFv appears to be a good candidate as an anti-cancer agent as the 3D tumor spheroid experiments show that ICLX-1 scFv is efficient at killing cancer cells in an environment that mimics the in vivo tumor microenvironment.

### Example 11: GAG, GSL and HS Microarrays Testing of AO4B08 scFv, 1HS scFv and 3HS scFv Antibodies

The flow cytometry (FCM) results obtained with ICLX-1 scFv and RPMI 8226 were very encouraging, however, these could possibly be the result of ICLX-1 scFv's unique CDR sequences. Three other polyspecific anti-GAG antibodies with CDR sequences unrelated to ICLX-1 scFv were therefore chosen for a subsequent FCM experiment. But first, all 3 antibodies had to prepared as scFvs in order to compare them properly to ICLX-1 scFv and not introduce any variables related to avidity (an IgG has twice the avidity of a scFv, and 1 HS was originally supplied as an IgG1). Next, all 3 antibodies had to be studied in GAG, HS, and GSL microarrays in order to ensure that none of them bound to NeuGcGM3 because the polyspecific anti-GAG antibody, chP3R99, is also anti-NeuGcGM3 and its anti-cancer properties are allegedly mediated through its anti-NeuGcGM3 binding.

This Example rounds out the GAG, GSL, and HS microarray binding profiles for AO4B08 scFv (SEQ ID NO: 21), 1HS scFv (SEQ ID NO: 22), and 3HS scFv (SEQ ID NO: 23). Antibody ICLX-1 scFv was previously tested on the glycosphingolipid (GSL) microarray and it failed to exhibit any binding (results not shown).

### Method

The glycosaminoglycan (GAG) and heparan sulfate (HS) microarrays were performed as described in Examples 2-6. The following table summarizes the reagents used.

| | | | Other |
|---|---|---|---|
| Reagent | Vendor | Catalog # | information |
| AO4B08 scFv | Sino Biological | — | 0.58 mg/mL |
| 3HS scFv | Sino Biological | — | 0.57 mg/mL |
| His Tag Antibody, | Sino Biological | 105327-MM02T | — |
| Mouse mAb | | | |
| Goat Anti-Mouse | ThermoFisher | A21436 | — |
| lgG (H + L) AF555 | | | |

To determine whether AO4B08 scFv and 3HS scFv antibodies interact with GAG and HS glycans, the antibody samples were first analyzed using the GAG and HS Glycan microarrays. The microarrays were pretreated with Glycan Array Blocking Buffer (GABB) at room temperature for one hour. After blocking, a pre-complexed mixture of AO4B08 scFv, anti-his tag mAb, and anti-mouse IgG (H+L) AF555 was added to the microarray and incubated at room temperature for 1 hour. The precomplexed mixture initially contained AO4B08 scFv or 3HS scFv at 100 μg/mL concentration, anti-his tag mAb at 1:250 dilution, and anti-mouse IgG (H+L) AF555 at 4 μg/mL concentration. This mixture was then diluted by a factor of two, eleven times. After incubation at room temperature for 1 hour, the microarray was washed and scanned

The following schematic summarizes the steps performed for the GAG microarray:

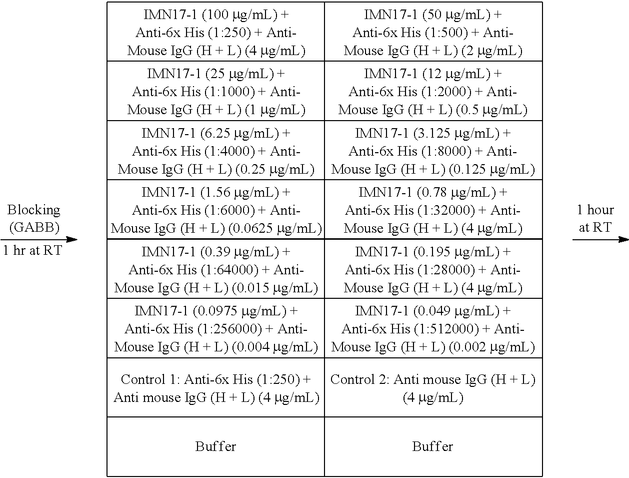

Blocking Buffer: GABB (Glycan Array Blocking Buffer)
Assay buffer GAAB (Glycan Array Assay Buffer)
Working volume: 100 μL/subarray
GAG Array: 16 subarrays, Lot #04132301
   The following schematic summarizes the steps performed for the HS microarray:

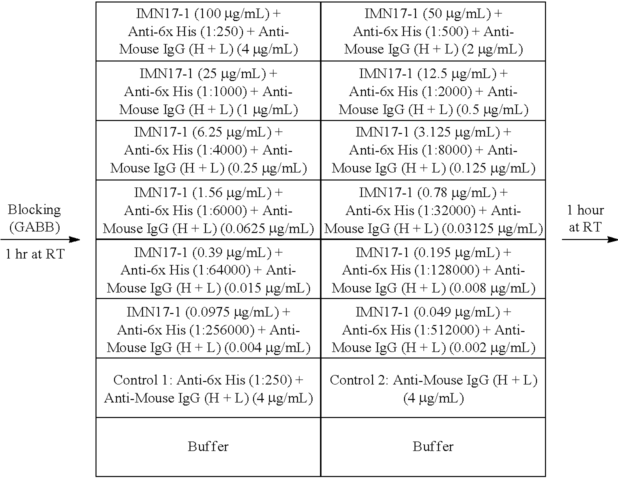

Blocking Buffer: GABB (Glycan Array Blocking Buffer)
Assay buffer: GAAB (Glycan Array Assay Buffer)
Working volume: 100 μL/subarray
HS Array: 16 subarrays, Lot #12022201

After the assay, the microarrays were scanned at 532 nm using high laser intensity (1 PMT). Subsequently, the microarrays were analyzed using Mapix (Innopsys, Carbonne, France) microarray analysis software. As expected, the marker (streptavidin Cy3 and Cy5) and PC3 (mouse IgG,) showed signals.

A similar approach was taken for the glycosphingolipid (GSL) glycan microarrays. The following table summarizes the reagents used.

| | | | Other |
|---|---|---|---|
| Reagent | Vendor | Catalog # | information |
| AO4B08 scFv | Sino Biological | — | 0.58 mg/mL |
| 3HS scFv | Sino Biological | — | 0.57 mg/mL |
| 1HS scFv | Sino Biological | — | 1.72 mg/mL |
| His Tag Antibody, | Sino Biological | 105327-MM02T | — |
| Mouse mAb | | | |
| Goat Anti-Mouse | ThermoFisher | A21436 | — |
| lgG (H + L) AF555 | | | |

The following schematic summarizes the steps performed for the GSL microarray:

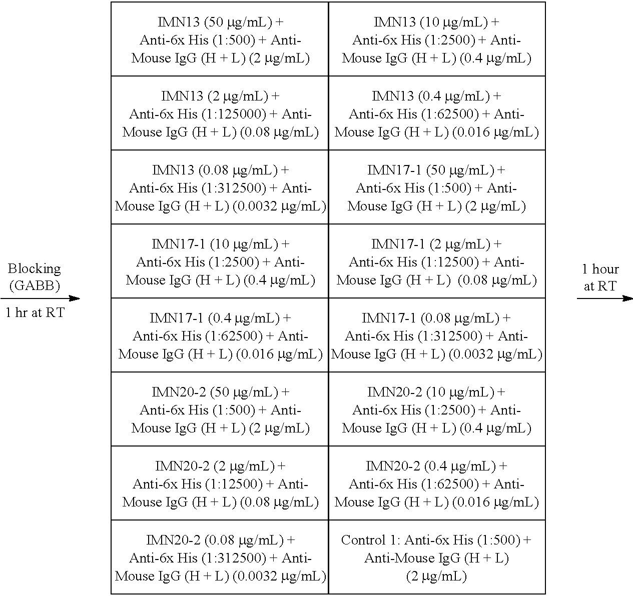

Blocking Buffer: GABB (Glycan Array Blocking Buffer)
Assay buffer GAAB (Glycan Array Assay Buffer)
Working volume: 100 μL/subarray
GSL Array: 16 subarrays, Lot #02062201

### Results

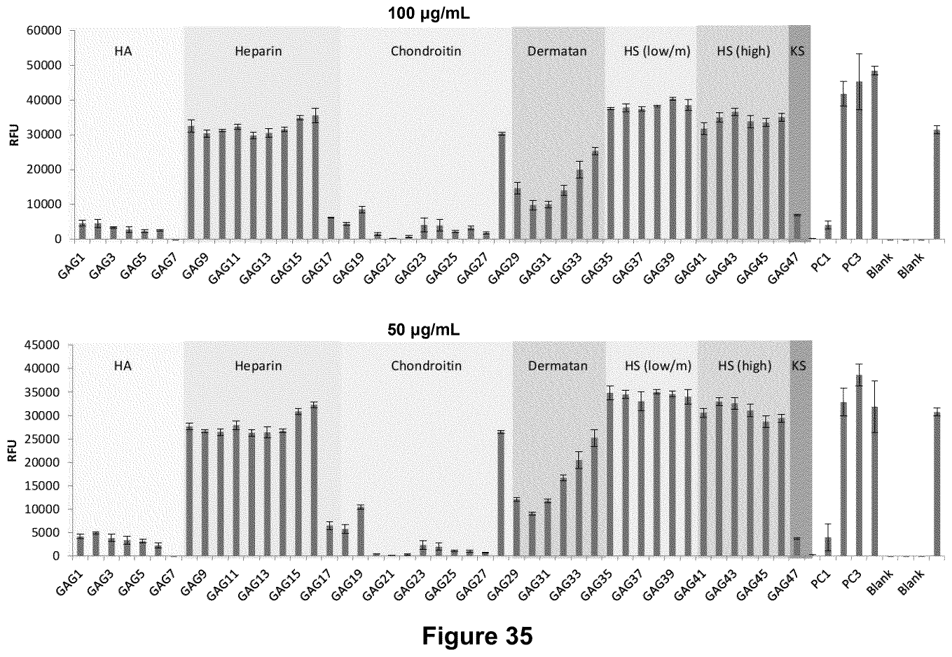

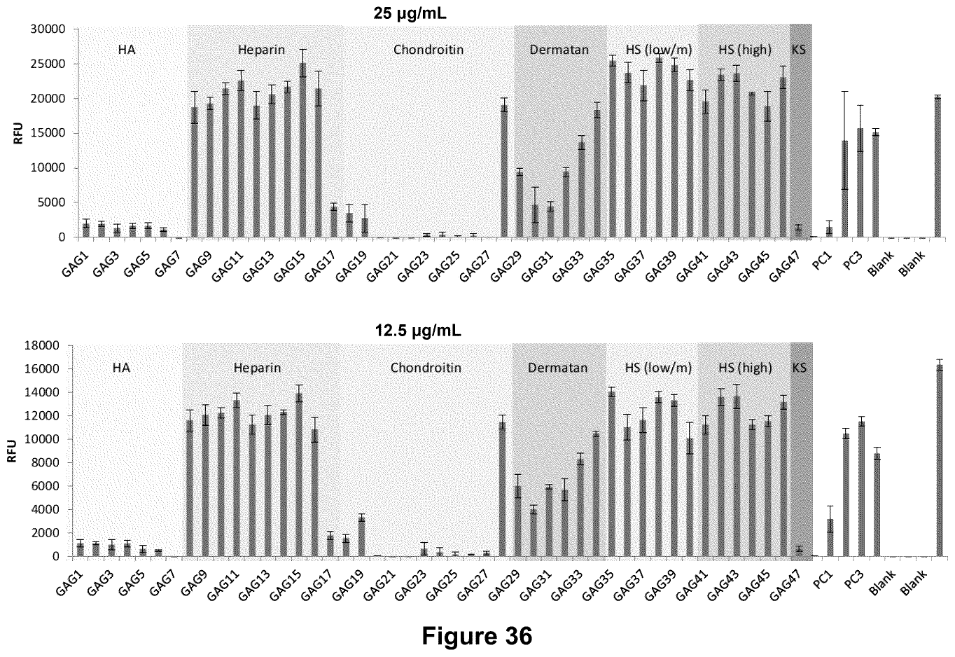

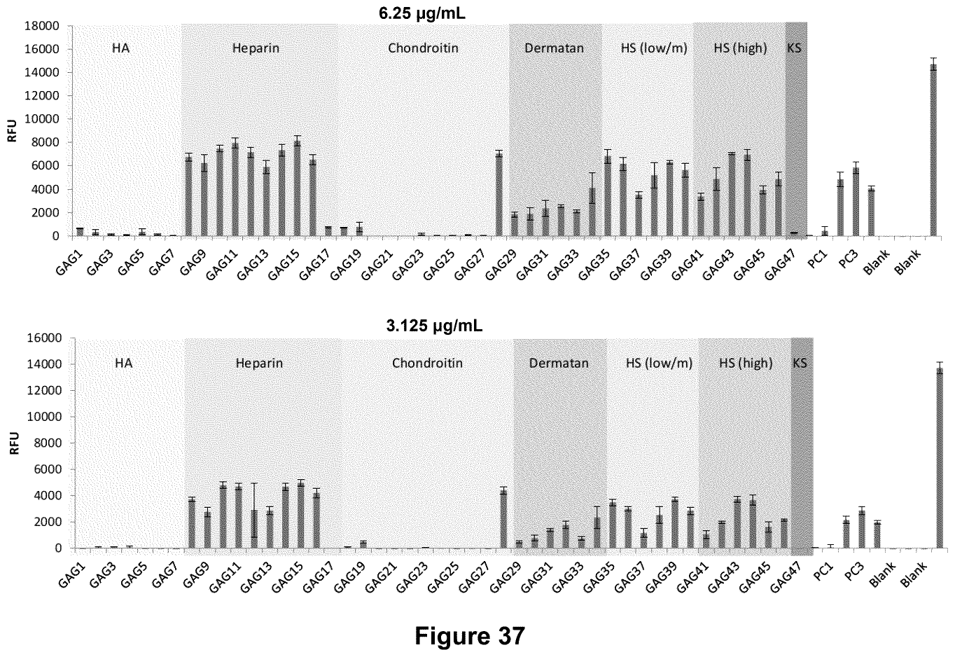

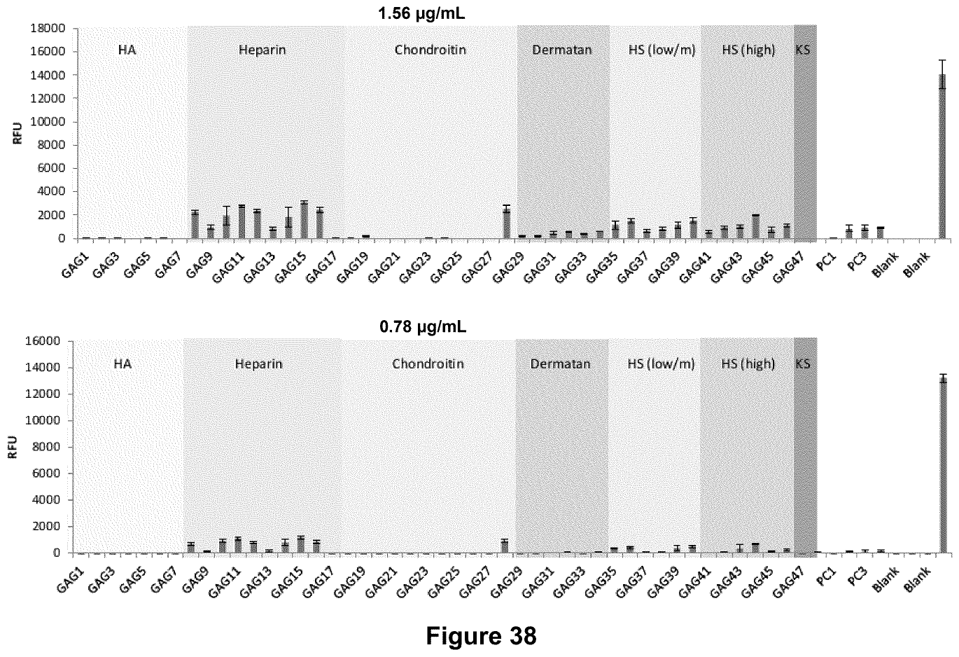

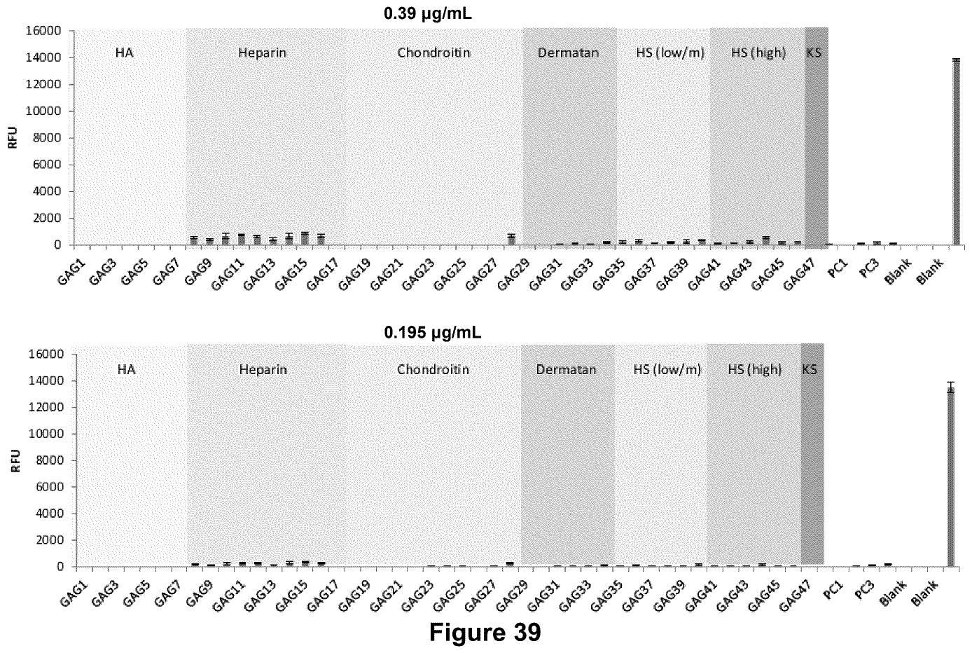

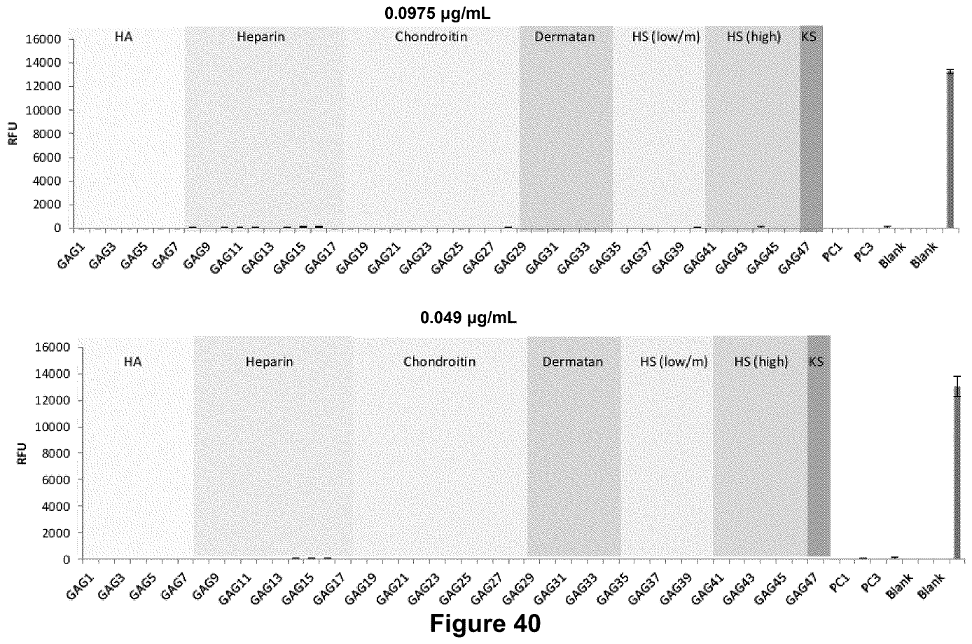

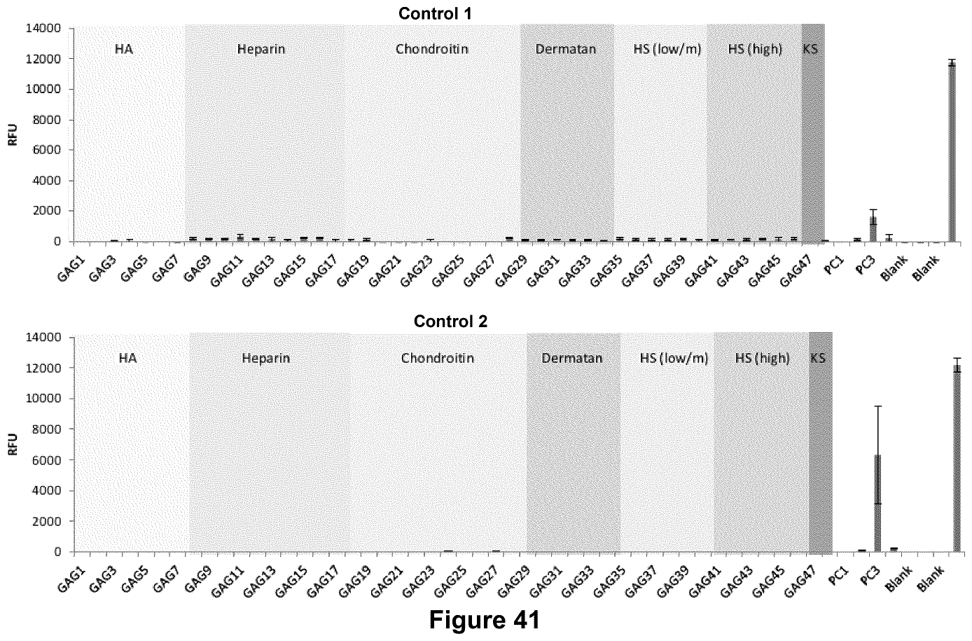

FIGS. 35-40 show that AO4B08 scFv binds to all classes of GAG's printed on the GAG microarray, including some weak binding to hyaluronic acid. Binding signal is strongest for heparin and heparan sulfate molecules. FIG. 41 shows that there was some weak, non-specific binding by the Anti-6×His tag Ab, and anti-mouse IgG (H+L) AF555 Ab interacted with PC3 (mouse IgG).

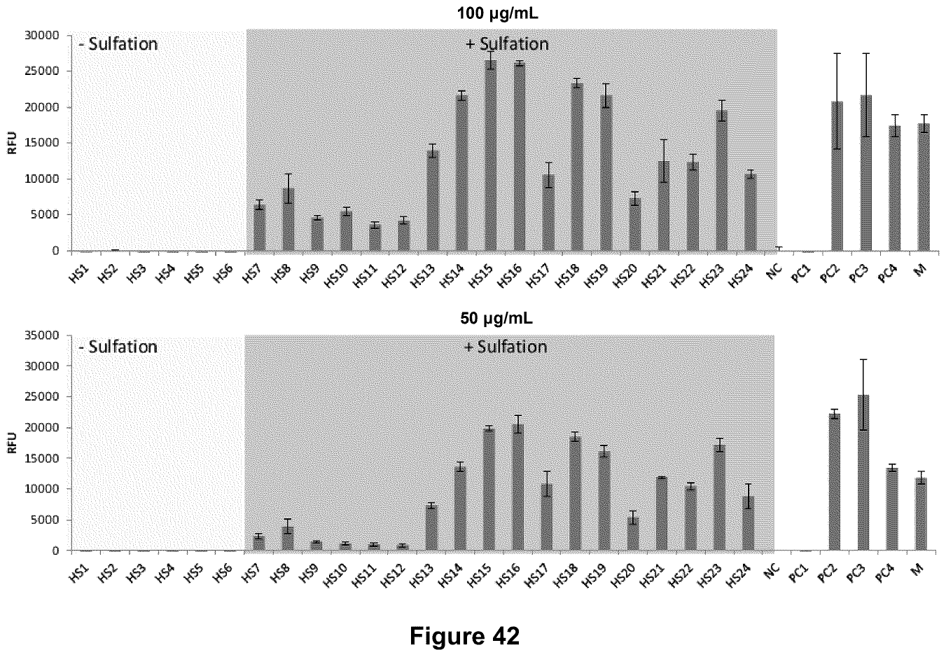

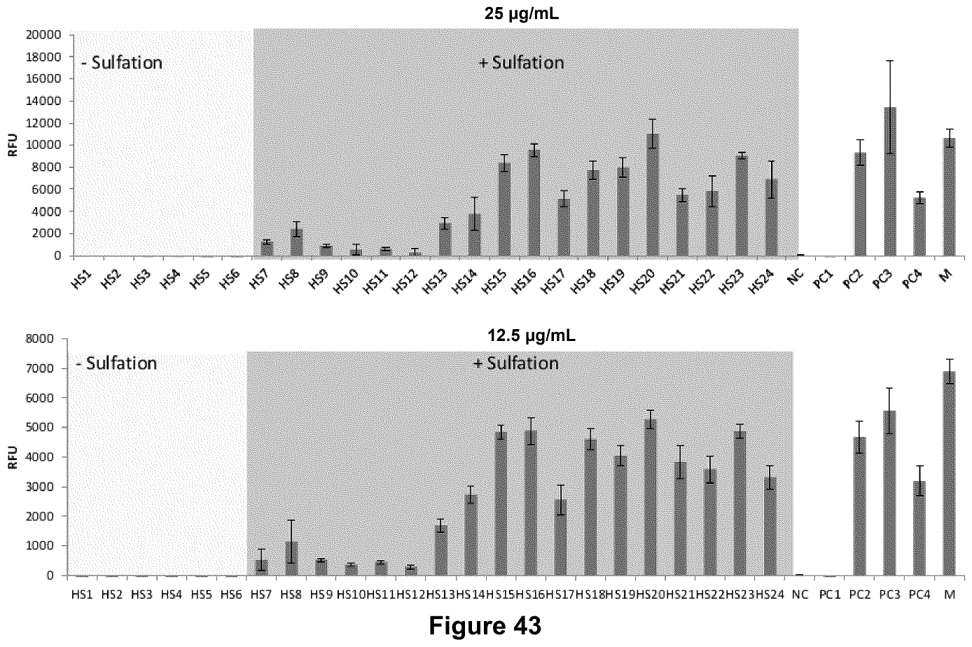

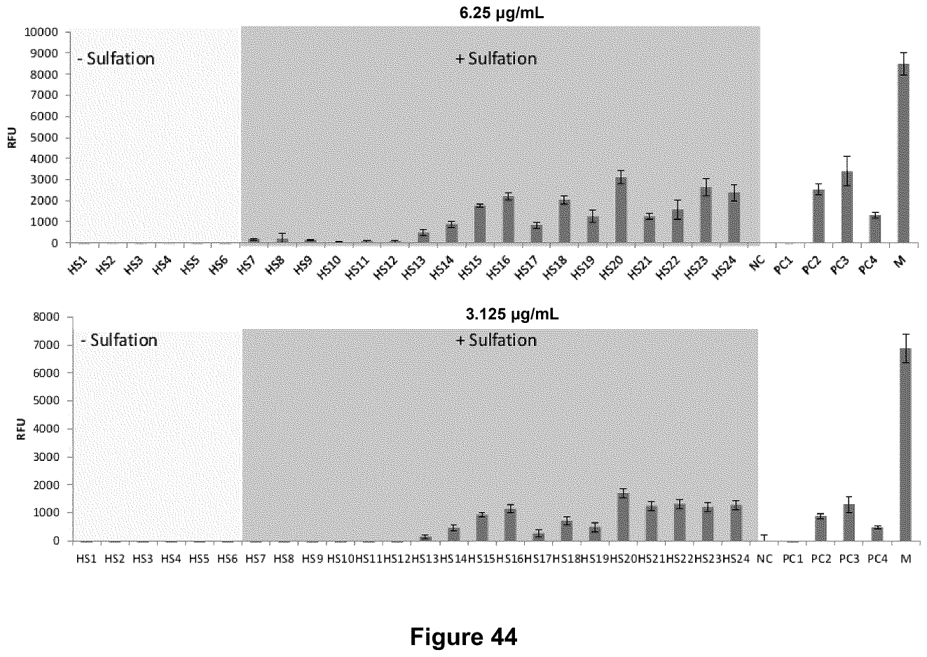

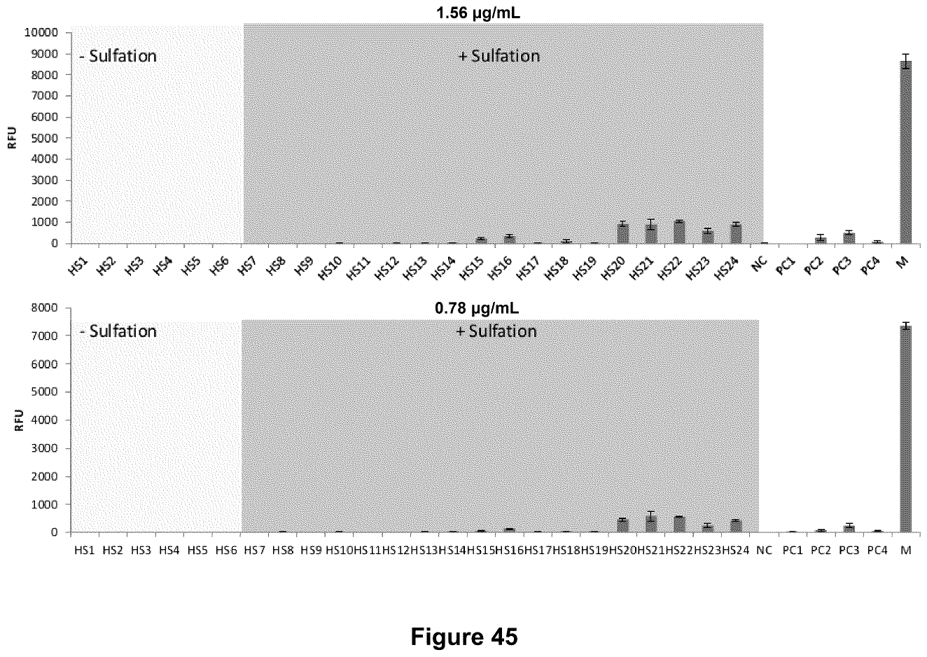

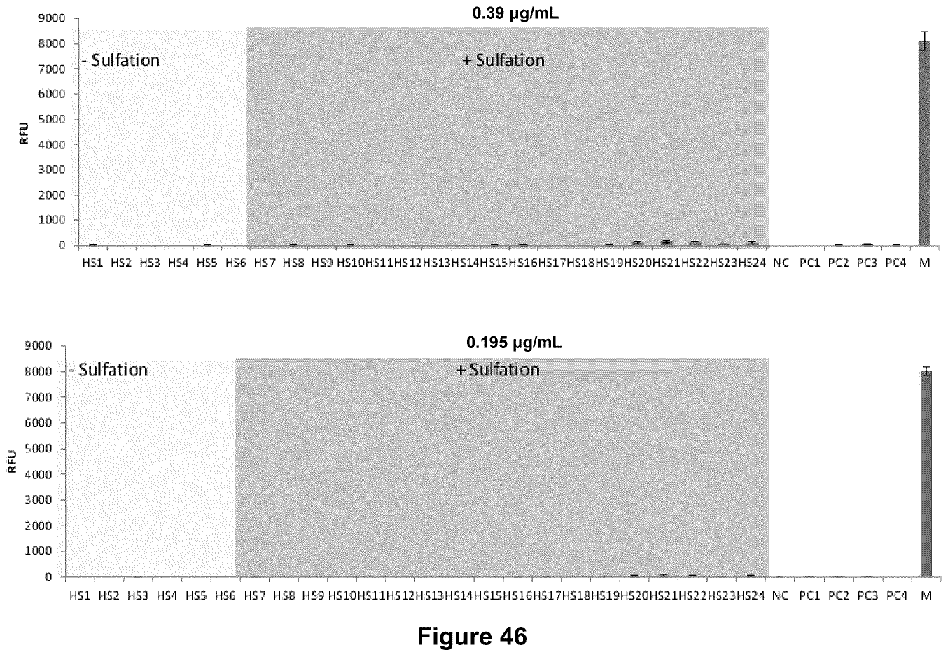

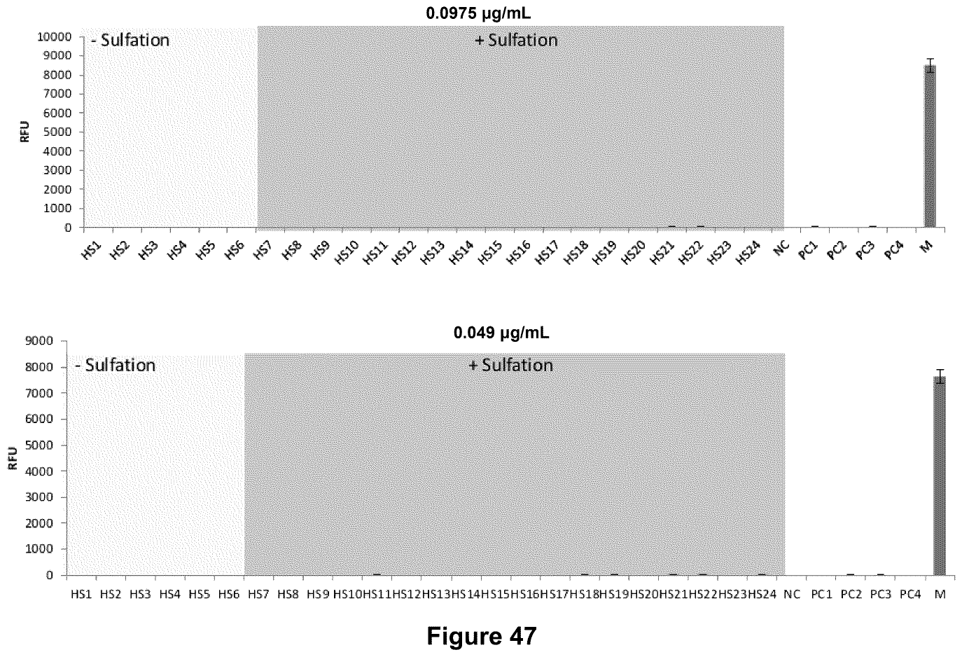

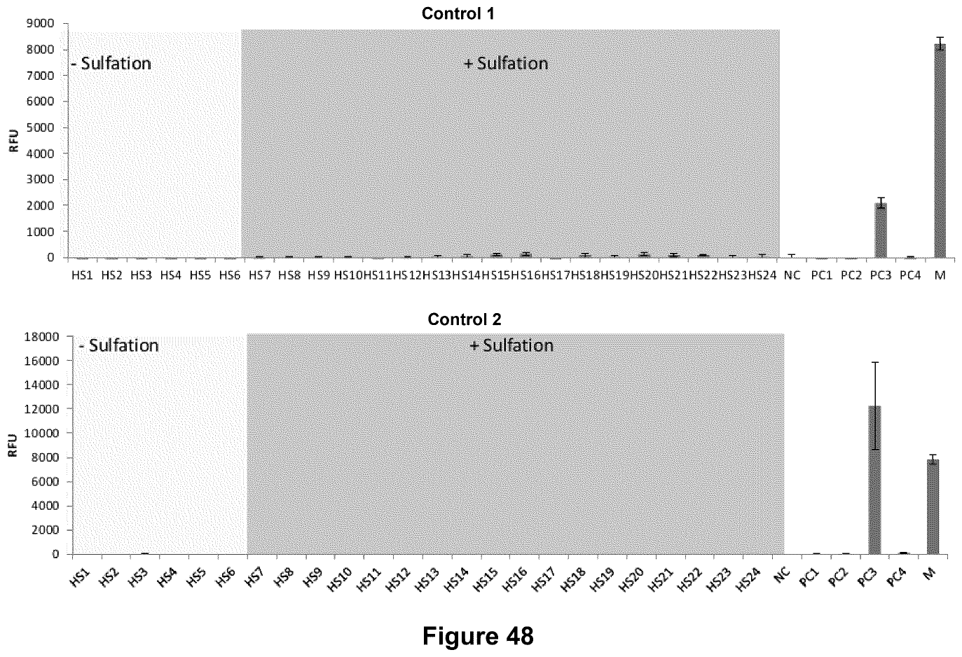

FIGS. 42-47 shows that AO4B08 scFv binds to all sulfated glycan structures on the HS microarray. Binding signal increases as the length of the HS chain increases as well as when the degree of sulfation per disaccharide increases. FIG. 48 shows some weak, non-specific binding by the Anti-6×His tag Ab, and anti-mouse IgG (H+L) AF555 Ab interacted with PC3 (mouse IgG).

These results indicate that the phage display generated antibody, AO4B08 scFv, binds to a variety of glycosaminoglycans (GAGs) and heparan sulfate (HS) structures with specific binding signals observed to a variety of sulfated glycan structures across the GAG and HS microarrays.

On the GAG microarray, AO4B08 scFv bound to every class of GAG molecules printed on the microarray. Binding signal was strongest for the heparin and heparan sulfate classes of GAGs. Binding signal strength did not vary highly as chain length and degree of sulfation changed. Interestingly, AO4B08 scFv also showed a weak interaction with hyaluronic acid (HA) at high concentrations on the microarray. This is the first tested antibody showing interaction with a non-sulfated glycan structure. Interestingly, binding signal by AO4B08 scFv to HA molecules decreased as the molecule chain length increases and was fully diminished when the chain length was very large (ex: GAG7).

As predicted, AO4B08 scFv's binding profile on the HS microarray was similar to other tested antibodies as AO4B08 scFv bound to all of the sulfated glycans on the microarray and did not bind to the non-sulfated glycans. In general, binding signal increased as the chain length and degree of sulfation per disaccharide increased on the heparan sulfate glycans. Compared to the other tested antibodies, binding to GAGs and HS glycans has a higher point of saturation.

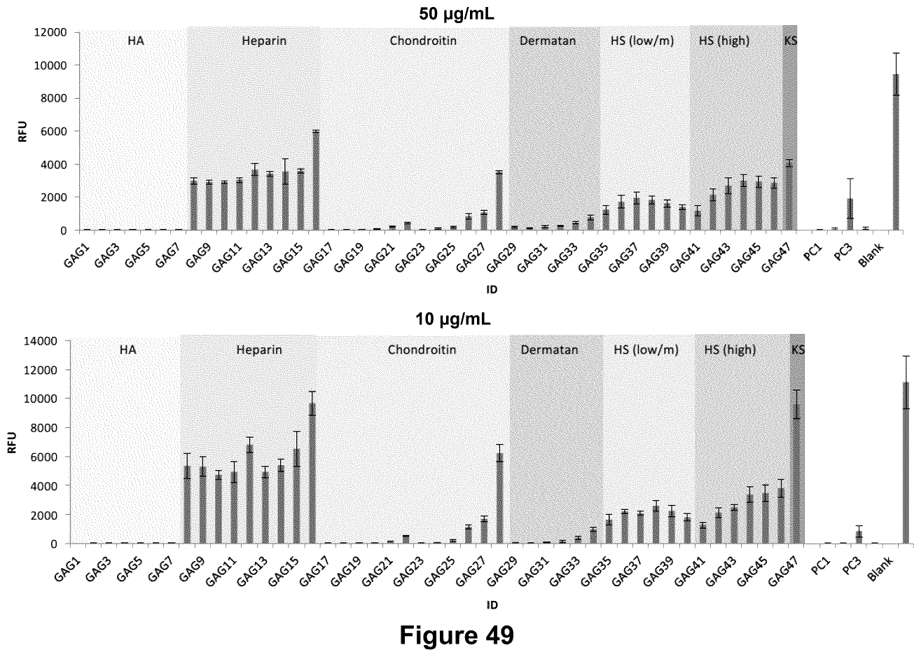

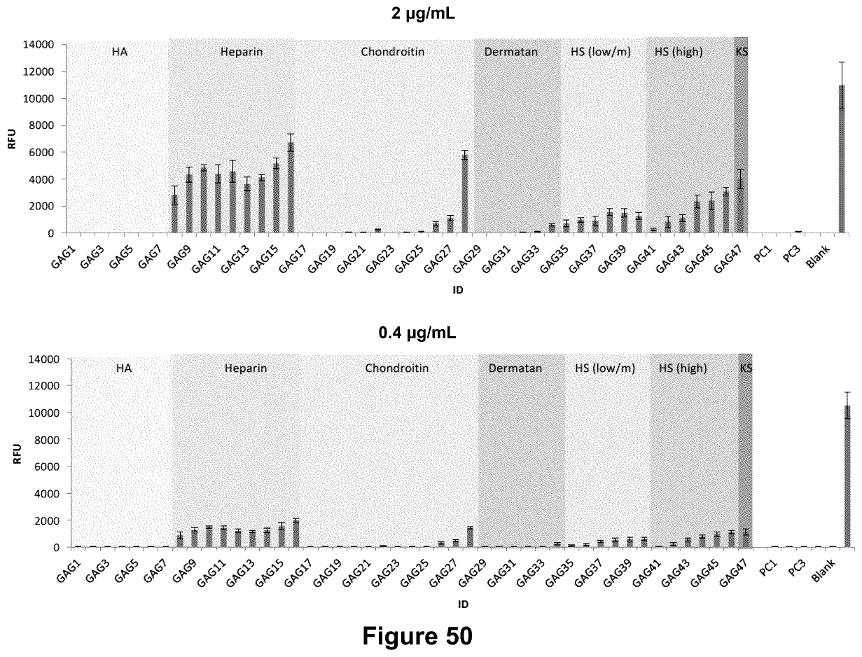

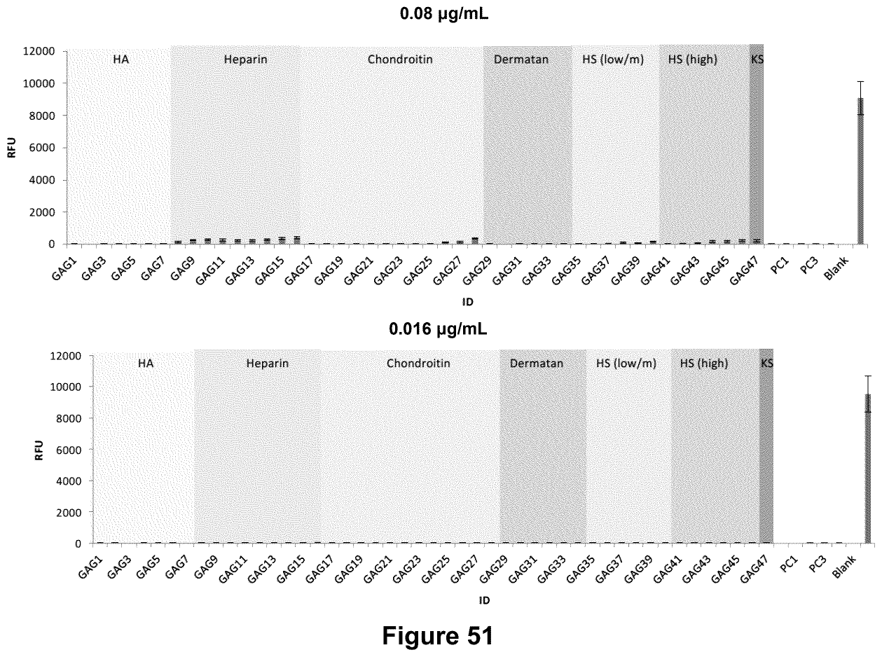

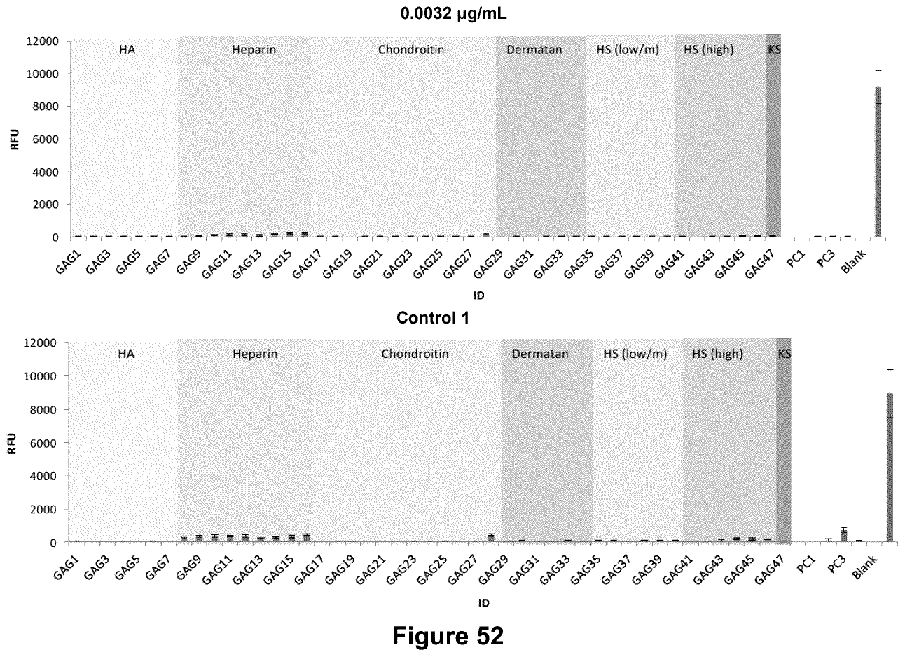

FIGS. 49-52 show the GAG binding profile of 3HS scFv at concentration ranging from 50 μg/mL to 0.0032 μg/mL. The results show that 3HS scFv binds mainly to heparin and heparan sulfate. 3HS scFv also binds to long chain chondroitin sulfate, keratan sulfate, and weakly binds to dermatan sulfate. There is a positive trend in binding signal to molecules of greater chain length and sulfation. The control performed as expected (FIG. 52). There was a small interaction between the Anti-6×His antibody and some of the GAGs on the microarray. Anti-mouse IgG (H+L) interacted with PC3 (mouse IgG).

As expected, none of the tested antibodies showed any binding on the GSL microarray (data not shown). As described above, Fernández-Marrero et al, (2011), offers only a single example of an anti-GAG antibody (i.e., chP3R99) that is cytotoxic to cancer cells in vitro. However, it is the opinion of Fernández-Marrero et al, (2011), that said cytotoxicity is due to binding of chP3R99 to NeuGcGM3 and not to any GAG. The present disclosure shows that the 3 polyspecific anti-GAG antibodies, AO4B08 scFv, 1HS scFv, and 3HS scFv bind only to GAGs and not to NeuGcGM3 or any other GSL.

### Example 12: Cell Death Analysis of RPMI 8226 Cells Treated with AO4B08 scFV, 1HS scFv, and 3HS scFv

The effect of antibodies AO4B08 scFV (SEQ ID NO:21), 1HS scFv (SEQ ID NO: 22), and 3HS scFv (SEQ ID NO: 23) on RPMI 8226 viability was definitively assessed using of flow cytometry and PI-stained cells. The method used is as described in Example 9. Additional information regarding the effect of these 3 antibodies on apoptosis was determined using an Annexin/7-ADD apoptosis kit.

### Method

The effect AO4B08 scFv, 1HS scFv, and 3HS scFv treatment was evaluated based on co-incubation of RPMI 8226 cells with five different concentrations of the three different antibodies followed by cell death assay by flow cytometry.

Culturing conditions: 10,000 cells in 100 μL 10% FBS/RPMI 1640 media, cultured at 37° C., 5% CO₂ for 4 days.

Annexin/7-AAD Apoptosis kit (Sigma, cat #CBA059) was used for flow cytometry analysis to define dead, apoptotic and live cells (FIG. 55). Briefly, the following steps were performed:
Spin down the plate at 500 g for 5 minutes.
Wash cells twice with 200 μL cold DPBS.
Resuspend into 50 μL of Annexin V Binding Buffer containing 5 μL/per sample of APC Annexin V. Mix well.
Add 50 μL of Annexin V Binding Buffer containing 5 μL/per sample of 7-AAD. Mix well.
Incubate for 15 min at room temperature in the dark.
Add 400 μL of Annexin V Binding Buffer to each well by transferring samples into 96 deep well plate.
Analyze by flow cytometry with proper machine settings.

### Results

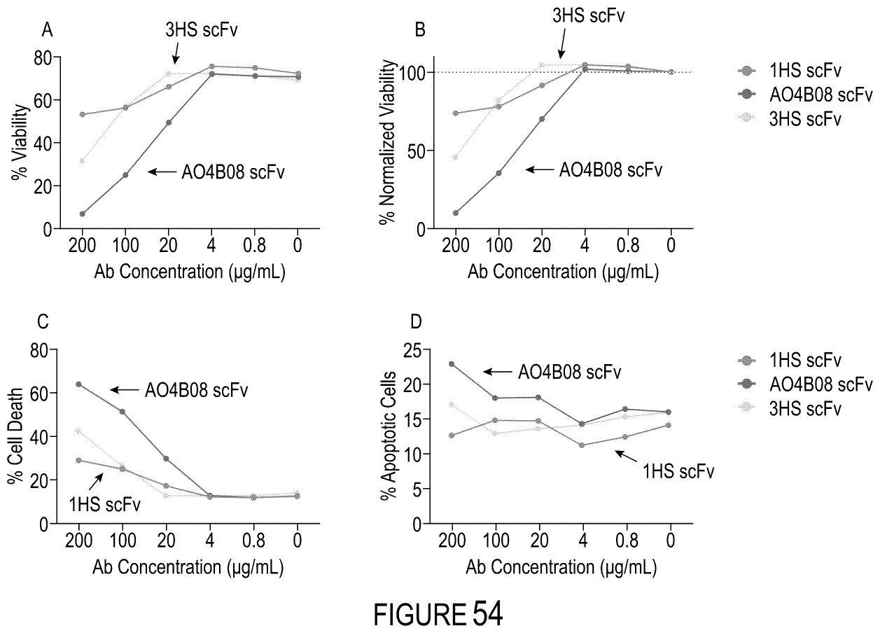

As shown in FIGS. 54A and 54B, the viability reduction was greatest with AO4B08 scFv, decreasing to only 7.01% at a concentration of 200 μg/mL. The reduction in cell viability was non-significant s 20 μg/mL for 3HS scFv and 5 4 μg/mL for 1HS scFv and AO4B08 scFv.

The percentage of cell death increased in correlation with scFv concentrations, with the greatest effect observed again with AO4B08 scFv (63.6% at 200 μg/mL). The cytotoxic effect of the scFvs became insignificant s 20 μg/mL for 3HS scFv and 5 4 μg/mL for 1 HS scFv and AO4B08 scFv. The difference between apoptotic cells across different concentrations were non-significant.

All three antibodies showed some degree of direct cytotoxicity towards RPMI 8226 cells in positive correlation with increasing the antibody concentration. AO4B08 scFv showed the greatest cytotoxic effect, reaching 7.01% viability and 63.6% cell death at the highest concentration of 200 μg/mL. Cytotoxicity was not observed 5 4 μg/mL for both AO4B08 scFv and 1HS scFv whereas cytotoxicity was not observed ≤20 μg/mL for 3HS scFv. With respect to the negative control, untreated cells showed ~70% viability, ~16% apoptosis and ~13% cell death after the 4-day culture.

The results of this experiment should be viewed in the context of Examples 9 and 10, as described above. Collectively, all 3 Examples offer strong support for a generalized, in vitro use of polyspecific anti-GAG antibodies to kill cancer cells. Unlike antibody chP3R99, which binds to both NeuGcGM3 and multiple classes of GAG and which is cytotoxic for the NeuGcGM3-positive murine leukemia line L1210 via its anti-NeuGcGM3 binding properties, none of the 4 antibodies described in Examples 9, 10, 11, and 12 bind to NeuGcGM3.

Thus, polyspecific anti-GAG antibodies, in a general sense, are good candidates for the direct killing of cancer cells in vitro. And previous results with the polyspecific anti-GAG antibody, NovoMAb-G2 scFv, (which wasn't understood to be a polyspecific anti-GAG antibody at the time) suggest that polyspecific anti-GAG antibodies, in a general sense, may also be good candidates for in vivo anti-cancer applications. This latter assumption is based on the observation that NovoMAb-G2 scFv was able to image tumors in vivo in pre-clinical models and in humans, and also was also able to inhibit tumor growth and metastases in vivo in preclinical models as well as induce a partial response in 4 of 12 patients with advanced metastatic disease in a Phase I/II study.

### Ex Vivo Applications

Although the present disclosure does not add further to our understanding of polyspecific anti-GAG antibodies as in vivo anti-cancer agents, it's now clear that specific groups of cancer patients could potentially benefit from polyspecific anti-GAG antibodies applied ex vivo, in the context of leukapheresis and hematopoietic stem cell transplantation.

Hematopoietic stem cell (HSC) transplantation, specifically autologous stem cell transplantation (ASCT), remains a cornerstone in the treatment of multiple myeloma (MM), despite the advent of newer pharmacological agents. The current status of ASCT in MM involves several key aspects, including its efficacy, advancements in treatment protocols, and ongoing research into improving outcomes.

ASCT is still considered the standard of care for eligible newly diagnosed patients with MM. It has been shown to improve progression-free survival (PFS) and, in some cases, overall survival (OS) when used in conjunction with high-dose chemotherapy (Devarakonda et al, 2021; Al Hamed et al., 2019). The procedure involves collecting the patient's own stem cells, administering high-dose chemotherapy to eradicate cancerous cells, and then reinfusing the stem cells to restore bone marrow function (Devarakonda et al, 2021; Al Hamed et al., 2019; https://bethematch.org/patients-and-families/about-transplant/blood-cancers-and-diseases-treated-by-transplant/multiple-myeloma/).

Recent advancements in the field of MM treatment have focused on integrating ASCT with novel therapeutic agents to enhance outcomes. The use of induction therapy with newer agents like proteasome inhibitors (e.g., bortezomib), immunomodulatory drugs, and monoclonal antibodies before ASCT has led to deeper remissions and potentially longer survival times (Devarakonda et al, 2021; Al Hamed et al., 2019). Maintenance therapy following ASCT, particularly with drugs like lenalidomide, has become a common practice to prolong remission (Devarakonda et al, 2021; Al Hamed et al., 2019).

Ongoing research is exploring various strategies to optimize the timing and efficacy of ASCT. This includes studies on the use of tandem ASCT (two consecutive transplants) for high-risk patients and the potential benefits of incorporating newer agents like daratumumab into the treatment regimen before and after transplantation (Devarakonda et al, 2021; Al Hamed et al., 2019). Additionally, there is significant interest in understanding the role of minimal residual disease (MRD) status in guiding therapy post-ASCT (Tilmont et al., 2023).

Despite its benefits, ASCT is associated with significant challenges, including the risk of relapse and the physical toll of high-dose chemotherapy. The procedure is generally recommended for younger and fit patients due to its intensive nature. Moreover, the emergence of highly effective non-transplant therapies has led some experts to reconsider the timing of ASCT, with some suggesting it may be delayed until first relapse in certain cases (Al Hamed et al., 2019).

In summary, ASCT remains a vital component of MM treatment, particularly for newly diagnosed patients. Its integration with novel therapeutic agents and ongoing research into optimizing its use continue to improve outcomes for patients with MM. However, the treatment landscape is dynamic, and continuous evaluation of ASCT's role in the era of new therapies is essential (Devarakonda et al, 2021; Al Hamed et al., 2019; https://bethematch.org/patients-and-families/about-transplant/blood-cancers-and-diseases-treated-by-transplant/multiple-myeloma/).

The current status of purging in autologous bone marrow transplantation (ABMT) for multiple myeloma (MM) is characterized by ongoing debate and research into its efficacy and impact on patient outcomes. Purging is the process of removing residual myeloma cells from the autograft to reduce the risk of relapse post-transplantation. Despite its theoretical benefits, the practice of purging has not been universally adopted due to mixed evidence regarding its effectiveness.

The concept of purging is based on the observation that relapse in MM patients post-ABMT may be due to the reinfusion of residual tumor cells within the graft. However, the actual impact of purging on relapse rates and disease-free survival is still under investigation.

Some studies have shown that lower levels of graft contamination with myeloma cells are associated with better outcomes, suggesting that even low levels of contamination can influence the course of the disease (Bartee et al., 2012; Lee et al., 2019).

Conversely, other studies and editorials have pointed out that there is limited data supporting the notion that purging autografts has a favorable effect on relapses or disease-free survival (Bensinger, 1998; Benowitz, 2000). Certain purging techniques may also have adverse effects, such as delayed hematopoietic or T cell reconstitution.

Various methods of ex vivo purging have been described, including CD34+ cell selection, chemotherapeutic agents, viral-based methods, and monoclonal antibodies (Lee et al., 2019; Hong Yang et al., 2011; Bartee et al., 2012; Anderson et al., 1991).

A study evaluated the efficacy of purging myeloma cells using bortezomib or lenalidomide, showing that these agents could effectively reduce myeloma cell contamination (Bartee et al., 2012; Lee et al., 2019).

Another approach involved the use of an oncolytic poxvirus called myxoma virus (MYXV) for selective elimination of myeloma cells from autografts, which showed promise in ex vivo treatment (Lee et al., 2019). Despite these developments, no definitive purging method has been generally adopted in clinical settings (Bartee et al., 2012).

The value of purging remains controversial among bone marrow transplant specialists, with some arguing that no definitive evidence shows that purging improves patient survival (Benowitz, 2000). Technical challenges and a lack of clinical trial data showing a survival benefit of purging versus non-purging have contributed to the ongoing debate (Benowitz, 2000). The effectiveness of purging may also depend on the efficacy of the chemotherapy regimen used to treat the patient.

In summary, while purging is an appealing concept aimed at reducing relapse rates by eliminating residual myeloma cells from autografts, its adoption in clinical practice has been limited by the lack of conclusive evidence demonstrating a clear benefit in terms of survival or disease-free survival. Ongoing research and well-designed clinical trials are needed to clarify the role of purging in ABMT for MM and to establish whether it should be a standard component of the transplantation process.

The present disclosure proposes a new tool for purging an autologous stem cell transplantation (ASCT) or an autologous bone marrow transplantation (ABMT) of multiple myeloma (MM) or neuroblastoma cells, as supported by the examples described herein. In addition, patients with Hodgkin lymphoma could also benefit from ex vivo purging as the heparan sulfate proteoglycan (HSPG), syndecan-1 (SDC1), is upregulated in classical Hodgkin lymphoma (cHL), and SDC4 is transcriptionally overexpressed in many Hodgkin lymphoma cell lines (e.g., KM-H2 and L-540) (Gharbaran, 2016). As shown in the present disclosure, polyspecific anti-GAG antibodies, such as ICLX-1 scFv, are highly effective at in vitro killing of RPMI 8226 multiple myeloma cells, which characteristically overexpress syndecan-1.

While the invention has been described in connection with specific embodiments thereof, it will be understood that the scope of the claims should not be limited by the preferred embodiments set forth in the examples but should be given the broadest interpretation consistent with the description as a whole.

### REFERENCES

Al Hamed, R., Bazarbachi, A. H., Malard, F. et al. Current status of autologous stem cell transplantation for multiple myeloma. Blood Cancer J. 9, 44 (2019).
Al Mughram M H, Catalano C, Herrington N B, Safo M K, and Kellogg G E. 3D interaction homology: The hydrophobic residues alanine, isoleucine, leucine, proline and valine play different structural roles in soluble and membranes proteins. Front Mol Biosci 10:1116868, 2023.
Alfonso M, Díaz A, Hernández A M, Pérez A, Rodrigez E, Bitton R, Pérez R, and Vázquez A M. An anti-idiotype vaccine elicits a specific response to N-glycolyl sialic acid residues of glycoconjugates in melanoma patients. J Immunol 168:2523-2529, 2002.
Alfonso S, Díaz R M, de la Torre A, Santiesteban E, Aguirre F, Pérez K, Rodriguez J L, Barroso M C, Hernández A M, Toledo D, Gabri M R, Alonso D F, Viada C, Gómez R E, Pestana E, Suarez E, Vázquez A M, Pérez R, and Macías A. 1E10 anti-idiotype vaccine in non-small cell lung cancer: experience in stage IIIb/IV patients. Cancer Biol Therapy 6(12):1847-52, 2007.
Altinbas M, Coskun H S, Er O, Ozkan M, Eser B, Unal A, Cetin M, and Soyuer S. A randomized clinical trial of combination chemotherapy with and without low-molecular-weight heparin in small cell lung cancer. J Thromb Haemost 2:1266-1271, 2004.
Altinbas M, Dikilitas M, Ozkan M, Dogu G G, Er O, and Coskun H S. The effect of small-molecular-weight heparin added to chemotherapy on survival in small-cell lung cancer—A retrospective analysis. Indian J Cancer 51(3):324-329, 2014.
Anderson K C, Barut B A, Ritz J, Freedman A S, Takvorian T, Rabinowe S N, Soiffer R, Heflin L, Coral F, Dear K; Monoclonal antibody-purged autologous bone marrow transplantation therapy for multiple myeloma. Blood 1991; 77 (4): 712-720.
Atallah J, Khachfe H H, Berro J, and Assi H. The use of heparin and heparin-like molecules in cancer treatment: a review. Cancer Treatment and Res Comm 24:100192, 2020.
Ay C, Kamphuisen P W, and Agnelli G. Antithrombotic therapy for prophylaxis and treatment of venous thromboembolism in patients with cancer: review of the literature on current practice and emerging options. ESMO Open 2(2):e000188, 2017.
Bartee E, Chan W M, Moreb J S, Cogle C R, McFadden G. Selective purging of human multiple myeloma cells from autologous stem cell transplantation grafts using oncolytic myxoma virus. Biol Blood Marrow Transplant. 2012 October; 18(10):1540-51.
Benowitz S, Bone Marrow Experts Are Still Debating the Value of Purging, JNCI: Journal of the National Cancer Institute, Volume 92, Issue 3, 2 Feb. 2000, Pages 190a-192.
Bensinger, W. Should we purge? Bone Marrow Transplant 21, 113-115 (1998).
Borsig L. Heparin as an inhibitor of cancer progression. Published in Progress in Molecular Biology and Translational Science, pp 335-349, 2010.
Brito V, Mellal K, Zoccal K F, Soto Y, Menard L, Sarduy R, Faccioli L H, Ong H, Vázquez A M, and Marleau S. Atheroregressive potential of the treatment with a chimeric monoclonal antibody against sulfated glycosaminoglycans on pre-existing lesions in apolipoprotein E-deficient mice. Front Pharmacol 8:782, 2017.
Cardin A D, and Weintraub H J. Molecular modeling of protein-glycosaminoglycan interactions. Arteriosclerosis 9(1):21-32, 1989.
Chen C, Zhao S, Karnad A, and Freeman J W. The biology and role of CD44 in cancer progression: therapeutic implications. J Hematol Oncol 11:64-87, 2018.
Chen J S, Lan K, and Hung M C. Strategies to target HER-2/Neu overexpression for cancer therapy. Drug Resist. Updates 6:129-36, 2003.
Christianson H C, van Kuppevelt T H, and Belting M. ScFv anti-heparan sulfate antibodies unexpectedly activate endothelial and cancer cells through p38 MAPK: implications for antibody-based targeting of heparan sulfate proteoglycans in cancer. PLOS ONE 7(11):e49092, 2012.
Dan M. “Human anti-glioma monoclonal antibodies from patients with neurological tumors”. Doctor of Philosophy (Division of Experimental Medicine) dissertation, McGill University, 1989.
Dan M, Schlachta C M, Guy J, McKenzie R G, Dorscheid D R, Sandor V A, Villemure J-G, and Price G B. Human antiglioma monoclonal antibodies from patients with astrocytic tumors. J Neurosurg 76(4):660-9, 1992.
Delcommenne M and Klingemann H-G. Detection and characterization of syndecan-1-associated heparan sulfate 6-O-sulfated motifs overexpressed in multiple myeloma cells using single chain antibody variable fragments. Human Antibodies 21:29-40, 2012.
Dennissen MABA, Jenniskens G J, Pieffers M, Versteegt EMM, Petitou M, Veerkamp J H, and van Kuppevelt T H. Large, tissue-regulated domain diversity of heparan sulfates demonstrated by phage display antibodies. J Biol Chem 277(13):10982-10986, 2002.
Devarakonda S, Efebera Y, Sharma N. Role of Stem Cell Transplantation in Multiple Myeloma. Cancers (Basel). 2021 Feb. 18; 13(4):863. doi: 10.3390/cancers13040863.
Fannon M, Forsten K E, and Nugent M A. Potentiation and inhibition of bFGF binding by heparin: a model for regulation of cellular response. Biochemistry 39:1434-1445, 2000.
Fernández-Marrero Y, Hernández T, Roque-Navarro L, Talavera A, Moreno E, Griñan T, Vásquez A M, Mateo de Acosta C, Pérez R, and López-Requena A. Switching on cytotoxicity by a single mutation at the heavy chain variable region of an anti-ganglioside antibody. Mol Immunol 48:1059-1067, 2011.
Franchini M, and Mannucci P M. Low-molecular-weight heparins and cancer: focus on antitumoral effect. Ann Med 47:116-121, 2015.
Gandhi N S and Mancera R L. The structure of glycosaminoglycans and their interactions with proteins. Chem Biol Drug Des 72:455-482, 2008.
Gharbaran, R. Insights into the molecular roles of heparan sulfate proteoglycans (HSPGs-syndecans) in autocrine and paracrine growth factor signaling in the pathogenesis of Hodgkin's lymphoma. Tumor Biol. 37, 11573-11588 (2016).
György B, Tóthfalisi L, Nagy G, Pásztói M, Géher P, Lörinc Z, Polgár A, Rojkovich B, Ujfalussy I, Poór G, Pócza P, Wiener Z, Misják P, Koncz A, Falus A, and Buzás E. Natural autoantibodies reactive with glycosaminoglycans in rheumatoid arthritis. Arthritis Res Therapy 10:R110, 2008.
Hao G, Xu Z P, and Li L. Manipulating extracellular tumour pH: an effective target for cancer therapy. RSC Adv 8:22182, 2018.
Hong Yang, Simon N. Robinson, Yago Nieto, Richard J. Jones, Christopher D. Gocke, Junjun Lu, Sergio A. Giralt, Roy B. Jones, William K. Decker, Dongxia Xing, David Steiner, Richard E. Champlin, John D. McMannis, Jingjing Ng, Michael W. Thomas, Nina Shah, Borje S. Andersson, Simrit Parmar, Elizabeth J. Shpall; Ex Vivo Graft Purging and Expansion of Autologous Blood Progenitor Cell Products from Patients with Multiple Myeloma. Cancer Res 15 Jul. 2011; 71 (14): 5040-5049.
Huhle G, Harenberg J, Malsch R, and Heene D L. Monoclonal antibodies against heparin and heparinoids. Seminars Thrombosis Hemostasis 23(1):17-22, 1997.
Icli F, Akhbulut H, Utkan G, Yalcin B, Dincol D, Isikdogan A, Demirkazik A, Onur H, Cay F, and Buyukcelik A. Low molecular weight heparin (LMWH) increases the efficacy of cisplatinum plus gemcitabine combination in advanced pancreatic cancer. J Surg Oncol 95:507-512, 2007.
Jenniskens G J, Oosterhof A, Brandwijk R, Veerkamp J H, van Kuppevelt T H. Heparan sulfate heterogeneity in skeletal muscle basal lamina: demonstration by phage display-derived antibodies. J Neurosci. 2000 Jun. 1, 20(11):4099-111.
Kabat E. A., Wu T. T., Perry M., Gottesman K. S., and Foeller C. Sequences of Proteins of Immunological Interest, 5th Ed. Public Health Service, National Institutes of Health, Bethesda, M D. (1991).
Katoh H, Komura D, Hiroki K, Suzuki R, Yamamoto A, Kakiuchi M, Sato R, Ushiku T, Yamamoto S, Tatsuno K, Oshima T, Nomura S, Seto Y, Fukayama M, Aburatani H, and Ishikawa S. Immunogenetic profiling for gastric cancers identifies sulfated glycosaminoglycans as major and functional B cell antigens in human malignancies. Cell Reports 20:1073-1087, 2017.
Kerever A, and Arikawa-Hirasawa E. Optimal extracellular matrix niches for neurogenesis: identifying glycosaminoglycan chain composition in the subventricular neurogenic zone. Front Neuroanat 15:764458, 2021.
Klamer Z and Haab B. Combined analysis of multiple glycan-array datasets: new explorations of protein-glycan interactions. Anal Chem 93(31):10925-10933, 2021.
Klaus T, and Deshmukh S. pH-responsive antibodies for therapeutic applications. J Biomed Sci 28:11, 2021.
Kurup S, Wijnhoven T J M, Jenniskens G, Kimata K, Habuchi H, Li J-P, Lindahl U, van Kuppevelt T H, and Spillmann D. Characterization of anti-heparan sulfate phage display antibodies AO4B08 and HS4E4. J Biol Chem 282(29):21032-21042, 2007.
Kyte J and Doolittle R F. A simple method for displaying the hydropathic character of a protein. J Mol Biol 157:105-132, 1982.
Laidman, J. “Setbacks and success”. The Toledo Blade, Feb. 14, 2002.
Lebeau B, Baud M, Masanes M-J, Febvre M, Mokhtari T, and Chouaïd C. Optimization of small-cell lung cancer chemotherapy with heparin: a comprehensive retrospective study of 239 patients treated in a single specialized center. Chemotherapy 57:253-258, 2011.
Lebeau B, Chastang C, Brechot J-M, Capron F, Dautzenberg B, Delaisements C, Mornet M, Brun J, Hurdebourcq J-P, and Lemaire E. Subcutaneous heparin treatment increases survival in small cell lung cancers. Petites Cellules Group. Cancer 74:38-45, 1994.
Lee A J, Kim S G. Selective purging of human multiple myeloma cells from peripheral blood mononuclear cells: a preliminary study. J Blood Med. 2019 Apr. 11; 10:105-109.
Li S, Schmitz K R, Jeffrey P D, Wiltzius J W W, Kussie P, and Ferguson K M. Structural basis for inhibition of the epidermal growth factor receptor by cetuximab. Cancer cells 7:301-311, 2005.
Liberti M V, and Locasale J W. The Warburg effect: how does it benefit cancer cells? Trends Biochem Sci 41(3):211-218, 2016.
Lindahl U, Backstrom G, Thunberg L, and Leder I G. Evidence for a 3-O-sulfated D-glucosamine residue in the antithrombin-binding sequence of heparin. Proc Natl Acad Sci 77:6551-6555, 1980.
Liu A, Ye Y, Chen W, Wang X, and Chen F. Expression of V_{H}-linker-V_{L} orientation-dependent single-chain Fv antibody fragment derived from hybridoma 2E6 against aflatoxin B₁ in Escherichia coli. J Ind Microbiol Biotechnol 42:255-62, 2015.
López-Requena A, Mateo de Acosta C, Pérez A, Valle A, Lombardero J, Sosa K, Pérez R, and Vázquez A M. Chimeric anti-N-glycolyl-ganglioside and its anti-idiotypic MAbs: immunodominance of their variable regions. Hybridoma Hybridomics 22(4): 235-243, 2003.
Luo D, Mah N, Krantz M, Wilde K, Wishart D, Zhang Y, Jacobs F, and Martin L. VI-Linker-Vh orientation-dependent expression of single chain Fv containing an engineered disulfide-stabilized bond in the framework regions. J Biochem 118:825-31, 1995.
Lv H, Yu G, Sun L, Zhang Z, Zhao X, and Chai W. Elevated level of glycosaminoglycans and altered sulfation pattern of chondroitin sulfate are associated with differentiation status and histological type of human primary hepatic carcinoma. Oncology 72:347-356, 2007.
Lyon M, Deakin J A, and Gallagher J T. The mode of action of heparan and dermatan sulfates in the regulation of Hepatocyte Growth Factor/Scatter Factor. J Biol Chem 277(2):1040-1046, 2002. Lyon M, Deakin J A, Rahmoune H, Fernig D G, Nakamura T, and Gallagher J T. Hepatocyte Growth Factor/Scatter Factor binds with high affinity to dermatan sulfate. J Biol Chem 273(1):271-278, 1998.
Ma S-N, Mao Z-X, Wu Y, Liang M-X, Wang D-D, Chen X, Chang P-a, Zhang W, and Tang J-H. The anti-cancer properties of heparin and its derivatives: a review and prospect. Cell Adhesion & Migration 14(1):118-128, 2020.
MacDonald G C, Entwistle J, Lewis K, Maiti P K, Fast D G, Dan M D, and Kaplan H A: Effect of an unconjugated human scFv antibody fragment on growth and metastasis of a breast adenocarcinoma in athymic mice. Ninety-first Annual Meeting of the American Association for Cancer Research, San Francisco, CA, Apr. 1-5, 2000
MacDonald G, Rasamoelisolo M, Entwistle J, Cizeau J, Bosc D, Cuthbert W, Kowalski M, Spearman M, and Glover N. A phase I clinical study of VB4-845: Weekly intratumoral administration of an anti-EpCAM recombinant fusion protein in patients with squamous cell carcinoma of the head and neck. Drug Design, Development and Therapy 2:105-114, 2008.
Maiti P K, Dan M D, Prashar A K, Yuan X Y, Duncan D V, He X, Friesen A D, and Kaplan H A. Development and characterization of pancarcinoma specific human monoclonal antibody H11 (NOVOMAb-G2). Conner T H and Fox F (eds), Biotechnology International 1, Universal Medical Press, Inc., San Francisco, CA, pp 85-93, 1997.
Maiti P, Narang S, Foote S, MacKenzie R, Young M, Xie W, Wang Z, Entwistle J, He X, Reilly R, Dan M, Friesen A, and Kaplan H: The development and characterization of single-chain Fv fragment of human pancarcinoma monoclonal antibody H11. Sixth International Conference on Human Antibodies and Hybridomas, Washington, DC, Apr. 15-17, 1998.
Margalit H, Fischer N, and Ben-Sasson S A. Comparative analysis of structurally defined heparin binding sequences reveals a distinct spatial distribution of basic residues. J Biol Chem 268:19228-19231, 1993.
Marques C, Reis C A, Vivès R A, and Magalhães A. Heparan sulfate biosynthesis and sulfation profiles as modulators of cancer signalling and progression. Front Oncol 11:778752, 2021.
Minsky B B, Dubin P L, and Kaltashov I A. Electrostatic forces as dominant interactions between proteins and polyanions: an ESI-MS study of fibroblast growth factor binding to heparin oligomers. J Am Soc Mass Spectrom 28(4):758-767, 2017.
Narang S, Maiti P, Foote S, Mackenzie R, Young M, Xie W, Wang Z, Entwistle J, He X, de Auraugo A, Xie Y, Fast D, Reilly R, Dan M, Friesen A, and Kaplan H: The development of a human pancarcinomic scFv: from cloning to clinical trials. Tenth International Congress of Immunology, New Delhi, India, Nov. 1-6, 1998
Onyeisi J O S, Ferreira B Z F, Nader H B, and Lopes C C. Heparan sulfate proteoglycans as targets for cancer therapy: a review. Cancer Biol Therapy 21(12) 1087-1094, 2020.
Orecchia P, Conte R, Balza E, Petretto A, Mauri P, Mingari M, and Carnemolla B. A novel human anti-syndecan-1 antibody inhibits vascular maturation and tumour growth in melanoma. Eur J Cancer 49(8):2022-2033, 2013.
Orecchia P, Conte R, Balza E, Pietra G, Mingari M, and Carnemolla B. Targeting Syndecan-1, a molecule implicated in the process of vasculogenic mimicry,
enhances the therapeutic efficacy of the L19-IL2 immunocytokine in human melanoma xenografts. Oncotarget 6:37426-37442, 2015.
Patel D, Bassi R, Hooper A, Prewett M, Hicklin D J, and Kang X. Anti-epidermal growth factor receptor monoclonal antibody cetuximab inhibits EGFR/HER-2 heterodimerization and activation. Int J Oncol 34:25-32, 2009.
Percheson P B, Connors J M, Dan M D, Reilly R M, Kaplan H A, Maiti P K, and St.-Onge J-M: A phase I clinical study of ¹¹¹indium-labeled NovoMAb-G2-scFv, a recombinant human monoclonal antibody fragment, in patients with non-Hodgkin's lymphoma. AACR/NCI/EORTC International Conference: “Molecular Targets & Cancer Therapeutics—Discovery, Development, and Clinical Validation”, Washington, DC, Nov. 16-19, 1999
Perez A, Lombardero J, Mateo C, Mustelier G, Alfonso M, Vazquez A M, and Perez R. Immunogenetic analysis of variable regions encoding AB1 and γ-type AB2 antibodies from the NeuGc-containing ganglioside family. Hybridoma 20(4):211-221, 2001.
Pfankuchen D B, Stolting D B, Schlesinger M, Royer H-D, and Bendas G. Low molecular weight heparin tinzaparin antagonizes cisplatin resistance in ovarian cancer cells. Biochem Pharmacol 97:147-157, 2015.
Phillips P G, Yalcin M, Cui H, Abdel-Nabi H, Sajjad M, Bernacki R, Veith J, and Mousa S A. Increased tumor uptake of chemotherapeutics and improved chemoresponse by novel non-anticoagulant low molecular weight heparin. Anticancer Res 31:411-419, 2011. Press Release, Jun. 17, 1998, “Novopharm Biotech completes NovoMAb-G2 IgM clinical study”.
Press Release, Mar. 25, 1999, “Novopharm Biotech announces results of Phase I clinical study”.
Press Release, May 26, 1999, “Novopharm Biotech Inc. announces evidence of anti-cancer activity utilizing fully human antibodies in pre-clinical animal studies”.
Press Release, Oct. 7, 1999, “Anti-cancer treatment shows potential to significantly reduce spread of breast cancer”.
Press Release, Apr. 4, 2000, “Novopharm Biotech's anti-cancer drug H11 (NovoMAb-G2 scFv) reduces the formation of secondary tumors in human breast tumor model”.
Press Release, Jul. 23, 2002, “Viventia announces positive results of VB2-011 Phase I/II clinical trial”.
Price M A, Colvin Wanshura L E, Yang J, Carlson J, Xiang B, Li G, Ferrone S, Dudek A Z, Turley E A, and McCarthy J B. CSPG4, a potential therapeutic target, facilitates malignant progression of melanoma. Pigment Cell Melanoma Res 24:1148-1157, 2011.
Pudełko A, Wisowski G, Olczyk K, and Kozma M. The dual role of the glycosaminoglycan chondroitin-6-sulfate in the development, progression and metastasis of cancer. FEBS J 286: 1815-1837, 2019.
Ramjiawan B, Ariano R E, Mantsch H H, Maiti P, and Jackson M. Immunifluorescence imaging as a tool for studying the pharmacokinetics of a human monoclonal single chain fragment antibody. IEEE Trans Med Imaging 21(10):1317-1323, 2002.
Ramjiawan B, Maiti P, Aftanas A, Kaplan H, Fast D, Mantsch H H, and Jackson M. Noninvasive localization of tumors by immunofluorescence imaging using a single chain Fv fragment of a human monoclonal antibody with broad cancer specificity. Cancer 89(5):1134-1144, 2000.
Ramjiawan B, Maiti P, Kaplan H, Fast D, Mantsch H H, and Jackson M. In vivo monitoring of the distribution of a monoclonal antibody fragment. Vibrational Spectroscopy 28:177-188, 2002.
Reilly R M, Maiti P K, Kiarash R, Prashar A K, Fast D G, Entwistle J, Dan M D, Narang S A, Foote S, and Kaplan H A. Rapid imaging of human melanoma xenografts using an scFv fragment of the human monoclonal antibody H11 labelled with ¹¹¹In. Nucl Med Comm 22:587-595, 2001.
Sarkar A, and Desai U R. A simple method for discovering druggable, specific glycosaminoglycan-protein systems. Elucidation of key principles from heparin/heparan sulfate-binding proteins. PLoS ONE 10(10):e0141127, 2015.
Sarrazin S, Lamanna W C, and Esko J D. Heparan sulfate proteoglycans. Cold Spring Harb Perspect Biol 3:a004952, 2011.
Schoonbroot S, Steukers M, Viswanathan M, Frans N, Timmermans M, Wehnert A, Nguyen M, Ladner R C, and Hoet R M. Engineering antibody heavy chain CDR3 to create a phage display Fab library rich in antibodies that bind charged carbohydrates. J Immunol 181:6213-6221, 2008.
Shi D, Sheng A, and Chi L. Glycosaminoglycan-protein interactions and their roles in human disease. Front Mol Biosci 8:639666, 2021.
Slamon D J, Godolphon W, Jones L A, Holt J A, Wong S G, Keith D E, Levin W J, Stuart S G, Udove J, and Ullrich A, et al. Studies of the HER-2/Neu proto-oncogene in human breast and ovarian cancer. Science 244:707-712, 1989.
Soriano J L, Batista N, Santiesteban E, Lima M, Gonzalez J, Garcia R, Zarza Y, López M V, Rodrigez M, Loys J L, Montejo N, Aguirre F, Macías A, and Vázquez A M. Metronomic cyclophosphamide and methotrexate chemotherapy combined with 1E10 anti-idiotype vaccine in metastatic breast cancer. Int J Breast Cancer 2011:710292, 2011.
Soto Y, Acosta E, Delgado L, Pérez A, Falcón V, Bécquer M A, Fraga A, Brito V, Álvarez I, Griñan T, Fernández-Marrero Y, López-Requena A, Noa M, Fernández E, and Vázquez A M. Antiatherosclerotic effect of an antibody that binds to extracellular matrix glycosaminoglycans. Arterioscler Thromb Vasc Biol 32:595-604, 2012.
Soto Y, Mesa N, Alfonso Y, Pérez A, Batlle F, Griñán T, Pino A, Viera J, Frómeta M, Brito V, Olivera A, Zayas F, and Vázquez A. Targeting arterial wall sulfated glycosaminoglycans in rabbit atherosclerosis with a mouse/human chimeric antibody. mAbs 6(5):1340-1346, 2014.
Straus A H, Travassos L R, and Takahashi H K. A monoclonal antibody (ST-1) directed to the native heparin chain. Analytical Biochem 201:1-8, 1992.
Stuchliková E, Ježková Z, Soukupová K, and Vich Z. Complement fixing anti-heparin antibodies in human serum in relationship to age, arteriosclerosis, lipid disorders and neoplastic diseases. Geront Clin 12:186-192, 1970.
Suarez, E R, Paredes-Gamero E J, Del Giglio A, dos Santos Tersariol I L, Nader H B, and Silva Pinhal M A. Heparan sulfate mediates trastuzumab effect in breast cancer cells. BMC Cancer 13:444, 2013.
Tajima H, Matsumoto K, and Nakamura T. Regulation of cell growth and motility by hepatocyte growth factor and receptor expression in various cell species. Exp Cell Res 202:423-431, 1992.
Tilmont, R., Yakoub-Agha, I., Eikema, D J. et al. Carfilzomib, lenalidomide and dexamethasone followed by a second ASCT is an effective strategy in first relapse multiple myeloma: a study on behalf of the Chronic malignancies working party of the EBMT. Bone Marrow Transplant 58, 1182-1188 (2023).
Trowbridge J M and Gallo R L. Dermatan sulfate: new functions from an old glycosaminoglycan. Glycobiology 12(9):117R-125R, 2002.
Ucakturk E, Akman O, Sun X, Baydar D E, Dolgun A, Zhang F, and Linhardt R J. Changes in composition and sulfation patterns of glycoaminoglycans in renal cell carcinoma. Glycoconj J 33:103-112, 2016.
Vallen M J E, Schmidt S, Oosterhof A, Bulten J, Massuger L F A G, and van Kuppevelt T H. Primary ovarian carcinomas and abdominal metastasis contain 4,6-disulfated chondroitin sulfate rich regions, which provide adhesive properties to tumour cells. PLoS ONE 9(11):e111806, 2014.
Vallet S D, Clerc O, and Ricard-Blum S. Glycosaminoglycan-protein interactions: the first draft of the glycosaminoglycan interactome. J Histochem Cytochem 69(2):93-104, 2020.
van de Westerlo EMA, Smetsers T F C M, Dennissen MABA, Linhardt R J, Veerkamp J H, van Muijen G N P, and van Kuppevelt T H. Human single chain antibodies against heparin: selection, characterization, and effect on coagulation. Blood 99:2427-2433, 2002.
van den Born J, Salmivirta K, Henttinen T, Ostman N, Ishimaru T, Miyaura S, Yoshida K, and Salmivirta M. Novel heparan sulfate structures revealed by monoclonal antibodies. J Biol Chem 280(21): 20516-20523, 2005.
van Kuppevelt T H, Dennissen MABA, van Venrooij W J, H RMA, and Veerkamp J H. Generation and application of type-specific anti-heparan sulfate antibodies using phage display technology: further evidence for heparan sulfate heterogeneity in the kidney. J Biol Chem 273(21):12960-12966, 1998.
Vázquez A M, Alfonso M, Lanne B, Karlsson K-A, Carr A, Barroso O, Fernández L E, Rengifo E, Lanio M E, Alvarez C, Zeuthen J, and Pérez R. Generation of a murine monoclonal antibody specific for N-glycolylneuraminic acid-containing gangliosides that also recognizes sulfated glycolipids. Hybridoma 14(6):551-556, 1995.
Vázquez A M, Pérez A, Hernández A M, Macías A, Alfonso M, Bombino G, and Pérez R. Syngeneic anti-idiotypic monoclonal antibodies to an anti-NeuGc-containing ganglioside monoclonal antibody. Hybridoma 17(6):527-534, 1998.
Weissmann M, Bhattacharya U, Feld S, Hammond E, Ilan N, and Vlodavsky I. The heparanase inhibitor PG545 is a potent anti-lymphoma drug: mode of action. Matrix Biol 77:58-72, 2019.
Wijdenes J, Vooijs W C, Clement C, Post J, Morard F, Vita N, Laurent P, Sun R X, Klein B, and Dore J M. A plasmocyte selective monoclonal antibody (B-B4) recognizes syndecan-1. Br J Haematol 94:318-323, 1996.
Xu D, and Esko J D. Demystifying heparan sulfate-protein interactions. Annu Rev Biochem 83:129-57, 2014.
Yamaguchi K, Tamaki H, and Fukui S. Detection of oligosaccharide ligands for Hepatocyte growth factor/Scatter factor (HGF/SF), Keratinocyte growth factor (KGF/FGF-7), RANTES and Heparin cofactor II by neoglycolipid microarrays of glycosaminoglycan-derived oligosaccharide fragments. Glycoconj J 23:513-23, 2006.
Yang H and Wang L. Heparan sulfate proteoglycans in cancer: Pathogenesis and therapeutic potential. Advances in Cancer Research, Volume 157:251-291, 2023.
Yang J, Liao Q, Price M, Moriarity B, Wolf N, Felices M, Miller J S, Geller M A, Bendzick L, Hopps R, Starr T K, O'Connor C H, Tarullo S, Nelson A C, Turley E, Wang J, and McCarthy J B. Chondroitin sulfate proteoglycan 4, a targetable oncoantigen that promotes ovarian cancer growth, invasion, cisplatin resistance and spheroid formation. Translational Oncology 16:101318, 2022.
Zahavi D and Weiner L. Monoclonal antibodies in cancer therapy. Antibodies 9:34, 2020.

1. An antibody that binds to isolated sulfated glycosaminoglycans (GAGs), wherein the antibody comprises a Variable Heavy Chain Complementarity Determining Region 3 (VHCDR3) according to the Kabat numbering system having the amino acid sequence of SEQ ID NO: 6.

2. The antibody of claim 1, wherein the antibody is a single-chain variable fragment (scFv).

3. The antibody of claim 1, wherein the antibody is a human scFv antibody.

4. The antibody of claim 1, wherein said antibody binds to any combination of 2 or more of the following 4 classes of glycosaminoglycan (GAG): heparin/heparan sulfate, chondroitin sulfate/dermatan sulfate, keratan sulfate, and hyaluronic acid.

5. The antibody of claim 1, wherein the antibody comprises:
a VHCDR3 according to the Kabat numbering system having the amino acid sequence of any one of SEQ ID NO: 1-5;
a VHCDR3 according to the Kabat numbering system having the amino acid sequence of SEQ ID NO: 1, 2, 4 or 5; or
a VHCDR3 according to the Kabat numbering system having the amino acid sequence of SEQ ID No: 3.

6. (canceled)

7. The antibody of claim 5, wherein the K_{d} of the antibody and heparin having a degree of polymerization of 24 (dp24) is less than 250 nM, or
wherein the K_{d} of the antibody and chondroitin sulfate D having a degree of polymerization of 20 (dp20) is less than 330 nM.

8. (canceled)

9. An antibody or fragment thereof with a V_{H} or a V_{L} region having at least 70%, at least 80%, or at least 90% homology to the V_{H} or V_{L} amino acid sequence of any one of SEQ ID NO: 7-11 and 21-23, wherein the antibody binds to any combination of 2 or more of the following 4 classes of glycosaminoglycan (GAG): heparin/heparan sulfate, chondroitin sulfate/dermatan sulfate, keratan sulfate, and hyaluronic acid.

10. The antibody of claim 9, wherein
the GAGs comprise isolated heparin, high sulfation heparan sulfate, low/intermediate sulfation heparan sulfate, and chondroitin-2,6-sulfate with 20 degrees of polymerization (dp20); or
wherein the antibody binds to isolated heparin, high sulfation heparan sulfate, low/intermediate sulfation heparan sulfate, and chondroitin-2,6-sulfate with 20 degrees of polymerization (dp20).

11. (canceled)

12. (canceled)

13. The antibody of claim 9, wherein the K_{d} of the antibody and a heparan sulfate octamer with 2.5 sulfates/disaccharide (HS024) under mildly acidic conditions is less than 1 μM.

14. The antibody of claim 13, wherein the mildly acidic conditions correspond to a pH of between 6.0 and 6.8.

15. The antibody of claim 1, comprising:
a methionine in the penultimate position of the VHCDR1 according to the Kabat numbering system;
the amino acid sequence VKG at the C-terminal end of the VHCDR2 according to the Kabat numbering system;
the amino acid sequence YLA at the C-terminal end of the VLCDR1 according to the Kabat numbering system;
a VHCDR1 according to the Kabat numbering system of SEQ ID NO: 12:
a VHCDR2 according to the Kabat numbering system of SEQ ID NO: 13;
a VHCDR1 according to the Kabat numbering system of SEQ ID NO: 14;
a VHCDR2 according to the Kabat numbering system of SEQ ID NO: 15;
a VHCDR3 according to the Kabat numbering system of SEQ ID NO: 16; or a combination thereof.

16-22. (canceled)

23. An antibody:
a.) that binds to heparin, chondroitin-2,6-sulfate with 20 degrees of polymerization (dp20), and dermatan sulfate, wherein the antibody comprises the amino acid sequence of SEQ ID NO: 17, SEQ ID NO: 18, and/or SEQ ID NO: 22;
b.) that binds to heparin, chondroitin-2,6-sulfate with 20 degrees of polymerization (dp20), and dermatan sulfate, wherein the antibody comprises an amino acid sequence of SEQ ID NO: 19, SEQ ID NO: 20, and/or SEQ ID NO: 23;
c.) that is an anti-GAG antibody, comprising a methionine in the penultimate position of the VHCDR1 according to the Kabat numbering system and the binds to any combination of 2 or more of the following 4 classes of glycosaminoglycan (GAG): heparin/heparan sulfate, chondroitin sulfate/dermatan sulfate, keratan sulfate, and hyaluronic acid;
d.) that is an anti-GAG antibody, comprising the amino acid sequence VKG at the C-terminal end of the VHCDR2 according to the Kabat numbering system and that bind to any combination of 2 or more of the following 4 classes of glycosaminoglycan (CAG): heparin/heparan sulfate, chondroitin sulfate/dermatan sulfate, keratan sulfate, and hyaluronic acid;
e.) that is an anti-GAG antibody, comprising the amino acid sequence YLA at the C-terminal end of the VLCDR1 according to the Kabat numbering system and that binds to any combination of 2 or more of the following 4 classes of glycosaminoglycan (GAG): heparin/heparan sulfate, chondroitin sulfate/dermatan sulfate, keratan sulfate, an hyaluronic acid; or
f.) An anti-GAG antibody, that binds to any combination of 2 or more of the following 4 classes of glycosaminoglycan (GAG): heparin/heparan sulfate, chondroitin sulfate/dermatan sulfate, keratan sulfate, and hyaluronic acid and that does not also bind to Neu5Gc-containing gangliosides.

24-33. (canceled)

34. A pharmaceutical composition comprising the antibody of claim 1 and a pharmaceutically acceptable excipient or diluent.

35. (canceled)

36. A method of killing cancer cells, performing an autologous stem cell transplantation (ASCT) of autologous bone marrow transplantation (ABMT) or treating cancer in a subject in need thereof, wherein:
killing cancer cells and/or performing an autologous stem cell transplantation (ASCT) or autologous bone marrow transplantation (ABMT) comprises exposing the cancer cells or the hematopoietic stem cells (HSC) of a subject to an effective amount of an anti-GAG antibody, wherein said antibody binds to any combination of 2 or more of the following 4 classes of glycosaminoglycan (GAG): heparin/heparan sulfate, chondroitin sulfate/dermatan sulfate, keratan sulfate, and hyaluronic acid, and wherein the anti-GAG antibody does not bind to Neu5Gc-containing gangliosides, the anti-GAG antibody is polyspecific, and/or the anti-GAG antibody is the antibody as defined in claim 1; or
wherein treating cancer in a subject in need thereof comprises
administering a therapeutically effective amount of the antibody of claim 1 to the subject.

37. The method of claim 36, wherein the cancer aberrantly expresses a membrane-tethered heparan sulfate proteoglycan.

38. The method of claim 37, wherein the membrane-tethered heparan sulfate proteoglycan is syndecan-1, syndecan-2, syndecan-3, syndecan-4, glypican-1, glypican-2, glypican-3, glypican-4, glypican-5, glypican-6, glypican-7, glypican-8, or any combination thereof.

39. The method of claim 36,
wherein treating cancer in a subject in need thereof further comprises:
administering a chemotherapeutic agent to the subject;
another anti-cancer agent to the subject; or
a combination thereof;
wherein the method for killing cancer cells is for directly killing cancer cells; or
wherein the hematopoietic stem cells (HSC) are contaminated with, or presumed to be contaminated with, cancer cells;
exposing the autologous hematopoietic stem cells (HSCs) to the anti-GAG antibody kills cancer cells, thereby purging the cancer cells from the autologous HSCs of the subject; and/or
the subject has been diagnosed with cancer.

40-56. (canceled)

57. The method of claim 39, wherein the cancer is multiple myeloma, Hodgkin's lymphoma or neuroblastoma.

58-61. (canceled)

62. The antibody of claim 7, comprising:
a methionine in the penultimate position of the VHCDR1 according to the Kabat numbering system;
the amino acid sequence VKG at the C-terminal end of the VHCDR2 according to the Kabat numbering system;
the amino acid sequence YLA at the C-terminal end of the VLCDR1 according to the Kabat numbering system;
a VHCDR1 according to the Kabat numbering system of SEQ ID NO: 12;
a VHCDR2 according to the Kabat numbering system of SEQ ID NO: 13;
a VLCDR1 according to the Kabat numbering system of SEQ ID NO: 14;
a VLCDR2 according to the Kabat numbering system of SEQ ID NO: 15;
a VLCDR3 according to the Kabat numbering system of SEQ ID NO: 16; or
a combination thereof.

63. A pharmaceutical composition comprising the antibody of claim 9 and a pharmaceutically acceptable excipient or diluent.
